SEQUENCE LISTING (81 sequences)

SEQ ID NO: 1 - DNA, 18 bp, Pisum sativum
  source 1..18: mol_type = genomic DNA
attcttcagatccgtttg

SEQ ID NO: 2 - DNA, 27 bp, Pisum sativum
  source 1..27: mol_type = genomic DNA
attaaattttcagatctgattatgtat

SEQ ID NO: 3 - DNA, 55 bp, Pisum sativum
  source 1..55: mol_type = genomic DNA
acgtaattcttcagatccgtttgttatgattaaattttcagatctgattatgtat

SEQ ID NO: 4 - DNA, 683 bp, Pisum sativum
  source 1..683: mol_type = genomic DNA
gtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 5 - DNA, 739 bp, Pisum sativum
  source 1..739: mol_type = genomic DNA
gtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacacgtaattcttcagatccgtttgttatgattaaattttcagatctgattatgtatcaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 6 - DNA, 2548 bp, Pisum sativum
  source 1..2548: mol_type = genomic DNA
gttgctgaatcagaagttgagtgaggtttggaggcaactgttgggacaacggcttagacaaacattttgatgctacaatttatccaatttttagagggttttcatttacgccctatatatttttcagaaattttatggaagttcaaaagttttcttgttgatgcgtctattacgttctttcatttgatctaatatgagtgtgttcgaaaattatctttaggagattattatgaaataaaatagaaagagtttgtttgaaagtaaactttaggacactgaaaatagaatctctgaaaaatatataggataaaatgtaacgagtaattctacatttttataaattttggaaccactcaattttgtcaacgagtttcagtcttctgctagagtttcggaaattattaccgctttcttcttgtcactgttctgtatttatataacatcatctgtgagagtttcgaaaattgttacagctttcttctgaccactgttctgtatttctataacagattaaatgaatctaaaaatcaaaatctacttataaaagagaatagagggtatatacacgtttattatttaatgaacccaaatgaattgtcttctatatatattagtttatttttagtcatatccaactttatgttgcaatcttaatagtggactaaaaaagatctaaaaatattaattcacaagctaatcaaagcatgactactatacctaatgttgttttaagaaaaaaatttcacaacataggtaaacatataactaaatttttatattgagttttactgtatacttataaaatatttgacaattttttttcaaagatacattgattagctgttctcaaatatccaatttcaaatacataagacaatacttttcaaacacaaattttatttgatgtcgggcttatcattttgagaaattgtctttttatagaagttgagcacacttaattttattgttcatttctttattataacttcacgatgtgtatgcaacattagggcagtttgttttaactttaagaaaataattttcttttaaatggattttacaaaaatgttttttaaaatattataagtttttctaaatttatttttgataaattaaaaatttgaattattcattctataatataaatatacattattaaagatcaaaacatagtcaaaatcacaattttttcaaaaaaattatatcttaaaaataatttttatgaaaagctatttgaaatagcttcaaaattaagtgattttttaaaattttgatatctaaattttttttcgataaaatgatgaaatacctaaaataacattttaagaataattattcaaaccaaatttttatttgaagtttttatgaaaagtttttttataaactttatttagattaaaatatgtaacactgtaacaatcatttaaaaaaaaaccaaaacaaatggatcctaaattaattaaatttttttttgagatttttctgatacattccaatttatattatcggtgctcatcccaaattcactatatcatttgtattcttttaaaaattaattttggcattttttagttatacgcactttccacgaaataagccaacagattcaagacgtgtttggatggcaaagctatcatcttatccgttgccgaagacgactgtgataccaaacattgccgtgtcaaaatcgtttatagctcagatttgtccacgtgtcgcgatgtgattggaagagctgccgttacttttacgcaacctcgaatcattttaagctataaattcgaaagcccaatttcacagctttcatatattcatatactttcgtttcgttgcagagaagagaaaccctagcacactgaatcaaggtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 7 - DNA, 1990 bp, Pisum sativum
  source 1..1990: mol_type = genomic DNA
cttcgttcctaccatgcgcaagaagttagaaccattttcgctcgaatgtgacaagaacaaatatgtgagaatctagattatgttatccattatgatgtgatgccaatgttcacttttccctgttttaggtagcaagtaatgttttcagttcgaatgaaactaagtacctttcactaaaatcgacctacaaacaaacatttcctacccataactatttaaccttgagttctccatcgcaccgggagatacgtatgagcaagatccgcaatcttgtcaggcacattaataaataaaaaattgtgaccctttttttgtctttctattttacacttttaattactcaaagaaaacaaataatgtaagctaacatttaatcacaagtttaataaaaaaggttcttgttgagtacaacaaacgtgaggggtgctaataccttccctttgcataatcgatctcgaacccgaatttgattgcgatgaccatcttctttttcttttaagagttttatcaatattttccctttccttccataataaataaagttcgttggcgactatgtcatctcgagcgtgcaggtgcttgtggtcatttttcgctctatgacacctttaagtaaaccactttctcttttttatggaacttattcgttctggctttcttagctttcaggagttcgacctgtcgaactctgtcagccaaagagaaaaggcctaataatgaacaacatatcattggacgtaaaacaaatataatatttcttctcactgcatgcgatccttcacacaaatgtcttcctaaccatccaggttaacctaacgtccaaccgcatcccacgccatgccacacattttcagttgtcaatcatttttccgatttaaagaaagaagcatacaatttctcaagtattcaaaccatttctcattcaaacaccactttgcactctcatactgattcgagcattaaagtgttcacattataagttcgtcccctcttcaccgtatcaaaagctcagagccaccgcaacaaactctacatcctcactctccgatcaactctagtatcactacggaaaaaaatattcatcgtatctctcatttaaatttctatggcattatcctttgttacttcttttaatagtatttttcttatcttaaaaagaattattttttttacaaaaagtgtgaaccactattttattttgttctccttataaagaaattatatatttttctaatctatataatttcacaacagccggccttccaacggtcgagtgatccatatctaaaacgtgggcccaaataaaaatcaaagacccaataaactaacgccgtgataacgtttaagcaaaattagaccacatacatttaacttcgtcaatttgtaacaccaactagcaccttccaaattgcaatttctttagttctttccgccccgaacgcatcggatactaaatttggccgcccataaaaaaccagccaacaacttgcacctaatgacacgtcatcaagccacgtgtcaaaaacatagtggtgaagatttcgacccctcttgctataaaacgacccttctcaccttcatcatttcacagttcaatttcaaacgaacaaattcaatccatttcttcgtgacctaacttctcctcaaggtattttctctatttgattttctctattgattttcttttcgcttctaaatatcgtagatctttcaaaaatcgatcttgtaggatttgtaattgcttaattgttcatgagtttctctctgatctgatcctaatttgatgaattaacaatcaattgttcgtttctcttggatctctttagatccgattgttaaaagctttttttttgttaaggtgtagtgaattcaattcaaattttagctttgtatgaacgaaattaatagcgttattacgtaattcttcagatccgtttgttatgatgttttaattctgattaatttgtatctgttgattccgattttccag

SEQ ID NO: 8 - DNA, 2104 bp, Pisum sativum
  source 1..2104: mol_type = genomic DNA
gtggacatgtgagccaatgagagacacctagctaaactaaatagctagtgaatggtcaaaattctccctcatagtcatattacaaatatttaacggcagtgttaaattgagtttgaaaaaacttgtgaaataatttaaaggcatgaaatgtctcaaacttatgtttaaggaaatatatcaaagtatacttggaataagcattaataaatgcaacataataaataaagcttgaggtagaagaaaagggtgcaaaaccccaaagattcgtgtacactagttcaaaaggagaatgatacatggttatagatgaatgagcatgtagtctatgcgattttccaatacaacaagacttgcacgataatgtttgaattatgagaaactgaaatattataatgctttagagcttgtctaatagctgaataatttgtatgacctagcctaaaatgccaaagaaggctagaactaggtttatttacactagatatcatagaacatgaaataaactttgcattagtgcaagtagaagtagaactagagcaagacacaaagggcattcttgaaggctccacaaccaagccaaaaaagtaatacaatccacttgagttacagaaaccttcaagaaaagtgagtctagaaacttgagacttaatgaagcatttgtcagtcaagaattcaaacacaacatcattatcacgcggaaacttagatactgaccacaaattccttataatacaaggtaccatgagaatatcactaagaacaagtgttgttaagagacataaagagaaagaaatacgagttgaacctacgcacctaattgagaaagattgaccattaggaactctaacacaaaatataatgcaagtaagaagcatgtgaagtaaggtggtgagaggagaaacaccaatgatatgtattactcagattattcattcaaccttcatatgcatgtttgtattcatgatatattcacatacatgaatgtatatactaagctatctatcttactacctaagctatcctcctaataatgcgaaaccaaacaaagctaaggatggctttttctatagtgaagcttgcctccgtttctttcttttcttttttaaaagaaagcgttggcgtattgcaagggttgagagcgtaccgtttgtccaggtgtcacattttgatacgcataatttgccgttgccgtaacgaaaccttctatcctatatcgttttttggatataaaaccaaatcccgtaaacccaattttgacaaagaaaagagattttcatttctacacacagcacacaacaccaccgtttcctttcgttgcaaagagaagagattttcatttctacacacagcacacaacaccaccgtttcaaggtatcatcgattttcatctcctcccaattatgacctagattataataactatctttcaatcgatctttagatctctaatatctgaaatttatacgtgtttctatacatcgattattagggtttgtgttattttttaatctatatttcgatctatcggttttgtttttcatctgattttctccgcgtcattgaaacgtttacatgatctaaatctctcaataatcactcaatcggaggtatatgtgaaactttcttgattcgttcaattttgtgttgatcaaggtcgatcttttgatttatgataacgctgttaattaaattttcggatctgattatgtattctatgattctagaagtcctttaattggattagtttaggatattgattcgattgatttattgattggaaatgatttattcaagtctaattttgtaatcaggttgatctgtgtacacgcgctgtattaaattttcagatctgattatgtattctatgaatctagagttctctaattggattagtttagggatattgatttcgatttatatattgatttgaaatgatttattgaattgtaattttgtaatcagggttgttgtttgatttaatgattatttaggagttttgttgattgcatatttgaaatgttgtgtatatagaattgattatttctactgatgttatacttgtagtgattgattcattgttttcgtttgtttttgtatgatatag

SEQ ID NO: 9 - DNA, 2576 bp, synthetic construct
  source 1..2576: mol_type = other DNA
gttgctgaatcagaagttgagtgaggtttggaggcaactgttgggacaacggcttagacaaacattttgatgctacaatttatccaatttttagagggttttcatttacgccctatatatttttcagaaattttatggaagttcaaaagttttcttgttgatgcgtctattacgttctttcatttgatctaatatgagtgtgttcgaaaattatctttaggagattattatgaaataaaatagaaagagtttgtttgaaagtaaactttaggacactgaaaatagaatctctgaaaaatatataggataaaatgtaacgagtaattctacatttttataaattttggaaccactcaattttgtcaacgagtttcagtcttctgctagagtttcggaaattattaccgctttcttcttgtcactgttctgtatttatataacatcatctgtgagagtttcgaaaattgttacagctttcttctgaccactgttctgtatttctataacagattaaatgaatctaaaaatcaaaatctacttataaaagagaatagagggtatatacacgtttattatttaatgaacccaaatgaattgtcttctatatatattagtttatttttagtcatatccaactttatgttgcaatcttaatagtggactaaaaaagatctaaaaatattaattcacaagctaatcaaagcatgactactatacctaatgttgttttaagaaaaaaatttcacaacataggtaaacatataactaaatttttatattgagttttactgtatacttataaaatatttgacaattttttttcaaagatacattgattagctgttctcaaatatccaatttcaaatacataagacaatacttttcaaacacaaattttatttgatgtcgggcttatcattttgagaaattgtctttttatagaagttgagcacacttaattttattgttcatttctttattataacttcacgatgtgtatgcaacattagggcagtttgttttaactttaagaaaataattttcttttaaatggattttacaaaaatgttttttaaaatattataagtttttctaaatttatttttgataaattaaaaatttgaattattcattctataatataaatatacattattaaagatcaaaacatagtcaaaatcacaattttttcaaaaaaattatatcttaaaaataatttttatgaaaagctatttgaaatagcttcaaaattaagtgattttttaaaattttgatatctaaattttttttcgataaaatgatgaaatacctaaaataacattttaagaataattattcaaaccaaatttttatttgaagtttttatgaaaagtttttttataaactttatttagattaaaatatgtaacactgtaacaatcatttaaaaaaaaaccaaaacaaatcgatcctaaattaattaaatttttttttgagatttttctgatacattccaatttatattatcggtgctcatcccaaattcactatatcatttgtattcttttaaaaattaattttggcattttttagttatacgcactttccacgaaataagccaacagattcaagacgtgtttggatggcaaagctatcatcttatccgttgccgaagacgactgtgataccaaacattgccgtgtcaaaatcgtttatagctcagatttgtccacgtgtcgcgatgtgattggaagagctgccgttacttttacgcaacctcgaatcattttaagctataaattcgaaagcccaatttcacagctttcatatattcatatactttcgtttcgttgcagagaagagaaaccctagcacactgaatcaaggtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacacgtaattcttcagatccgtttgttatgaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 10 - DNA, 2576 bp, synthetic construct
  source 1..2576: mol_type = other DNA
gttgctgaatcagaagttgagtgaggtttggaggcaactgttgggacaacggcttagacaaacattttgatgctacaatttatccaatttttagagggttttcatttacgccctatatatttttcagaaattttatggaagttcaaaagttttcttgttgatgcgtctattacgttctttcatttgatctaatatgagtgtgttcgaaaattatctttaggagattattatgaaataaaatagaaagagtttgtttgaaagtaaactttaggacactgaaaatagaatctctgaaaaatatataggataaaatgtaacgagtaattctacatttttataaattttggaaccactcaattttgtcaacgagtttcagtcttctgctagagtttcggaaattattaccgctttcttcttgtcactgttctgtatttatataacatcatctgtgagagtttcgaaaattgttacagctttcttctgaccactgttctgtatttctataacagattaaatgaatctaaaaatcaaaatctacttataaaagagaatagagggtatatacacgtttattatttaatgaacccaaatgaattgtcttctatatatattagtttatttttagtcatatccaactttatgttgcaatcttaatagtggactaaaaaagatctaaaaatattaattcacaagctaatcaaagcatgactactatacctaatgttgttttaagaaaaaaatttcacaacataggtaaacatataactaaatttttatattgagttttactgtatacttataaaatatttgacaattttttttcaaagatacattgattagctgttctcaaatatccaatttcaaatacataagacaatacttttcaaacacaaattttatttgatgtcgggcttatcattttgagaaattgtctttttatagaagttgagcacacttaattttattgttcatttctttattataacttcacgatgtgtatgcaacattagggcagtttgttttaactttaagaaaataattttcttttaaatggattttacaaaaatgttttttaaaatattataagtttttctaaatttatttttgataaattaaaaatttgaattattcattctataatataaatatacattattaaagatcaaaacatagtcaaaatcacaattttttcaaaaaaattatatcttaaaaataatttttatgaaaagctatttgaaatagcttcaaaattaagtgattttttaaaattttgatatctaaattttttttcgataaaatgatgaaatacctaaaataacattttaagaataattattcaaaccaaatttttatttgaagtttttatgaaaagtttttttataaactttatttagattaaaatatgtaacactgtaacaatcatttaaaaaaaaaccaaaacaaatcgatcctaaattaattaaatttttttttgagatttttctgatacattccaatttatattatcggtgctcatcccaaattcactatatcatttgtattcttttaaaaattaattttggcattttttagttatacgcactttccacgaaataagccaacagattcaagacgtgtttggatggcaaagctatcatcttatccgttgccgaagacgactgtgataccaaacattgccgtgtcaaaatcgtttatagctcagatttgtccacgtgtcgcgatgtgattggaagagctgccgttacttttacgcaacctcgaatcattttaagctataaattcgaaagcccaatttcacagctttcatatattcatatactttcgtttcgttgcagagaagagaaaccctagcacactgaatcaaggtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacattaaattttcagatctgattatgtatcaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 11 - DNA, 2604 bp, synthetic construct
  source 1..2604: mol_type = other DNA
gttgctgaatcagaagttgagtgaggtttggaggcaactgttgggacaacggcttagacaaacattttgatgctacaatttatccaatttttagagggttttcatttacgccctatatatttttcagaaattttatggaagttcaaaagttttcttgttgatgcgtctattacgttctttcatttgatctaatatgagtgtgttcgaaaattatctttaggagattattatgaaataaaatagaaagagtttgtttgaaagtaaactttaggacactgaaaatagaatctctgaaaaatatataggataaaatgtaacgagtaattctacatttttataaattttggaaccactcaattttgtcaacgagtttcagtcttctgctagagtttcggaaattattaccgctttcttcttgtcactgttctgtatttatataacatcatctgtgagagtttcgaaaattgttacagctttcttctgaccactgttctgtatttctataacagattaaatgaatctaaaaatcaaaatctacttataaaagagaatagagggtatatacacgtttattatttaatgaacccaaatgaattgtcttctatatatattagtttatttttagtcatatccaactttatgttgcaatcttaatagtggactaaaaaagatctaaaaatattaattcacaagctaatcaaagcatgactactatacctaatgttgttttaagaaaaaaatttcacaacataggtaaacatataactaaatttttatattgagttttactgtatacttataaaatatttgacaattttttttcaaagatacattgattagctgttctcaaatatccaatttcaaatacataagacaatacttttcaaacacaaattttatttgatgtcgggcttatcattttgagaaattgtctttttatagaagttgagcacacttaattttattgttcatttctttattataacttcacgatgtgtatgcaacattagggcagtttgttttaactttaagaaaataattttcttttaaatggattttacaaaaatgttttttaaaatattataagtttttctaaatttatttttgataaattaaaaatttgaattattcattctataatataaatatacattattaaagatcaaaacatagtcaaaatcacaattttttcaaaaaaattatatcttaaaaataatttttatgaaaagctatttgaaatagcttcaaaattaagtgattttttaaaattttgatatctaaattttttttcgataaaatgatgaaatacctaaaataacattttaagaataattattcaaaccaaatttttatttgaagtttttatgaaaagtttttttataaactttatttagattaaaatatgtaacactgtaacaatcatttaaaaaaaaaccaaaacaaatcgatcctaaattaattaaatttttttttgagatttttctgatacattccaatttatattatcggtgctcatcccaaattcactatatcatttgtattcttttaaaaattaattttggcattttttagttatacgcactttccacgaaataagccaacagattcaagacgtgtttggatggcaaagctatcatcttatccgttgccgaagacgactgtgataccaaacattgccgtgtcaaaatcgtttatagctcagatttgtccacgtgtcgcgatgtgattggaagagctgccgttacttttacgcaacctcgaatcattttaagctataaattcgaaagcccaatttcacagctttcatatattcatatactttcgtttcgttgcagagaagagaaaccctagcacactgaatcaaggtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacacgtaattcttcagatccgtttgttatgattaaattttcagatctgattatgtatcaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 12 - DNA, 2345 bp, Arachis hypogaea
  source 1..2345: mol_type = genomic DNA
gtgtatgcagtaatttattggcttacgacagattaattcttaacctgtcgaattggaataggaatatcggagcaatcaaacaaaatagttaccaaataaatacgagtattttattcattttctatctaaagtggatattgattttatcctttgtatctctacgatattgaaaacaatgataagaaacatgttttgttaactagtgtcgtctatacctaatacaacaaacttactattatgcactttaggttgttcttacaagttagattattctacttttggtgttatcatgttatataattcccagattgaaatgaaaattgcatgaaatgtaatatatattatactataatcctcatcatatatttgttactgtatttttggataggtgattacttttgatgttgtcatgttagatttttttcaaattaatttttatttttatgttgtttgatcatcttaccgtactaaagcctcactatccagttttctaaattagaagtgagttattttatcaacaaaaacattctagaaattagatgcatatgcaatttttttaaactgaaatgtccatgagagcaggaaggaggtagtatatcgtaaccaggccgaggctagcgcccatcacttatgatggatgcttcttctgcaggattcactaatgttgaaagttactgctttcatgcgttctaagtttcaaagcgaagtttgatgacatgtcttgctcgccaaaacattgaagaactttctgcatatcatctgtattaataaaatggttctactcaactcagttaatgcaaaggtgcatagtgacaaaatgaaatttataaacgtatgatataattacgacaaactttgggatgcgactgtattctaaatgtttttggcacaacagattcggctaataatataacaaattactgtatctacatagcacatatataatttctaatgcagaatcttcaaatgtgtacaaacccacttgatcggaaaagaaaaagtataggtaccaacatattatctgtcaacttattgttaataataattaattattatattttaaatacatatataaagagacacatccagaaaatatatctataaagacacttctattaaacacagccatgaaaagacatttttattagacacatccacaaagacacagaagttggcaaaaatgctgttggtaatgtagcgagaccgatctgaaaaatagtagaatgataaacattaaaccccaaacatttcacacatataatttattgtttaacattttttgtttggtgactattgtctaacatttaaattaaataataattttcttcttaatatttaaaatattttatttttgtgttaaacagaaattttcacaaatcttcttttaaaagtaaaatggatatttttgaaacaaaaattttgattctgatcaaataattaaattattatttaacaacttaccactatcaatctcaaataaagataataaaaattcattttaccaagaatcctgccaccttctcacttaagatttgtatcccttcatttgatatcaatgggtagaaactagaaaggattggtccaggaatcacaaaatttttaatgtctagttgcctcaatggagtcagtcaaatccctaatcctctaaaacaaaccgtagatggcaaggctttgactccgtcaagaaaggcaacaaacccaacaacttgattccaaaggagcgctccaacgaaaattaaggcatctagtaccaaacttccaaacgtcatcgtctcacaagccttaaacgacacgtcatcctaacacgtgttcacatcttattggttatatcttcatcaacttcgctataaattttgcacaaacccaaacccttcttcgcaattcattttcaatcaaactagatttctctcttcctctcaggatttctcgttctctcgcaatacctccctctcaaggtaatttcgattgttcttcttggcctgttctttcttattgtaaatatatacctgtatcgctaggtgtgttctcgataatttcgagtttttgtccccttaatgaagattctggagtctagatctataagtaggatcaatttgataagttcttggttttttttgaaattatgttgattttatgatcaatttcaaatggattttgaaagttctttttttagggtttcgatctactttagcttcgattgatatgttcatggaccgaattatttgccgatcttatttttagcctgttattgaaacgtgtttctgattacatcatcgctttatggttaatagttcctgtaatttcccggagcaagtttgattaaatcttcaattttgtgtttgtag

SEQ ID NO: 13 - DNA, 2465 bp, synthetic construct
  source 1..2465: mol_type = other DNA
gcctactctttgggcaaccatcaattctatgcccatacttaccgcaaccaaagcagataagatggagaccttcgtactctactttaaactcctttccgagaacttttatagctgggacaagctggtttcgcagatccacctccacacataagcgggcaaactttcctctagagtgtatagaagtagtttgatcaattttcaacattgttctgatctttccacctgcccgccataggaagtgataggtgtagagttcaaccggcagctccggaattctcacccacaccgctaagttttgcatcgcatcctctgacggttgaaacaacggtctccatctctgtactaacaggtagtgtcctgccaactgtcacggcccttcaaagagcgcgtgtttatagtcaccttcgttgtcaaagcgcaccaggaagaactctccaggcagatcaattaccttcacatccccgttctcagaccaaaaacggtaaacccacgagtccatagatcgaaatccaatattcttaccgagtaccttgacaataagagaaagcttccatggacgacaccattcgtcatgccaaactcacctcatcaacctctggctttggattgaagggagctgctctgccggcatcgtcttcagagacctcctccaacatatagtcctccgctaccatctgaacaatttcctccggtgaaagagacgttctcgcacctcccatcaagagtctattcttataagagacttcatattctgcttccgccatttgactttagctttgacgaggtcactcttttttcaaaaccggatggtattactctcaaaacttagggaaccattcacaggttgcagatccatgtccgctgatcttccgttctcctctcgtctagtatcacttcatccccgttaggtttgaccttcttagtgctgcggttcatcaagtctttctcttgaaaagtcgtttcatcgggcgccgtcactctctcactcatttagaattttttgatgtactctctatctttcttttctatttttagtgcatattttaatatagttcttgatcttaatatatatatatatatagtttattgatctttttaattaactattagattcttattagactgaatctaaaatttactattcgagtttatttatttgtagatcaaatttgattcgataaacttattttatatatctacaaattttatatcaaatacaaacaaatacaacagatattatgaatcgaattcagatttgtccaaccttatatagaagataactcctctaaaatcaattggtgatgtaataagaattctccttttcattttgcccattgaattatgtatcaccatttgggaatcagattttaattttttatatcttaaaccatatagcagcaattccccaatgtacgccatttttgggttactcatcctaatgggtcactactttttttttggtaaggaatgggtcactagttttgtaccctctatttttttcagggctggcccgttaattgctaaaacaaaaaatatgaggctggaagatccaagatttgaaccaaacaaaaaatgaaggtaaagaaaaagaagctccgagattattgtggaaccatatatgattaacggactctgttattaattctgttaaaagtcattctaattctaatctttgcttatcgtcgaggttaacattaacacgtggcacatttttattcgttgcttcaacgatattttcgtcattaagtcggcacctcaaagctataaaattcaactctttcttccaactctcgttatcgtacgcaactcgcaattggaaatacggaaaagttctttgatcgcaaaaacctaatcttcttgattctgttcatcgcgatctttgtttcaaggtaagtcttcggattctggctagctgctttaattggttgctattatctgattatagagggcgtagtttgatgattgctttaggttattgatgattattgcttttgcatttgcttatttttttgctttgatttgtttccaatttaggagaagtgtgttagttcttactgattttctttctttcatttaaccttgtttccgatccgatcattttttaatacgaactcggaaattttgattaggttatgcttctcgatatgtttatgctttgtgatttaatctttcttaaacgagtcacgtgtttagtgtctgttgttggaagattggtgccaagatatgttttttatgtgttgaggttcatgattagggtttctgtatacaaactttcatagtactgaatccgtgagtctgatgatcggtttgcggtttttggttgctttgtcaaagctgtagaaagtaatggtttctgaactggttgccgttatgattttgttgagattaggtttgtgatttaattttcagtttgatgtccaagtactctcggtggttgaaaattttgtggcttttgttttgaacag

SEQ ID NO: 14 - DNA, 2507 bp, Arachis hypogaea
  source 1..2507: mol_type = genomic DNA
catgattcgctcatacgaggtacgtatccttgtcatttgtcagacacaagaaagaatcccgaatatttgtataattgatctcgtttcatttcagttgggagtgctctttctaccaacgatgcttaaaagggctggtggtggattttcatgtaccagtaatgttacacaaactgaggtagcttatttgatatggtttggtgctaggaagggaaaagaactccgatgctgacatttaagaattacaggataaatatgaagtgaagaaagcaaaactagttagcctaagtgggctgagaagggagcaaacacactcaacatctcaactgaccattccgttacctgttccttacgagctccctccccaaccttattcctcacaaggtcagtatttctcacatcacatgttaaaaagtagttgttatgtatgtcttctaattattgttctctcatgcagatgttccctggtcctgggatcggcgatatagtaagaaagatgtatatggccaagtttggccacgcacgtaagcttgctacttgctgttgctgctctgctctaaatatacatagtaccattcactttcctccttagcaccatgccagcattgagttatctatgacaaaggtcgctcatttttcattttttctctcttttcattagtagtgtttattgttcaatatctatcacctaaaagaaaaaagggttcaatacaattttaaattgtaattacttgaaagtgatcaaaggttagataactaagtactagaaaattatgtgggaactttatcgatggggagttgccaggggaatattcttaatgatttatgatttagctaaaataacgccttagctctttctctcttcgggtttctctccccgggggtacaaaagagtgaatatttatgaagtttaatttattcacattataagtaagtggaaaattgaacattaaacttacatttctgttgactttgactagtcaatgctaagtcgcattaccaattgatagttgtgggagaaaattttcaatttaatagtaccttttaattgtcggtaaaatttgaaatattctattttgatcattcaattgaattaaaagcctttaatttatgctaatgggttataattcaaatgatatagtttttttattcttatctaggtgtttttagtttgagtctcatttttaatttaaaaaataaaaaaataaaaacctataaatatagtatttctcaattttttcctccaaattattttttggtgataaacgactttaaaaaaaaaagaaacagaagtggattcactcaatgttctttccagttgattatccgatccgtttctttcaagttagatcataattagataatagataataatgtgcaactgtggataagcttgaaaactaaatgccggttttgcgttcctgattaataatcctcaaagaaagagtgatatagagagtagagagagcaacacgccaacatctttaattttgtaaaaagaattatccaccaaataaaaaatagaatctttctttgcagcctcagctgccgattaatggacagtggatcccaccgtgataataaaatctataaaagcccacttgtctatttttttattttgatttttgatttggtataccgtatacccctttctcagcccacttgccaattactacaacttgtgtgggcccaaatattatgttaaggatccacttataataagataacatctattaccacaaaaaaagagataataacaccgtggcggtcaatacgagaagagcgtttgcgtggtttgttgtttgtttgtgatggggaggtctgaggtgccttaactccaaccaacgccttccaatttccaacccttcacgtctggccaaacattgccgtgtctatagtgtcaattgacacctcaaccgtacacgtgtcgcaaactgataaggtaaatggtcaccctctttctgttgcgtaaataaggcggacacctctttatgctataaaggaacctccagaccccattgtttcttcacaattctcattttcattctctctttgttgtccgaaatccttcaaggtaccttcctcctctttctctttgcttttgattctggttccctttagttacatctgtagggttttggcatttcttcttaatcaccaatattatcggttttagattcgttatattatgtcttttgttgcctcgttccttggaaattttctctagatctgtttatactgcaatgatttatgatatttaattgttaaatctgatcggttttgatcacgcttttaaactgcaatttttagttgctttctgaaagatcgaaggcctttgtttcggatcgtaaattttctgaaaaaatctgagatcaattgtaccaattttgattataggttttttttttttttccgttgttgatcctgattcatctgtttatgacgatgcgaattttaatgtacgtttccttaactagtttcttatttaattgttgttaacgatgatgcag

SEQ ID NO: 15 - DNA, 2480 bp, Cicer arietinum
  source 1..2480: mol_type = genomic DNA
ccaatgtcatattcgaggtagatcatactttttaaataaactagactaaaacttttattacacaatcctttttctcatatttaagatcaaattaaataaaaatatttaaaattaaatgctaagcttatattctctcacttctaaatccttaatttttggagcggaactaaatttggcttatgatcatttttattcccctaccttcctctccgtccaataaaaaaaaatagatattttttctagaataaaatataaacaacaataattttaaggaaaaatatatctagatataaaattttagttttagatttggtcgttttaaaaaaaattaatttgaaaaaaaaatgtataccagcgtaaattaattttacattgatgtcaatataaataaaatatatgattttttttgaaagtgataacaactgttgttggagttagaaattattcgagagatcaactaactcttttcaatttgatttcgttttttttaaacgattacattgaagaatgaactatgtccctcaattataaatatcctaataacgttaaagagtgttaaagtacgtcgcgtttatctttaaactactaaactaatgaatatacaaagtaaaataaaatgataaagagatagatatgcataaaaacttgtttgattaattatatgtctcgaataatttacaagtattgatatttatagacagatgctagtcctactggccataagacgtaacctacaaaaatatagctttactcggctattatgtggataatcaatgataattcttgcttatgggaatttttgatgctccacgatcggttcatatttcctatcttcacaacatgtctcgttgtcaatactcgcacatgttttgttgtgcctctgtgttgaacgttaatggtactaaaaacgtataacacaaatgcaattggcaaatcatcaaattctattgtgcattaattcattcacatgtctttgcaatctttgtggacctttagagacttgaaaaagatttgacttacccacattcaactgttaattgccattattgactaacaattctttttagttgtatataacaatattcagttatatataacatggttgatttatattatatgatattataaaattattttacattgcaaatataatcattatactcttttatgatttagactaaatacattaattttgttgataataagatacaataatttttcaaaacaaaattgtatgtttatatttatggtattctagaaaacctgaacatagttgaaaggggatatactaatatggactagcaattggcgtagtcacaaatgggtacaagttgggtagtcaccgcactctggagggtcatattaatgataagcgatgtactgactacacaactacatgcatgtagttgtaattgcactatattcaatctcgtattattactgtcatcaattacatgcaattaaaaatgttttgcactgataatgttaatactactaaattaaatattttattttagtttaaatttaaaaactcacaattagatgaaattataatatagttataaacagacttaattatttttaaattataattttaaatttgtatcattcgatttgtttggacggaaaaaaattggtgaatgcaagatgcaatccttatccatcgtatcgtcatcagttgccgaaaacgattttgactctgattctagaagttgccgtttcataaaggtgggcaccttatagtatgtccacgtgtcatacattgattgactgagtggctgttgccgatattttttcactataaaatgcaaagcccggtaaaccaattcttcaagtagctacagaaagctttcgtttcattgcagtgaaaagaaaattcctagagcacaacaccctttcactttcaaggtataatctacttttcttctcctttccattaagacttcgatcattagggttttgtattctctcgtttaatcggtctttggatttgtcaactttgaatttgatatagctttttcgacatcgattgttaggtttgtgttatctttttaatccatcattcacctgtttgcctttatttttaatctggtttatgtgatttgactgttacgataatcactctagctgagatctatgtgaaaacttttcttgattaatttaattttgtattaatcaaaaggttcaaaacgatcttaagaattggattatgtattgtatgattttataatctcctatagttagattagtcctgggacgttgatacgactgattcattcatttatcggacacaattttttgtgtaattttgtactttgatgagaagtatgtttttaaaacgtttttctgtaaatataactcattatattggctgatcttctacttccagtcctctaacacgtagtcttttaactctgttgtagctataatgaacaattgttgattgattaatttgtttttgtttgttatggataatatag

SEQ ID NO: 16 - DNA, 2366 bp, Cicer arietinum
  source 1..2366: mol_type = genomic DNA
gatatgacacgagaaaactaatttacaccggtaaatagcatgacatattaattgatatacatattttttatagaatttaaatatttattaaactcttttaaataaataaaaaacaacaaaatcatcattgtgataaaatttataattttatatttattcctatacaaaaatttcaatgtaatatatgcagaaactttaatgatataacgcttttataaatttttttaacacatataaacatatactacatactaccccatttgctataatattatcatacatatatcataaggaaaactaagaaggcatagataggattattagataatcagaatatattatatgttttagttaaaactctaaagtattgaatgtcttaaatttgaactttaaaaattcaactacattaaaatcattgaatataattaaaaaccttataatcacttgcgatattttgtatacataaaaataaggattatgtaagaccaataaacaaaaaagactatgttaacaaatattttaaatatatttattaagaaacaaaaaaaaattaaatgaatatttttattacaatgataaaatttaaattacataacttttaatatttttctcaacatttaaaattttaatatttctctaaaaaacaatatcttagagttagacttataaatttatattattctttgttgcttgtcctttagatgatatacgaagcaaaaacttatatcgtataactcaaaggtttttatatatttctttaattactgaagttctacttttatcctcgtattctgaaccacgttgtatatccagcacatccaataagcaccaacaattttcttaagcatgtgtgaaatatttaccaaatttgtctccatcgaagagtcatatcagatatctctcaacattgttatttttatgatttaaatatttatttgattatatagatatattattttttatattagtctatataaattttattgtttaaatttaattattataaaattaaagataattaatttttagtttaaaaaaagagttaataaattgcaggataagtgaatttataaatacacataaatatattttttatgaaatattaaaaattaaaacattttataatattaattaattatttaataattagtattattttataaaaaaatacaaataaaaattaaaagaatattttaaaagtgttttaaaacttcgttattcttttatattaatttgatattaagaactaaaaccatttataaaatttaattcaaaattttttatagaaactaaaataaaaaaattatattcataaaaattaaatatatattttttaaatatattttactataaatacaaattttgtttttttactcataaaactcgaacttaataccatatcgaaccaatagtatgatagtagaaccaaaaaattaatttatttaacattgtaaaaaaaaatacaatttatttaaccattctaatattctctttgttgaatttaatataattttcatagcagtcggccttccaacggtggagtggagttcatattgtaaaacagacgacgtgggcccatacgaaataagtgacccaataaagtaacgccgtggtaacgattaatccagattccagtagaaggttgcattcgactccgtcaagattaaacgccaactaccatttcccaattgcaattaataatttccgagaaagaccgccctgaacgcattggataccaaaactaagccagatacaacaagcaacccaacaactagcaccgaatgacatataatcaagacacgtgtcacaattctagtggttacgtttttgacccttttgctataaattaaccccaatgctcccatacaaatcgcagtttcatttctattgaattcatttctttgagccctatctacttctcaacaaatcctcaaggtattttccatttcttttctgattttcgattcgggttttcgtctttcctttcgtagatctgttagaaatcgattagacagagtttatataagcttaatttattagaaatgatctcagatctgtttctaatatatattctcaataaatctgtttgaattgcatgatcgtcaattaggtttgttgtatctggtttatgaacaggttttgcatgaacggatctgataacgtttaatttgaaatgcttcatattcgtttgttatacactgtttttaatatgtattagagtcaattcaatttctggtgtttgattttagtttatttgttgttttgatctcaagctgttttggtctgttgttatagattcataatccagtttctaattcgatttctggcgtttgattctgatttatttgttgctgctacttccacag

SEQ ID NO: 17 - DNA, 2445 bp, Lupinus albus
  source 1..2445: mol_type = genomic DNA
cagcagtagcatcggcgaaaattcgcagatcagaatgggatgggccttcgaccgcgtctcgaaagtcaggattcttttcagcattgatgagttcaaagcttagtaaagaattgcaatcacctgagaaggcaaacactaaagtggatataaactcggctgtttatcatggtggccctcctgtgccaaaaagggctccaaaaatgctgagctttgtggacataaaagaaatggttagaggtccagaggatcaacctggatattgggtggtcaccggagcaaagctctgcgcagacggtggcagaatatcaatcaaggcaaagtattcattgttgaatataatatcagaagaatctcttctgtggtgagaatagggacttagttaatgcagttttttttggtctgatgtagatatcttatagagttatagcatatattaaaagaattcaaatttaatgagtgacatctggtgcaaagtgacctgaatatggcaatctgggttggtttttagtagctgatagtcttatgattttgtttttttggcaaaactgacttcaatattgaatagtatttggaaattccaatcacttcaaataagaagtttttgatttgagtgacatgagaaaggacctctcaacctgtatgtttataagtgcattccaactttaggtcccaatacactgaatactcataatgggtgattgaagtttgtaactgaacctggattgaaggatgagaggggatatacttactaccagagtaataaaaaaagtggagtatgtttaaaaaagtgcgttatcttttggagtaaatcaattttgttttatataacttttcatatataggtgcttactatttcttttgttatatttgttttctcatgttttatagtagttgatcacaaatgaaattacacaattaggtgttatctaccaaaacaaatagtcaactaataaaactcaaccgcaaagacccagtcataagtgatttagtaattaattaaatatatttttctatttaagttttttaatgaattttgattttcgtttatatagtctttttttcctacaatttagttttaatctttgttgaagatgtaatatcaccataactaagcatgtcatcaattattatgactcatgttgcacctgaatgttaattagattaagaccaaaattttggatcctaaacaaaatctaagaaatatgaatattgaaattaaatttcgctaaactttacgcctaaaatatatttaaatatttattagtttacatattgaaatacaaaattttaaaatgtaaacgtttattttaaaaatcatttattttaaaaatcatttattttaaacttaacaaataattcagttatatatattaattaaaccttaaaattaaatcataaaaacactaccttacctaatgcaaagaaaatcactttaattagaagttgctggaattttctagaggcaggacaaatctgacttagcgaataagcaaatagattccagacttggcataaatccaaaacaattaacggtccctgatcaacaggattgactgtttccaatttgtccacgtgtcacaacttgattgattgaagttgtccttgccgtaacgaagcctctcaccgttttgctataaaaaccgaaccccaaagcatgatcttcatcgcaaaacacaaatcgcaaaatcgtttgaattctcgtagttgaagaatatatccgagcacgcaatttcaaggtatattttcttctgaggttattattgtttactgttgattcttgttttgatttgtttcaattccttaatctgcgttgttcggtaccggtaaaatcttgatctggaagtttttttatgttctgattcgattattagggctatgagttctttattaattgacggaactgagataatcggatttgtttaggcctttgatcttgatgttctgatatgataattagggttttgttttgattgttttttatcatttagggaattctttcggaacgttggtcatgatttacttttttgaataatcaatccacagacgatcttattgaaatatgtcttcatttatttgccaatttgatgcatgtgagggagctatcttgatatcattatagggtatgcatatattttaccaaattgagatttaattgtgtatttttatgcaaatttctactgtatattttggttcaattgattcattgatcggaaactagttaggaaggttttctttattcagggtttattttctgtgttgtctaaatgtaattttgtaatgaggatttggtccgttgaggatttgtccgtgatattgttgtatgtttgggagaaatttgatcaacaacctttcaaatgattatttatgtagtccttgttatatgtgttcccccattaatggccataataatgagttggtgactgatttgtttgttttttaaattag

SEQ ID NO: 18 - DNA, 2387 bp, Lupinus albus
  source 1..2387: mol_type = genomic DNA
ccaacccaccagcaccaatgctttcgtttttttcaattatttttctactgaactgagtgctgcaaatagaaacataaaatcgacccaaatgaaatccattgcaaaagaattgaactttaaaatcaatacgacttaatattggaatataaaagaaaaattaacggattagtccaatccaaaggcaaaataacagaagttttaaatatataagcaaaaaggaatttgaaatcacaaaatccaaaattgcttaccctgattagtgggtttgatggatcaaataaaaaatcacaatagtacactgccgtgtgtttctgtcgggaaagatgaaggagtgaaggtgtaacatttgtatttctatttcaacactatctccaataatagagaggataattatataaaaattaaaatataataaaatttatatcatttaatttatatttatattacacttttatcttttaaaatatactaaaaaattagacttaataatgtccctctagaaagattatattttgactgcaagtggtagggagtaggcaaattgtattcaaagacctccggtggtggatgaaaatttaatagaatgatgaaaaattacacaaaatagtttttgttatttgtgaaaataggatccagctagcgcagataatgtttacacgtcacacatatatgaggatccattagagaccgtatccaaccgacttgagagttagattatttagtgagaataaaacatttatgtatatatattcatcatttatattttattattatttactctttaaaaataatatttcaaacaaatgatgaatgatatataaatttcatacatacagtataaaaatattatactcttataataaacaatataaaaatatttatatataaaaaatagtatttattttatatatgataattttttttaatataatttgatgttaatactccatttgttcgtttgtaagtctcctcatagactttataataaggttattgtggtggaaggaaggaaatcagaggcttaggagcatctcacactatattttatgattattaagattttcgtttcgttccttctaaaaatgacaatatcaaatatatcataggaagataattaaaatggttcatttttaacaaaaacaaaaatacacaggaagtgatatattgataaaaaaaaagaggattatattaataaaaaatgacaatatataaattatttactaaaagaaggtgaaaaagtttggttgataagtatattatttattgagaataatgtatctcctggtataaggagataaattatttcctccaaccactaaaatcgtgacatgtgtactatttactcatttgctacaaaattatttcttaaactaaacaaaataatatacatatcattcttttttgttgattaaaggaaataaattatcttctaatatcggtacgagaatatatattattctcttatttatttacaaaagaggaagtgtatccccgaaagagatttagggtgttttttatatccaattattagttgtaacttaatatataagaaaagcattaataaatctctgacgtcacgccatagttggcgtgcaaattgacaccttatctgaacacgcgtcttaaaatgagtggctaacacatcacgtccactcattttgcgtaaaaggcggtgacatcattttcctataaaatccaccccaaacccttcttgattcacaattcaattctcaatatctctcatttcttctatcgctcaaatccttcaaggtatttcaatccttcttctcttttgttttctgttattagatctttgattttgtggtaaattatgttttaagttccttatattagggttttcgttgtcttatcatttctaatctcactttcagatctgtgttttatattaattttttatctgtttggattgagtcactttccatttaggttttctctgatctctttaataagctcaagcctatgaattttgagttttttttgtcgaaataatcattatctgaatgagttcatcggattcgtttttgaattttagtttctattaaaaattgagccgatgattttgtttttgcgttctttttatccaaatcatcactacctgagtgaaattccccagctgagttattgaattttagatttcgttaaaaacattttaatttatattatattgttagggttgttacgaatatgattgatgcaaatattcgtcggaatacaaatatattatcctgatgatcgttacggatatgattttaaaatcaatgatttctagatctgttgtaattctaatttagttttggattgtttgcgttgttcattccttttacctgtgtgatgttgatatatatatatgtctgattatttctcag

SEQ ID NO: 19 - DNA, 2505 bp, Lupinus albus
  source 1..2505: mol_type = genomic DNA
gaagtttagtggtaatgacgtgatgagttatggtgtttgatggaagagtaaaaaggaagagagatataaagttaagtgaggtaaaatgtgaaatagaaaaattggaaaaaaataagctaaaaaaaataaatcaacctatgattttggaacttatatttttgggttattttcttagaaaaaaaactgagtttataaacttcatttgattatcaaacatacatagtttatattttcaacttattttccaaaaaataaatttaaactgttgaaaacaaaccatgccaaacaagccaaacaagccataaattttaaaaagtacagttgaatgaaaaatgagtcatgaaaaaaggcttgaaatgaacctcaagatgagataaaatatgtaccattgccaaagaccctacctaattaattaattttatggatccactatttttttcctattttttattatatgcccacaatcatcttgttgacatgtaatcaggctggcagaataaatatttgtctataagtttaagcgcagtacttatcaataccattggcaatatgggcaaagcacattcgacccgtctacccgtatatattgggcggacatgtcatcaaaaatataattatttgcccttaaatgtccacccaatatgttttattcgagtttggcttctctccattacaggagaaggcaatgcatacttaagattaaaaaatgtgtcataaaaaaattcaatgatccaaattttaatattttttaaattattttataaaaaacatctcctctcacatatccagtaccggagagatgtgagaaaatgtgttccatttgttatatatgcatttgtatattgtatattatatatactattcttttaatattttgtaaattatgtattagacatattatatatatatatatatatatatatatatatgagtttatattatgtatatattgttcaatttgtatgattatacatttgtacattagtatgcaaattatatattgtttattttgtatgaagtatatctaaatttgtatatattttttagttttatttagggcgactcacaagcccgcactgcccatgtttaggacgaacaagtcatgatatttcatgtcttatttaaaaacatgcatgcctatatccgccctaatttaagtaaccattgcatgatagaagtagggcgaccaccattactacttatcaatgaggccttatgataaatggattcgtaacttactagattatagacacttaaatattaaaatataaggaagagagaaatttaataatttttaaaactatcttattcaaaaataatttaaaatttagactataaaagaagacaaaaatatttattttattgatacccaccaaattaacttatagatatgataactattataatatacactataccactacctatatatttataaatttattttttaattaagtattatattataataatacattatttattttataattaatttcaatggctaaaatataatcataaatttaacatgtacttcaattttattatttaagtaataattaattcattgttaaaaaattcgttagcgacaaataatagataaagagaatctgtaaacaacccattccccgtctaaaaagaaatgaaggtaggtgggtctaccaacaaacgtattcgtacatcttacgtcacgccaaagttgccgttctaatcttccacattatatgaacacgcgtctcattttgattggttaacacatcatgtccactcctttaacgtaacaaatgtagcaacctccttctataaaattcaccccaaccctttgtttcttcacataattcatttcatttctctcctacttctctcgtttctcttcgatcacagttatccttcaaggtatcttcttccttcctctttcttttgtgttatctctcatcattttccttttcagtctagtttcatttgttaggttttaatgtttgattttcttctattgtattattgaaaaatgattgatttgaaatagatctgtttgaaattctatatattggagttttgaatccaaaatttcattcagatctgtgtttttcattaatcgtttggagtgtatcacttttgatttaggttttctctgataatatttttttaagctgaagcctatgatcttgttcttgtgttctgcgtttttgttcatcgtaataattgtttcctgaatgaatttctccagatccgttattaaattgtagtttccgttggaacaaaacctaatttatatttagtttgttagggttaatcttttgtgtctttaagattgtgattcatttaggtccaattattcgttgaaacacaagtatgttatcctaatcatttttgtggatatggattttttagttttgctttaaacctcaatgaatttttgtaatgttatttttgtttttgatttaaatcgttattgattctttttagaggcttgctttttatttatctgtttctatgattatgcag

SEQ ID NO: 20 - DNA, 2496 bp, Lupinus albus
  source 1..2496: mol_type = genomic DNA
gttgctagacgtataggcatgttatatggttcattggtcctcatcttatgggtaaagtttctagacataaagctttattttatgagatattaaattgactatgacattaggttttagtattattgaggttccattgttgtgaaagattcattgttaaatgaattagtgttgtcgaataaagtgtgtatggacaatgagtagtaaagttcgatcccaaaatctttattccaacattttatattgaagataagcatggaacacaacaatccaaagccaagcactcaggacagccaagccatgaccaagtcatatgaccgagaagaccaggccatgaccaagtcatatgaccgagaagaccaggccatgaccaagccagatgaccaagaagaccaggccatgaccaagccagatgaccgagaagaccaggctatgaccaagctagatgaccgagcagaccaggccatgaccaagctagatgattgagacaaagaaagacctatactaaaccatgtatatggatatttttatgactttgtccccattaatttgctttgtgttgtagattgtagcaaatatattaaggatatatgaacttagatgaaaaatagttgagagataaaaatacaaatgatttaatcattcaattaatggcaccataactatatccagatataatagttggagaatgcaattacaaacttttagagtataaggatatatttcaaattctcaaataattcagagttttattaaaacttaaaattcacacctcaaaataaaattattaccttacctatcataaagattaaagaaaatcattatcctcttaaaacagtttactaagatgcatagtgttgattttttttaaaggatcacgttaatttcatatatcaaatattttttccacaaaaacacccttttatgcttattgcatcatgttggaacaaatgagcatttcaactatttgtaatgttgaaaaaaactccagaatctaatattgggtgaaacacttcaaaattatttataattaattattgtattcttggaatatgttgaagggatacaataattttgtggtgggagaggaggtacacaatcaaaatttctccatacacacgtaagattgacctaaacaatcttttataagtgtcatgtaatccaaatctcatctaatttatttttcacttaaagtcttagttcaaaatgctaaaatactctcaattgaaaaatgtgatagtgtaaattagcaaaccccttttgaaatgaataaaaatagaataatagtgatataatctaaatctcaacacatgttagttttttttctaataagatggtcggagaataaattattacttttataaagaaaatcacatttactagaaggttctagaattatattcttggccaataaacaatatattccaaacttgaccaaatacaattacttggctttgaccgttacttttcgtccacgtgtaacaatctgattggtttaagttgtggttgccttaatgaaccaattcaccgttttgctataaaaacgaaaccccaacgtagaataatcatcacaaatttcaaaattgaatttctgaagattaagagaaaaccctagcacactttttcaaggtatgttatgttttcttcttttcgatttcgcgattattcttcacttttgtattctatatgtttattattctattgtgtttgattcaattttttgcattctttatattaattgatggaaaactgataattggatttgataagccattggatcttgatgtttgattcaatgatcacggtttctggtttgagatttttctgttatgagtttcttttgaaacttcgttcatcgttttgatttagtgaattatcataccagattaggtctttatgaaatgtttcttatttgatatggcaatatgattcctatgatgaaactgtttgagatctgtgatgaaggtattgacacacgttggtttattatctactttgggttcaattgattcatttcttatctagttgtataaatgaaattttatgataaggatttgggtttgttttttattattcaaatttgtgatcaataaattgttataaggtttgaagggctactttttgaattacggatacaaatactgtaatccaaattctgcgcattccaagaatattctttaatattctattttttcagctattaaaccctgtttattttaccaagttcaatcttagtaggattaggaactatttatatgttttgatggaacaacaaattggtttcagacatctgtttatgtagccatatatgtgtgtatcgatggacattccagtaggaccagacaaatttgacgattaaaaaaaaaactttaattaaaaatctgccccattgattttttttaaatgacacatgtcacaatctctagttgtaccggagaggattcaaattccttaacaaaacataggttactgacttatttgtttatttgctatatgcag

SEQ ID NO: 21 - DNA, 2424 bp, Lotus japonicus
  source 1..2424: mol_type = genomic DNA
gcccatacccacttataagcacttttagtcttatttcatttagtttctcaaatattcttttttaataaggacgcatttggataaacaacttaatgtttacgccaataagggcaaatcactattcctaagctaataagttcaaaataggctcaaagaaatgatcatcataagctatttgcataaactcaaatgaagattccaattttttttatagacaaatgttagtcgttaattgctaataaattagttcctttgtcgggatttggaccctgacccttcaccctgcaaatctctttatttcccttagctcaccaaccctcccccgaagattccaaattctgttgcatgtcttcaaaaaacttcccaagtaacatatccacccaaataaaaaccaaatttgacagagcatctgagtaacctgagaaattgaagggtggcaggaagcagcatgtgaagcagcttgatggagagaaggaccagagccagccaagaggagagaaacggaggagccgctatcaggaagacacgtggtggccgtgagcgcagagagagaaggagctctgatggcaccaccgtcgtgctccgccacaaccagcgtgctaccctaaatcgaattcaacaacaatggcacagtgaataataaatcgcagaaagaaaatcgaaaatgggagcgagagagagagtgtgatttgttacgagtcgagtagagaaaatggagaagcgaggagatgaggggaacaagaaggagcgtttgttcttcattgcttcgaggatcgtgcgagacgccattgtttgtttcttcggaactgaagacgatgggttgatcttgatgttgaattgaaacggtgccgtatcggagtcaacagttgcaaatttgctgattcagctttaggaaagactgaggattagaagaagatgatgatgatgatgatgatgatgataatgacaaggatgatagtgacgatgacgtgttcttatgtttaatgttttactctcactgctggtagatgcatgtgggcgtgggcgtgggcgtgggcgtgggcgtgtgttgtctatctccttattatccaaaaaaattcaatatagggttgatagttgagttgcttaaggtggcaaagagttcctatagttaaagaatgaaaagggctagcaacacattcttttacagtttcttttcaacactatatcattgattgattgaaatttatgtaggttcaataaaaatatgggtcacactttcaatttaatgagactcacatgaattttaatttcttaattttgaaaaaatttggatcctctcaaactaaatttctctcgatctttcttaatccatgaccatgaacggttgattttaaatctaacttattaaaaaaaatataatttaaattttacctcatgatgtgaactaagtgaaacttttttttattcatctgaaaattaacaaacaaactaaaaaaactcaaggaactaacacaatatctctcattaccagggtctcaacctcccatggaggctcctccaacacacaaaaatcgacgaatgcagaattagctccacacatggtaagaaaatctgcaggggcatcacaatcacttggcaagaaacttttgtagactgtttgatttaaaatttactcattagaaaaaaaaaaaattattagggctcaaatcatgattgaattgagagagatttggatcaaaattcattgagagagacggcttaatatgcaagcaatgacataattattttaaacagaatataatagcatcttctagagcattcaagaagagtgacctgggtcagcttatgtacacgtggcaagtctttactcgtcaaacacaaggatagtttcgtcattgggtcaaaaaataaaactataaataagctcctccttgccctctctattcatcgacattctcattcgaattgaatcagttcatcaaaacctaattcctcttctctctatttctcctaccaaggtgcgtcttcgtctattttgaattcatcaaattgcgactctggtttcattatcttgaattctgattcatcccaaatttatggtttaatatttgtagttctaataggttacaatccgattagggtttgatggattagggatttgtttgttataatctgattagggtttgataattgatcctgcttatgttttcaatgtcatgagagattcactagtttctgattttgtgatctattcattatgtgtgaattcgttccccaaatttgtggttttaatgtttatggtctcaataggtgcttatgattttgatggatttagggtttatattttgatttctatggtgtagttgctgtgttttttatgattttgatggatttagggtttatattttgatttctatgatgtggttgctgttttttgatgttttagtggtttgatgttatcag

SEQ ID NO: 22 - DNA, 2274 bp, synthetic construct
  source 1..2274: mol_type = other DNA
gacggaggggtactcgctacactgttttgtttcattgagggtacaaacatagacgaaaatagatgaggggtgtcttttacacaaacatgaaacatcaggggcaacaaatgcttttaagcctaaaattaataaaatcatatctaagatcaacaatatctcagcaaagattaaatgggttgactctattatctattggaaaagaaactttaaaataatcgattatgataatttaacaaacgattttgaatctcaaaaagcccgaaaataaattttatttaaattatttatattaaaaaattaattttataaaggcctaattcttagagttcgcttttggcctcaatattggttgagacggccctgcttttctgcaaaaggattttttttgtgttcaataatattttatgcatacttctaatatacttggattaaaaagaaaattgagagataagaagagaaataagtggaaatgagataaagggagatgataagtgcatgggttgggatggtcttattgggatgggcatgacaagtagatttatggcttccaagcgtttgaagttggcaaaaattcgttcatttcagaggctagcgtgttatgtgaaggtctctctcttgtttggaactgcaagtatagaaatgccatttgtgaaggggttgtgcaaagttactttcttccattgagacctaaagagcttgagcttgatctggtattcaccttccacgactactaccaccaccaccaccatcccttccaccttccaccaccgcctccaccactacaaccattgttaccatcacttccaacaccacttttgccgctgcaacctcctaacccaccactaccactacagccgccgcatccatccaccatcgccaccacctccttctaccgcagtcacgtcactaccatcatcatcatcatcactttcaacaccactgctgctgccaccctaaccaccactcagcatcaccattgtcacctatgcagccaccatcacaaccacatcctccatcaccatatcgccattatcgcagctacatcggctgccaccgtcattttatcgctcactaacaccatcttcatttttattatacatatcattattcaatatctcacaaatcctctgaattgatttttttttattatttaatcattttttatgtgtttaaatattaaaaagattgaattcaatgattaaatttgagagtatcttttatttttaacaataatctgagaggatatcgtcctttcttgatacccaataaaaaaaagttgccgaaacggactccaataccaacgttggcgtgcaaatagagtttgaccgtctaccatcttgtccacgcgtctcaacatgattggctgagctggcgttgccgtaagaaatcctcatatcgttctgctataaaaaccaagcaccatatacaacattcttcattcacaaatcacaatagcaaagcaacaaacatctctctgagtctttgcagagaaagaaagaaagaaagaaagaaaaatccctcatctctcaccgtttcaaggtatccacactttctactctttctctctttcatgataagcttcgattcgatttttagggttttgtttgttcgtcgttaatcgaatcgaatttaattggcgttgggcatgattttactgttttattattcaatcaagattagtactttgaaacttttgtttatgaattagccatgggtgttagacttttctatctttggtattgattcaattgttatcgtaaacgagtttaatcgataatcagggtttgaattcgatcgttagggttttgtatttattattaatcgatcatcagaaaaaccacttttattttgatttggattcttaggtgtccctaaaattcttttgaattgaattagtttacatgattttactgtcttattaatgaatcaagatgatggctttctgaaacttttcttgatttacttagccaatttccttagaataattccatgattgaaaattatgttgagatttcagatttaggatatgtattcagtatccaataattgtttagtgtcttaacctaggtttatatttgttttgggtttttacttctttctttctgggctttttgcttctgtatgtgttgttatttccctgtaatttagcatctagcattgtcaccggatagagtttttgtaaaattgatgtacttgctgatccttttcagttctgttgaacataatgaataattggtgattgacacatttgccttttttgttttgatgatgcag

SEQ ID NO: 23 - DNA, 2294 bp, Lotus japonicus
  source 1..2294: mol_type = genomic DNA
ctgctctctgggagtctgtcaacagaacaccaaagatgactggttttgctattgtttaattctttcaatgaagtacgtacttagtaataagcttcggtcttgcggcaattgcatgtttggtagtggttctgtgcacctgtaattcgataatgttaagtagattttataaaaacggtttattatcagagttttgacttgcatgtaaagatatctgaaatgcctcctccttttgctctttttgtgccatggttcgtactgcttgggcttaaggaaggtgcatggaacaagtgggtgattaggatcattgacactaagattatacctgaagttaggagggaatggaagagggtagttgttaactagcaagttttctttctgctatttttgtgtgtgctccttatccattttgatattaactccctatcatctgtgttcaatgaaaacatcttcaacttcttccttttctcatcacatacatcactataaatatagaggtgacatactcttaatctcctcacaattccgttctcattcatgcgtggtgtggggctagaagtggtggtgccctccaaatcgtcttcagcttccccttccatactttcatggtgtggatgcaccccaactgatataaatactccctttgtcctacgataagtctatgagttttttaatatgaaaatataagtcactgcgcattttctataaagcactatttatatttttctatgtttacccaagttattattcatcattataaaaaaaattacagctttcacttattatatttttctataattatctctcttaagtggatttcacattccaatattattatttggtctttataatatatgtgaaaaagttttgcatatgcacagaatgtagagagataagaataccaatacttctttttatcaactcatgcgaattacttttagccaatccaagtgcaatgaaggatgaagggcacaatgaaaatgactttccatcggagataatcaaactgcaaattacaagtttgatttaaataaatttctacatgtaacatgatgatgatggaataagattggtttccaaatcttcaaaacaatgaagatccacgtttgaggctctgatcctcgtttgaagcttagacccacgtttgataattggatcaaaacacaccctaatatggagagaggattttgaggaaataattaattgaattacttgtattattgataaagtaatttagataagttgttagtacaacttattgcaactatggatagacaaaaatcacttattttaaggggagaagtatataacaacttattataaaatttctgagcagccggcctcctccaatcaatcatagagagtggagcccattctggaaaaccaaggaacccccaacttgcactggtgcggtggcccaattcaaaaaaaacggggcccagcaaagtaacgccgttgtaacgatttatcaatccaaactcaaaaggcggcgagatgcgtttaactccgtgaaattaacaaaccgccaacaacttgcaatttgcaactaccgtttccaagaagaactcaaccacacaacgtatcctatcccaaaccacacgcaactagtgacgcgtcataaggacacgtgtcacaatttgactggttaataatttcaccgcttttgctataaattacctccaatccccttagcttcttcacaattcagttcccaaccctaacaattcttgttcatatcgcttctctctactttcaaggtatgatccaatttctctcttctttctctgtaatcctttcgttgagttttgtttccgatcaatcataggtagttttcttgtttcgaagcatgagatctaggaattttttgtgattttccaaaattgagatcggtttgaaattgaattttacagcttgaatctcagatcttgttttatcaatgttttcgatggcttgcgatgtagatctatgataattgtggttcagttttgttaggaatcgatttcggtttagcaattgcagattaattagggtttccaattgaattcttcagatccgttatggaattatgtcaaataatttattcaaattggaaattattgttagatccactcttaatctgtttgatccaagcttcaattagggttttcacttgtttcaatttcttgttatggattctgatttatctgttgatgttagatccactcttaatctgtttgatccaagcgttaattagggttttcacttgtttcaatttcgtgtgttggattctgatttatctgttgttgatgtgattacag

SEQ ID NO: 24 - DNA, 2365 bp, synthetic construct
  source 1..2365: mol_type = other DNA
cgtaatgcgtgtatgtgttccatagtttgaagcgaagattgatatgagagatctaacaaagcataaatttcaaaagtttttgtcaataatgactcagtgttgtattgtagagtcaataatgtgaccaattctcttttttatcagaaaaaaatgaccagttcctttctctttctactctagctatttaataatactattaggtaggggagacgaatccaaggatttaaattatttgattaatgcttaccattaatttaatgatatcgccttatataaatccaaatagaaatgacaataaaaattgaaaagttagagttacggaacgaaataaattcaaaacagtaataatagaaacttgaacaaaggaaggcgccacaaacaaagtttgataagcctaggataacatggattcttgataagatcgcaagatgcagggaaaaaccctagcagaaaaacactctttaaaattgtcaaaatattcgtcctctattctgagttccaataatatataaattacaaatttagctagaagtttttttttttgtttgtaagaaaataagacgatactataatccttatattctaagaatccttatattctaagaatgataatttgtttcactgattagattaatactctgaatggatatttttttaagattaaggtgaaaatatgactccaaataagttagaataaggtcccaattatggcatgtgtttgcgctttccgccataaaattaaaaaatttgtaaagggcaacaagtagagccacttgtatgtattttgtttccatattttgtttccatattggcaataatgcttttatataataaaaataaagtaaaagacattattgatcagagggtcttatttaattgcatcttagtccacttattcatcattgtacccatcttagccctttctttatcaccgatatctcactcctccacttcacattgcaaatatccaaactagtataataattcatactttgtaatagtgtctacttagtttaatagtttttaatgtattttcctgtattatataattgagaatatttaaaagattgtgtttgtatttatatatattattttgatgaaagagtatactttgttcttgttacattactagaattatcactaatacattgattaataatgcttaaaaaacttgcgcagcatggtgtcacgtgcatggtctcttaaattaaggaaaagcatagattcctttgagcatatttttattgggtaaagtgtaatgggtatgttggtcatcttcattttcattcataatcttatcttatgcatccctcacaaaacaaacccttgtttttcgctacttcatcgtcgtgatgcccattgtatcatagattaattaagttgatagattaatttagtgcgtttttataaaacaacttgcatctttcacttttttgagtatcattgctaaagcaacttgttaaacaatgttaattaataaaaaaacttgttaaaggatgattgtaatccaaaagggatgcatccctatattgtttataaagcgttgcatataatatatatacacactgtggaacagaaaaaaaaatcttagtaagtacgatttacactttcattctaatttctaaaattacaatatttttaatgtatatttgatgcattgttttaccttttatatttcaatactcattttttttctcttttacgattcataaacactattctacatattttacgattaataaaatatatgattaaagttccagcactattctacctatcattctttaaaatgtcatagtagaaaatgaaattaggaagatgcaaactgtgaaatttgagaaataagggtaggaagatagcatgtatgaaattgtagtatcatgggtagagttcgatgttgtatatgtacagaatcatttaccgatgttaatatttaacgacattaatattgaatctgtacaaattaagggtgaataattaatttttattataaaattaagttagttataagacccaatacaagaaaaaaccaacaaaaacttttagtatataacaaagaacacacaagaagggaaagacattgacatattggttaagaatacacttattattgactattctgaatatttttagaacacaaatggaaaaatatgtataaataaagagaagagaagagggtgggttagggatcataaatttaaatttaaagttatttaatatttttgtcctacctaaatgaaaatgaaggttaggaaaaaaaggcaaggcttaaagaagttattaaagaaaagaagcgacactatgtagcattcatgttagtggagatagatataagggatatttatggaaagttggttcaatgaaa

SEQ ID NO: 25 - DNA, 2431 bp, Phaseolus acutifolius
  source 1..2431: mol_type = genomic DNA
cctttagtactgtaccctactcccaaatcagcagcctttacgccaagcaagatcgtcgttgtcctttctccattccgtctccttctttgtagcgaatctctcagggcgccctaaacaaccgagggatagagaagatgaagaataaggatattctaaatatgcgccaccaaaagaaagagaggaccgattgataccaatagcaagatacagtaagtaaacttgttttgttttataaagcgaaaggttgttattttaataatgactatagcgttagcgctttcgttttcaataagtttaggcttgacttattcgaaccgaacaatagcgacttaaagctaaacctatcacgcatgcatttcgattccacaaggaggcacgagtggaatacttgtaatgagccgtgtggcacttactttcggaatgcgcaggggacaaatgccaaagcaaatacaaagaagaagctcttcattcgcttaacacacttgttcgcttgttaattttcatattagttgaaaaacagagaaagtggagagaaaaaaaaatgaaatgagcaaagaaaattttaggatgagttactttgactcaacttttcttaccgataaaactgataagatgctcattagatatacaaataaaaattgagctaaatatttaaaaacaaaaaaaagcacgtaaaaatatatatttaatctgggtattattgcagtattgtcttcataaaagaacaactaactagtacactttcaatggtataaatagagtatattgcatcaattgtatcaacaggcttatagttaaaaaatttagaagtaatgcataaaaaacaaaatgctcccacctaattttttaacgacaaatttgttattaattaagtaacaatgtatttttttcatgcgcaatttaaacgtagatatattgtttcacaaaatatagaattgataataaaaaaataaagattaagtatgtatttcatactctcgtgaaattgattttcgtttttattttaaatttaaatctttctaaatatttataataaaaaaataaatttataataaaaatttgtttattaatataaatatccaacaaatctaaaatttggaggaagaaatataaagattagttattataacattaaagagtaatttacaaaattattaaaaattgaattatttttcagattaaagaagatgattgtaatgatacaatttaatttttttttatcaacaataaataaatcaaattaaatggaatatttgaggggtatcccaacccgtatacaaaaaaaattcttatatatgacttagagcaaaaaccgttagatgacttctctacattcttacacatcccaaagaagattaccctacactacatcagcactttatacaatttaattttaaaagagacaatgatagaaattaaattaagataatatatatatagaggctgcacacaggtaattgcattgactgatacgagaagttcatctaatttataaattattcttattatataacaaattatttagtttattcctaactaatataataatgttaagttcttcgaactataagttagatttcaagctacaatagttgaattttggattggtattcaaataatagactgatcaatttcttaaatagcctaactatgaatgaaaaataaataaaaagacaaagggtgatgacaagttgttttataacatttttactaaatatttaatataatataaatttataatataatttaaactctcaattattttaataactattacataattactctaattaaaaataaataaaacatattttttcttggaaatttcaacgaagatactaatttaagagggaagcatttgtccttaacggagaagcaaaatatagaatgtgccagagcagtgtagaaagaacgtaatccattgcgtatggaatgcttatcgtgatgtgaacaccgccatacacgtggcaagtctttattcgttagtttatggatatttttgtcattgaatcatcgcatccaagctataaataccacccagaattccctcccttcatacatcgtttattccaattctcaatctctcagccctaattccccaatctcgcagtttcttgatcattcattttttcccctttcaaggtatgccatctctccctttctcttcttatgttcactttttttgttttagcttagctcatcgataataacgtattctcgtaccgatcttgctgtttcttttgattgtgttgatagagctgtcttttggattgctattgaattggtttgtattggaatatatatcctgatttttagttggaagttgtttaattttttttttcactttaattttgaggaattagggtttgtggttttattgaagtgcctatttacgtctctgtttttgatgatttagtttgtgattgtacag

SEQ ID NO: 26 - DNA, 2351 bp, Phaseolus acutifolius
  source 1..2351: mol_type = genomic DNA
ggctccttagtaacgtgtgcttttactaagcataaaattatgttttgaacttataaaaaggttataaaagtggttagtttggtagaaaatttttttggataaattttggtctttatgaaaaaattattcttttataaagtgaaggatactagtatactatttttataaatttcataatgactcaaatgagaagtaaaagtttgagtaacacgggaaatgactatcttctattctttagaatctcctagcttaaacaatgactatcactaagtgataagattattagaattttaggtgagtctctaatatttatttatctaagttgttattcttaaaatagtgtactttaagtttaattaaacattataaaatcagtctataaattgaggtttacacttactcatataccataaaatatttttatttctggtagttgtgggatctcaaatacatccccttacgtcaagatcctccaactcatgtgtaagattaacgggtggcctgataaactaataaactctcgttatgatagactttatatgactttaataccatcttaagaagtgaactttaaacctaactcaatcttataaaatcgacttataaaatgaagtttacatccacttatatactatgaaatatttaatctctagttgatgttagattttcaatagtcacaatatatattactcaaacaacaacatattctaacttaaatatattatgactgacttagtaagcatgaacgtaagaataatttgtgttatataaatattataataaacatctaataatatgaagaagtagtttctcatcaagtttaattgtaaatttatcataagtgttacacatataatacaaaactcatatctcaataaactaatattttaattatatttaatttattttttaatttcaaatattattataatgttttttcaaatatatgtgttttatttttgagtattaaaaaatcttttcctttattataaaaggaacatttagaatttcggaaaaaataggctcaaatacgcgttgatatgtgaacagtttaaaataaaatattatattatattacatataagtaaaaaagtctctaataaacagtcgattttttttcttttgttttctttataaagaaattattataaactattaattatcaaataaaatgctaaacgagaatttttgtacaaacaattaaaattacacataaataaaaatattaataaccagacaacttgtcaacatgcagataagatggtgtagcttattatatgaaaactagattaaatgataatgattatacccattactcttaaagttgtctttaataaatcattattaattttttcaataagctatcttttacccttatttaattggtgaatacgtcacctctaggtttcataatttgtttaaagagttaaaacatggataactacaagacatttcctcattagaaaactgaatgataaagtttttcaatcattcatcgttatcgcccataaaaaaaatccaacatgacagctttgcaatagaattcttaattagtaaaataggagccagtgggcccacatgaaactgagaacgctccacttgcaattggttcgggcccaaattgtgaggtggggcccaattcgatgacaccgttgtaacagattcgaccgagcgtctgactccgtcaacattacgaaaatccaaccaccacgcaaattgccctcctccttttcctagaaggtctccccgaaaacgcatccgacaccacatatcacccgtgtcataagcaccaagtaaggacacatggacacgcgtcacagtgtgattggacaggattccaccgctttagctataaaatgacccatgccccctttctctcttcacagttcaattcaaaacaaacttgttcatcctctttcggccgttccctacctctcccttcaaggttcgtccatttttcgtgtttatgttattcattcgttctttgaatatcgttttctcgattaggtcgttgtgttttagatagggtttgcacggtttaatcgatcttgaattcatcgtcagaacattttctttatcttttacaataacgtctggtattgttgattgtgtaatcttatatttggtctaggtatttgttttttgaagaaattgttcagatccgttaacagagcgtttttctttcaagaaattgttcagatctgttaacaaccgccttgtgttgtttgtttgttagctagattatgttttttccatgaaattctccagatctgttcttgagatttacttgattcaattggatagctgtttaattacatcgattgattcgtctctggtttttatctctgcag

SEQ ID NO: 27 - DNA, 2460 bp, Phaseolus lunatus
  source 1..2460: mol_type = genomic DNA
ggattgtagactggtacaagttatcacttctaaccacggaccattgcaccttttttgacgtgttcttaggttccttaggattttgtttttctcttgtcttcaagaaaatgtcttgttctatttattacttttaacactctttttctagtttaataattgtgatgacataaactaagtaggaaaagtaaaaataagcaaaactttcaaattgcttaatgtgtttaaaaaaaagttattttgatttcatgagaatagaactgcattaaaaaattaggcaaattttccttacaccaccataaccctgcacccgatttttcagataaatggggaaaataactttgcgaaatcatgaaaatattttaaatgaaagagggtatagataaaaaaatactttcggactttttttccataatatacttttagatttggattttcaaaatatatttttaaactttgaattttttttaaatgtattttcggattttgtctttcatatttttttaaatatatattttttatttcaaatttatttttttaaaaatagattttaattttcaaattatatttatttttaaaaaaaattacaaagtgaaagttataatgcacgaagaatttgccaaaaatttacaaaatgatttattaaagtgcatgataaataggtcttttggagatatagaaataatatggagataaggtgttttaaagaataatgatttttttcttaaataaaatttgaatagttaatttttttatttacaaattttatacttaaataataaaatattattcgagttataccactcatattctcaaaaaaattattattaattaaaaaattagaataatataaataattttatataagaaaaataaaataaaaatatgtttgataatgtatgatgtttgaatttaaatttgtcattattgttgttgttattatatatatatatatatatatatatattttattttttaattagtgaataaatatttagtagctatgaaactaatatcataaatataaatttgaagttttaaataaattgtaaataccgattgataaattaattttgaggaatgttataaagatcttatattaattaaatgaattttaccgtttatttgtttatgcttatttattattctcaataattgaagcacgcttagttaaaaattaatcaaaagatattataatcaatacttacttaacatgttgaactttccacagtctcatatcaaacatgagtatataataaaacaaaatttatacgctgtcaatgtattttattcaaattaaaagtaaattagtgtaaatttataaaattaaataagtaaaatacaaaagttaccatacatttaatatatacttattttaatatattctaaaattagttttaaacaataaatatttaatttagaaaaataatttgcttatttttattttcttacatagattataaaaaattttaatttaaaataaaattcctttaagatttttgctatcacacatatttaaaaacaacttatcattaaaaaaataaaaaatcacaaaaaaaataaaagagggatcggtgtaaggaaattatatattagaagcaaaataaagaatctaccagagcaatgtagaaagaaacctattatatcttgccgtgtcagttacaactacatcacacgtggcaaataactatttgttaatgcattgatattttggtcatttagtcaggcaaccaagctataaataccactccttcatttcgtctttcttgcatatatcgctctattcccaaatccatatctcaaatcctaatttgcagattttaatctttcgatttgagttttcgtctcaattctcaaggtacctttcccttctctccctttctcttatttgtttaccaaattgcaatatgattagccttagcttattaagttatactttaggtcttcttcgttgatcatattcatgtttcgatcttttccttatgttaatctttgttggtcctttttgtcgtcgctgaaccagaaccaaatatgtcgcttttcttgttctttaatccgtttttatatgctatattttttacggcttttaattaggctttgtggtttaatttattttctcgttttgaaggatctgtggtttgtggtctgtttgattctgtaatctgtttgttgtttgcaaaatttgctgtatactttctcggtttttaactaggggttcgtgattcaatattttttagtcatttctgagggctagagtttatggttgaattagagtttgtggtctgattgattctgtaatccgtttgttgttagcaaaatttcctgtatacttcatcggttcataattagggctttgtgatgcaaagcaaactcttttatcatttgaggcaagagttttgtggttgaactatggctatactacacatgtctatgttattgatgttttagtttctgattgaacag

SEQ ID NO: 28 - DNA, 2383 bp, Phaseolus lunatus
  source 1..2383: mol_type = genomic DNA
gggagcactgttactggacgatttccgcacggaaactccgatttgatcaataattttgagtattggataatcattgagtgatgctccaatagcatatctagaagctagaaagatgaaggacctaaaagcaaaacaagatctctttcagtctaattttggaaactgttatttgcaaagaaacttctgaggatatttgagacttccttcaagcctgaagaagagattttgagaaagtagcgatgaaggaggtgaggatcaatcatattatattgcaagtacacaggagattagcaacaagatgtaattccatgacgacaagatggaatatgtcactggtgtagaaatgattctacggtctttgatatcaaagttcgattatgttgtgtgtgcaattgaagaatccaaatatatagacaagctctcccttgatgaactacaaagttctttggtggtgcatgagcaaaagatgaacagaagttcaagttatgcagtggaggagcaggtgttgaagtttaaattaaaccaaatggaagatcatgttattcactttgtatgaaaaagtgaatgaatttcgaggctagaatatacatgattaacacatcacctcttttaaaaaaacatattttataaataatttaaaataaacactctgcaaaaaaattgattgcattttaattcaaataatatgtaaaaattaataaaaaaaatcataagaataagatatttttgaactagtttcccaaaaaggaaatttaataaattatgttattttcaaattttgattttattattgttattattatgcattaaaatatttaattatttatgtttttaataattataaaattggagtatacatcattttagttgtcatgtattgtattatataaatattttatatctctaaataatacttttcatttcaaatataaattttaaaataattacataaaattcaataaacttatgatatatcgctatatgttttatgttatttgataataatgtaaaagaactatatttctcgacatttaaattgagattaaaaaatagaagaaaataaaacggaatacaagtattttagcttcttttcttgcaatataattttcccaaagctgagctcacgcaagggatccatatctaaacatgcaatgagaaaaccaatatcaaggatatcctcaagaagaaagagacaaaatggtgaattaagagagaaaataaatgaattatttagtcaatatttatattgtctaaatcatcgcctctaattcttttaaaaaaagttcatcaaaccaattgtgatatataaaatattaatttagaggataggaatattttgttaatttaaggctgatataagattatagtatttgaggaaaatgaaatatttaaataaaaagtcctcagatatttgatagaaaaatattgtgtgatctaaaataaaattcattttagaggaaattctaaatactttttaaaaacggaggcggtgatggccaacagaatttcaggagttgttggaaatctcattagttgcctaaaaggactcatgccaaacgttgccgtgcaacacggtttgcttcgcctcgcggtcttagttcatccacgtgtcacaatgctattgaacgagatgccgttgcggtgacgaaacaacgcaacgttttgttataaaagccaagcccacaccacggttctatcataccattccctacataatattcattcttactgtagctgaagaagaaaaccctagcgctccgtttcaaggtacctttggcacctcttctcttttcgattagaattccgattgtagggtttttttttctttcttacaaacgattgccctttgattttgttctggttttttgttcgttgaatcttttcttttttagtacttcgatttgtttcaaattattcgttcgttgaagcgatcgtgagatgtttttatgaaattgtatatttaatttgtcagatcttcgacaagtattctggattgtgtaatcctggaaaatctcttgtaatttaggtttgttcagaggtattttttcgtccgtttcggatatcaatttgactaattgatcggaatacggttttctaagcctcgtacttttataactttaccttttctttgcgggctttgactctgtatgttgtttacggctctacatgtggttttcgtaactagggcatattgatagcttgagagggtttgttgatctacctttttcagttttttatatgtaaatagaattgattgtctaatttcacgttcagtcatttgctttgtgttgtctttttaatctctgtttattgtgattaagaattgtgattaagaaattggtgactgattattttgttattttgattgtattttattatccag

SEQ ID NO: 29 - DNA, 2376 bp, Phaseolus lunatus
  source 1..2376: mol_type = genomic DNA
gcaaccctaggtcgtctaggttcccaaataagcatctaagataaaaaagagacatggagtgaataggtggaagcataaaatgttcaagcatgaatgagaagcatgagaaataagaaagacataaaagtcaacatgtcatttttttattgaaaaagattttttgtaatgaagaaatgtcgtccttcatcctatagtttgagagatttttctctaatgagagaaaataaatatgataaattttcactaaaattacctaaaaataaaattacactttttactggtaataattgttttcacaactttcataaataatatgaaaattttaaaaatatttaatttttgaaattaatttattatacaaaattttcttaaaaaattataaggtaacgtaagaaaaattaaacttaagaattttatgagaattttttatatttttggtggttgagacttatattaagtttattattagcataaaaaaatgtattactaacatacttttttttcttaaatattttaaaattaattagatggtatcattaaaaaattttaaaatatagaaaaatttcacattttaatataaattttttaaatcattgatttcgataagtatcatctgaatttattattgacacctatacttcaactttcgaaatttctccctattttgaatctcgaaataaatttctaaatatatttatgatttattcatcacatatattgtatcttagaatgtctttttaaacatatttaaactttaatcgtcacctgtatatcgatttttgaacctatctctcaaaattttatttataataatgattcatgcacacgtcttctgcgaatgtaggaatggaatacaacactggcgatgatataaatattataataaacacgtaatatgatgaagtagtttctcaacagtgatacaaaacataccgttcatatttcaataaactaatattttaattatatttaatttattttttaattaaaaatattattatattattataaagatttgtcaaatatatttttttttgttaaaaaagtcttttcctttattgtaaaagaaacatttagaattttggaaaaaaagactcaaactaacgttgacatgtgtacagtttaaaataaaatattctattctattaaatataaataaaaaaaagtctcaaataaatagtcaattattacatattattaattataaaataaaatttgctaaacgagaattttttacaaacaattagaattacacataaataaaaatattaataacctgataacttgtaaacatcaagatatgttggaaagcatatttctaatcatctccctaatccaacaaagcattaaagttgtctttaatatattattattattttcaataagctatcttttacccttatttaattggtgaatccgtcacctctaagtttcataatttgttgaaagagttcaaacatggataactacaagacatttcctcattagaaaaatgataaagtttttcaatcattcattcgttatcccccataaaaaaaatccaacatgacagctttgcaataattcttaatcagtaaaataggagccagtgggggcgcccgcatgaaactgagaacgctccacttgcaatggttcgggcccaaattgtgaggtggggcccaatttgatgacaccgttgtaacagattcgacccagcgtctgactccgtcaacattacgaaaatccaaccaccacgcaaattgcaatcctccttttcctagaaggtctccccgaaaacgcatccgacaccatatatcacccgtgtcataagcaccaagtaacgacacaatgacacgcgtcacaatgtgattggagaggattccagcgctttagctataaaatggcccagccccctctgtctctcttcacagttcaattcaaaacaaacttgttcatcctctttgggccgttcctacctctcccttcaaggttcgtccatttcttcgtgtttatgttatttattcgttctgtgaatcatcgttttctcgattaggtcgttgtattttagatagggtttacacggtttaatcgatcttgaattcatcgacagaacattttctttatgttttacaataaggtctggaattgttgattgtgtaatcttatatttggtctagttatttttttttgaagaaattgttcagatccgttaacagagcttttttctttcaataaaatgttcagatctgttaacaaccgccttatgttgtttattttttacgctagattacgttttttccatgaaattctccagatctggcttgagatttacttgatttaattggatagctgttaaattacaacgttgtgattcgtctctgctttttaactctgcag

SEQ ID NO: 30 - DNA, 1521 bp, Phaseolus vulgaris
  source 1..1521: mol_type = genomic DNA
cttgcatcaattgtatcaagaggcttatagttaaaaaaatttagaagtaatgcataaaaaaaaatgcttccacccaattttttaacgacaaatttgctattaattaagtaacaatatcttttttttcatgcgcaatttaaacgtagatatattgtttcacaaaatataaaattggtaataaaaaatttaaaaattaagtatgtttttcatactctggagaaattgattttcgtttttattttaaatttagatctttctaaatatttataataacaaaaaataaatttataataaaaatttgtttattaatataaatatccaacaaatctaaaatttggaggaagaaatataaagattagttattataacattaaagagtaatttacaaaattattaaaaattgaattatttttcagattaaaaaagattattgtaatgatacaatttaattttaaaagagacagtgatagaaattaaattaaggtaatatatatatatatatatatagatatatatatatatatatataggctgcacacaggtaattgcattgactactgatacgatgaagttcatctaatttataaattattcttattatataacaaactatttaatttatttctaaactaatataataatgttaagttcttcgaactataagttagatttcaagctacaatagttgaattttggattggtattcaaataatagactcatcaatttcttaaatagcctaactacgaatgaaaaataaataaaaagagaaagggtgattataagttgttttataacatttttactgaatatttaatataatataaatttataatataatttaaactctcagttattttaataactattacataattactctaattaaaaataaataaaacatattttttctttgaaatttcaacgaagatactaatttaagagggaagcattcgtccttaacgaagaagcaaaatatagaatttgccagagcagtgtagaaagaacgtaatccattgcgtatggaatgcttatcgtgatgtgaacaccgccatacacgtggcaagtctttattcgttagtttatggatatttttgtcattgaatcatcgcatccaagctataaataccacccagaattctcttccttcatacatcgtttattgcaatctcaatctctcagccctaattccccaatctcgcagtttcttgatcattcattttttcccctttcaaggtatgccttctctccctttctcatcttaggttcagttttttgttttagcttatctcatcgataataacgtattcgtaccgatctttctgtttcttttgattgtgttgatagagctgtcttttggattgctatcgattttttttatgtgttggaatatatatcctgatttttagttggaagttgtatagtttcttttacaccttaattttgaggaattgaggtttgtggtttgattgaagtgcctatttatgtttctgtttttgatgatttagtttgtgattgtacag

SEQ ID NO: 31 - DNA, 1990 bp, Phaseolus vulgaris
  source 1..1990: mol_type = genomic DNA
ctctagtcgttgtaggatctctaatacatcccctcacgccaagaccctccaactcatgtgtaagattttaaattaatgggtggtctgatagcaacccggatgctgtgataagtctaacaaactctcgttatgatagactttatatgactctaatatcatcttaagaagtaaactttaagcctaattcattcttataaaatcgacctataagatgagatttggacccacttatatacaatgaaatgtcttaatctttaattgttgtgagatctccaaccattagagaatgtattactcagataacaatatattctatatattatatattaagtgaggaggaatctaagtgtaatttgtgttatataaatattataataaacatctaatatgatgaagtagtttctcaacaagtttaactgtaaatttattataaagtattaaacatacaatacaaaactcatatcacagtacactaatattttaattatatttaatttattttttaatttaaaatattattatattattataatggtttttcaaacatatatatatatattactttttgagtattaaaaaatcttttcctttattataaaaggaacatttagaatttcggaaaaaaataggctaaaatacgcgttgatatgtgtacagtttaaaataaagtattatattatattacatataagtaaaaaagtttctaataaacagccaatttttgtttcttttgttttctttatgacgtaattattacaaactattaattatcaaataaaatgctaaacgataatttttgtacaaacaattaaaattacacataaataaaaaaattaataacctgacaactcagataagatggtgtagcttattatatggaaactagattagatgataatgattatacccatcactcttaaagttgtctttaatacatcattattatttttttcaataatctatcttttactcttatttaattggtgaatccgtcacctctaagtttcataatttgtttagagttaaaacatggataactacaagacatttcctcgttagaaaactgaatgataaagtttttcaatcattcatcgttatcgcccataaaaaaatccaacatgacagcttttaaatagaattcttaatgagtaaaataggagccagtgggcccacatgaaactgtgaacgctccacttgcaatggttcgggcccaaattgtgaagtggggcccaatttgatgacaccgttgtaacagattcgaccgagcgtctgactccgtcaacattacgaaaatccaacaaccacgcaaattgccatcctccttttcctagaaggtctccccgaaaacgcatccaacaccacatatcacccgtgtcataagcaccaagtaacgacacatggacacgcgtcacagtgtcattggacaggattccaccgctttagctataaaatggcccatgccccctgtctctcttcacagttcaattcaaaacaaacttgttcatcctctttaggccgttcccaacctctcccttcaaggttcgttcatttcttcgtgtttatgttatttattcgttctgtgaattatcgttttctcgattaggtcgttgtgttttagttagggtttgcacggtttaatcgatctcgaattcatcgtcagaacatgttctttatcttttacaataacgtctggtattgttgattgtgtaatcttatattcggtctagttatttattttttgaagaaattgttcagatcccttaacagagctttttttctttcaagaaattgttcagatccgttaacagagcttttttctttcaagaaattgttcagatctgttaacaaccgccttatgttgtttgtttttttagctagattatgttttttccatgaaactctccagatctgttcttgagatttacttgattcaattggatagctgtttaattacatcgatgtgattcgtctctggtttttatctctgcag

SEQ ID NO: 32 - DNA, 2036 bp, Phaseolus vulgaris
  source 1..2036: mol_type = genomic DNA
tcaacgcaatatggacccagaataaaattacagaaaataaggcgaaattctacaaattctgaaatctaaattatgtttttatttatctaaaactcaaattttaaaccttattaattataactcaatcctctacttaacataatgctctgaaataagattactaacgttcaatgaggcatggaatttgtctattgccattgtaaaaacaacggagacaaagagtgagattatatatataactataatttgtttagcccaccatatacgttaaagggacaataaaaataatatattttaaaaaatattacaaaattaccaatgaatactccatttgattatatgtttaaattacttcataactcttcactatttggcaatttaattctattctaaacaacttgcatgatatccatcatatactttgctaaagcaacttgttaaaacaaagttaattaataaaaaaaacttgttaaaagatggttgtgtatccaaaagggatgcatccctatattgtttataaagcgttgcatataatatatacacactgtggaacagaaaaaaaataaaaaatcttcagtaagtaagatttacactttcattctaatttctaaaattgcaatatttttaatgtatagttgatgcaattttttaccttttatatttcaatactcatctttttcccttttacgattcatcaacacgctttataaattatttttaatgacaaaaaaattaatgttttataaaagatatattttcaaacaaaaagttattaaaatattatcattaactatttttttaattattattataataaaataaaatatatctataacataatgtcttttcaatatttttaatagtttaagtcgtcatatacaggtgcattaacatgattttttttatcagaaatggtaagataatcagttagaaaaacatatgttaggtggaataaaagtagggatgaaagaacttccggtccatatttcctgatgataataaaaaattgttttcatgtgatgcaaaaatacaaaaatgtacctaatagaaaacatactatgagtttgcttcaaggttcatggtgaactttaaaaaaaaaaacatattatagaaagtgttattgtactagtttcatgttgattaggagttgaaattaatgacagtttaaatacatttttctttcttaacttatcgtcttttaaaaataaaattatagtcttttaaaaataaaattataatgaagcaccgaactctaaagaagtcatccctctacgaatgaaatgataataatggaagcaacatttaaagtggaggaggtttgaaaataccatttaaaataaatgtaaaaagttattgcaatctaaataatcgaataatttttcagagattttaaaaatgaaaactaatttttatttccttccattttcatttcatctaaagaaaacatagtaataatgtattggttgaaaagaaaagaatagtgaagaagtgtcttggtatattgcattgcctccgtgaccggaccgccaatgcaaccaatggatcattcattctagaagctgcctggttccgctcttccttccacgtgtcgcaatgtgataggcaaagaggtaacgccctctttgtcatcgtacctaactcgatcatgtttatccttatataagacacccccaacccctttcccttccatccccaattctatctcattttcattcattcaaacccttcaaggtatccttctcttcaatctttcaatttccaaactttttctcttctatctcttcttcaatctgttagcttttatgcatctgaaaaatctgttaggttgtggacattcatgacattccaggcttccaatttcaattttcaaatgttttgattaagattctcagttttggtgcttctgtgatgttttcttttgattttttatctggagtttagccctaaatttggtcgattattgtaattaggattcttatgtgtattggtttgatttcggttttgattcatgtgttgggttgtttatggcggtgcag

SEQ ID NO: 33 - DNA, 2322 bp, Vigna unguiculata
  source 1..2322: mol_type = genomic DNA
gatcgttgcagtagcagcacaaaactcttaacctatcttttagctaggtgtgaaatcaacattctagtacaattaacttattatttttctataataataataatattattataattataattgttttaataaatatgtgtataataaaaaataaattttgtaattatataaatacaaaatttggaaattaacttagaagattatttttatttaaaattttatttttacatttacttaatctgtattttatttatgaataaatatctattaatattacattttttaattcaacattaaattaaataatcacctctttaaatatttatttatctaacaaattgaattatcacatttgtatttttcaatcaattacttcttagaataattacttctctaaataattattttttaaattataatatattttagataattattttttaaataattagtttcatcaattatttttttttaatatatttatttttctaaatattatttcctcagtacatttatatttctgaaattttatttcttgaaatatattttttttttacaattttatttttcaaataacttttttttttaattttacaattctttacctttcatttttaaataagcaaaattaaaatctattttgataattagattttcctattttttttttttgaaaaattacattaatcatttagaacttaattattgagaaactaaatcctgaatttctataataaactgtattatttgaataattattttgcaaagtatgatgttctcgtaggaaagaaaagtgtaagggtatatgtattgttttagaaacaaatttttgtatgaaagtttttctagaatggatattgtttactttattttattcttgatgtcaaagtcctctacatccagttataataataatatttacgagcctttttctttaaacagaggaaaaaaaagtgaaaagaggggtgaaacaaaaaatgttcataccaataaaaaataatagatggcctttgaaaattatctaaaatgtcaaattgataattatgtctattatgaattaaaaaaaaaaggagtaaaaaaaatctctcctttctaattcaattcagcatttaccttaaaaagtatagcacggaatacaagtaatttaatttcttttcttgcaagataacttttccaaagccgagctcaggcaagggatccatctctaaacttgcaaaatggtgaacccagagagaaaataaatgaattatttagtcaatatttatgatgtaatatataaaatatcaatttagataactgtaattgagagaaaatgagatatataaaaacaaatcgtgatatgtttgacaaaaaatattaactgatttttaatttatttttagattaaattcaatataccttttctaaaatcggaggcggtgatagccaacagattttggaagttgttggaaatctcattagttgccgaaatagattcatgccaaacgttgccgtgtcaacacgggttgcttcaattcgcggtctcaattcttccacgtgtcacaatgtgattgaacaagataccgttgcgtgacgaaacgaatgcttcattttgttataaaaagccaagcccacaccacgcttgaattatcctattcccatcccaacacaactctcgttcgaattgtagcagaagaagaaaaccctagctcaccgtttcaaggtaccttttccaactcttctattttcgattaaatttcggattgtagggtttttattctttcttataaacgattgccctttgattttgatctggttttttgttctttgaatcttttctttattattactttgatttgtttcaaattattggttcgttgaagcgatcgtgaggtgattttatgaaattacatatttaatttattagatctgcgataagtattctgtactgtgtaatcctgagaagtctcttgtaatttagatttgttcagaggtgttttctcttccgtttgggatatctatttgattgatttattgattgaaatacagttatctgggtctcgtgttttcattgctttaccttcttttttgtgctggttttagttcgtgtatgttgtagaccggtccatgtgttttttttcttactagggtttattgattccttaagagggtttcttgaactactttttcagttttcatatatgttagacgtgtctaatttcacgtttcgatcttttgctttgtgctgtctttttactctctcgtgtttactcattgtaattgttgtggttaagaaattggtggctgattattttgttatcttgatcttattgtatcgtctag

SEQ ID NO: 34 - DNA, 2088 bp, Vigna unguiculata
  source 1..2088: mol_type = genomic DNA
catctcgtgttaagcccttcttcattgagttgagaaactgtaagaatttgggccttggcccattttctttgcttttcctttctttcttttcttcttctttatacgtattcctttcttaattattctcgtatgttctcaccctttcccctccatccttttctattaatttatgtcttcaatcatctttttgaaaaatattcgatcatatatatatatatatatatatatatatatatatatatatatatatatatatatatatatatatataatatgtaattatattgatcaatgaattatgtattatgttttgaaaaaaaaaaactggaaaaactttttacatcttaaataatattatatgtgttttgtttcccaaaaaccctttttgttagcgcatacaaaacatcttttatgtattctacaaaatagcatatcatatttttaaaaaataggtattttatttattcatcgcgcatattctatcttagaatgatttcttaaacatatttaaactttaatactcacccatatatcaatttttaaaaacaactctcaaaattttttgttttttaatttatacgaatattatattgcatacctaatataacaaataagtttctcaacaagtttaattgatgataaaaggaacattaacaccatctagatgagagggaaaaaatcagtttgaaacacgcgttgatatgtctatattagtaaaataaaaatattatattaaagcataatacatataaataatacatataagtaaaaaaacactcaaatataacagtcaatttttgtttttttttctcattttatgatatttttcttttataaagtacatacaattaattattaaataaaacgcaaaacgagaatattagtagtataaacaatagagctgcacttatatataaaaatattaataatacaattatgcttcttaaagccatcaacaactgtataatctgacaacttgtccactgcagatcagatggtgtagcttattcatattagatgataaggatgcccatcgctcttaagttttctttactatatcatttttgttttttcaattaagctgtcctatacccttattttattggtaaatccgtcacctttaagtttcatgatttgtttaaatagttaaagcgtggatgattacaagacatttccttattaaaaaaataaatgataaagctttccaatcattcattagtccattaaaaaatcaaacatcacagctttgcaatagatttcttaatcatgtaaaataggagccaaccgttggtgtagtggatagaggggcccatacaaaactgagaacgctccacttgcagtggttccggcccaaattgtgaagtggggtccaatttaataacgccgttgtaacagattaagctgacgtaggcgcgtctgactccgtcaacattaccaacccgctaaccaccccgcaaattgcaatcctgaatttccaagaaaggactccgaaaacgcatcagacaccacatatcacccgtgtaataagcaccaaatagggacacaaggacacgcgtcacaatgtgattggagaggattccacccctttagctataaaaaggcccacaccctctgtctctcttcacagttcaattcaaaacaaactacttcattctctttgcgcagttccctacctctcccctcaaggttcgtctattttattttgtctatgttattcattagtcccgtgaatgatcatgttttcgattagcttgttgagtttaagatagggtttgtacagtttcatcgatcgtcagaatcttttctttctcttttacaataacacgtcttgaatttttctctgtagttctttattacgttaattgttttctttttaagaaaattttcagatccgttaacagtcttcttattttaaagaaaaaaaaaattcagatccattaacaaccgccttgtttggttaatcctgaccgaagattatgtatttttttccctgaaattaatcagatctgttcttgagatgtacttaattcagtcggatagctgttaaatttcatcattctgattcgtttgtggttttcaactttgcag

SEQ ID NO: 35 - DNA, 2263 bp, Vigna unguiculata
  source 1..2263: mol_type = genomic DNA
ggcccaacgacccggataggttgtgctccacgacttgtacagattgggcaagcccgatgccaatgacgggctcgacgacttagtcgtgttaggtcgggccaggacgacgacgacttgatcgtgtcgggccagcatgacgagttagacagaccagatcgacccgacgacctaaacaagtgacttggcctagcttgaagacttggacaatgtaaagtttaataatatatttttcatgtttaagaagaaagtccatggacgggccttgacccacaggatttttgtatgatttttggtcgtgtgggcttctttaatgggttttttttcatgtggatcaaggcctgcgtgggccagaacatagccttgtggattgggccaaattgacaattctaaccaaattaaattgtgtcggtcttaaagacacatatgttttaaaaaatttaatttaaatttattttataaaatagattctgaattttgaatttgatctaaatatgatcttaaaaaaattcaaattaattttacttaaaaataaatttgaataaaaactctaatctaattatttaaatttaaaatgagtggagatcggatcattaaaaaccaatcctattgatataagcataggttgtgccgaggccacaaataaagtatacccgaaaagagaagcagaggagaaaagaagtaaccatgtgcaacagtacgaggattttagcatacttcaccagtagaaatttaagggaatgtaacatatgttaatgtcgtatattcatattgtgtaagcatttgtaacaacagtggaataaaacatcatgctggtgtaaaaggacagggtaatagattgcagagacttagcccacttgccattgcaacttctcctacggcctaaactaggaaaaaatattatttaataccaataattagtgtttggaaatgtaaaatgattccgacaaaataaatttttatatttttgtaaagaaaaaaataaagaaataaagataatatcaaataaatattaaaaaataaaaaattaaatgttgttttgatttttttaattttcaataaaaactaaaattaatccatccttaaaatgttagtccaaattaattcttaatttaaaaaaaatgtgtgatgtttgatacatcaaaataatttaacaaaatttaattaaaaaattaaatttatatatttctaaaattaaaaagctaaattgatttaaaattttgaaaaaaattcattaaaagataaaagaacaaaaacatattcatccaaaaataaaaaaattgtaatgtgaaaatatcattgtctctaataatatatctttttaaaacaaataatttttacaaatattttttatttttattttttcaattcgtgattatttaaaaagattaaaacagactcttggtttagtaaaaggtattagaatttccggtttataatttaaaatctgataaaatttaaataaatagttttttaggtcaaatttaaataacatgacttttataaacttcagaaaatagatataaaatcttaaaaagcagtatgacgtaataaaaataaaaataaataaattaaaatggtatcctaacaaactaagtcaataatacattctttgaaggaaaaatatccaaaaaccagaccgccaatgcaaccaatggattttatatttcaacgtcatgattctagaagctacgctcttccttacacacgtcgcagtgtcattggccaagaggtaacgctctctttgttttcgtacccaacttcatcaccgcctcatcatgtttttcctatataacacaccccaacccccttatcatttccatccccaattcaatctcatatttttcgtttctgttcaatccaatccttcaaggtatgtttctcttctccacctgttttttcaattcaatattgtctctggaaaatctgttaggtttttaattttacggtaattggtgtatggttctgcgaacatgaagtgtatcaatgtcgttgtaggcttttcgctgctgcctttaaaacctgataatgaaaatcttgtggacgttcttgaaatttcaggcttcgatttttcagttttgaatgtttcaattattctgattaagattctcagttttgatttctttgtttggggaccatgatttttatctggagttgaaccctaaatttggtcaattattctaattaagattcttatgtgtttgtgcgtgtttatttggtttgatttcgattttgattcatctgttggttggatatgacggtgcag

SEQ ID NO: 36 - DNA, 1306 bp, Arabidopsis thaliana
  source 1..1306: mol_type = genomic DNA
atcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 37 - DNA, 912 bp, Glycine max
  source 1..912: mol_type = genomic DNA
caatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacag

SEQ ID NO: 38 - DNA, 2262 bp, Glycine max
  source 1..2262: mol_type = genomic DNA
cttggaattatgggattggataaacgtttactaaacatggaaagcgtttattaaaaaaattaagcatttattattccatttttttatagaaattattccatattttattcatggtgtaattaatattcttttcccataacaacttttaattactcaaacttatctttaggggacattttggtagaagaaaagttgtttccatcctaattatattgcaaggtggctgcattcatgattttaagaatatatttttgtagttcactgtaaaattattgaaaatatttacaattttttttcggaattaaagattttcattttcaataaaatactaagtatatgagtatacttttgtactacattatttttcttttactattaattacttttgatcttttccattaatttacaggtttattgaattttcgtatgccaaccttttaaattttattttatagtcatttttctaaattttgcaaacaaactatagcaataaaaaaatagactagtttttaataaattacaaataaacttatttttgctacacatttttttataacatatttctttacacgtgtgctgttgttggaataaagaaaaaaaaatgcattaaccaaatactatgaagtagtttgattttagatcacttgactaagactaaaatacatgcgcagctaacaccaaaagtctacggggtttaggttgaatccaaactgaaagtttgagaaaatcggtatattaattgtcatacttcctccagacttaaataatataagttaaaaaaaatagacctaaataaaggtaaatattaactgagtattttatcttatttaataataatattcctattatgttgtttatgtaacctgtttctaattaattcattaataaaataaaaaaactaaaaagagactaattcatttaaattaataaaattaaacagggactaataaaaaaaaactaattaaacatgaactaatttattaaaattaataaaaactaattaaacaggaactaattcattaaaattaaaaaaataaagagagactaattcatttaaattaataaaattaaagagagactaattaaaattaataaaactaattaaaaagggactaattcattaaaattaacaaaacttattaaacatgggcctaaaattaaaaaaataaaatgagacttattcatttaaatcatttttttatgaactcttgtaattttacattttacatgtgtttttaaaaattcacataatccttttaaatcttaaaaatctataaaggtctttcaattcttatgatataaatctattaaaatttaaattatcataaaaattttataaaaaaaaattaataaatcattacattcttatttaatctttaaaattttaaaaaaaattatgctaaaataatttttttaaatcttaattcaatacatcttctaaaatttttgtttttacccgataaaataataagtataacaaataattgtagcaaaaccagatgataaaagataaagaaaaaaggaaaaaaaaagggaaattttaaattacatatttactctaactttcatttttataaactattaatgaattttataaatataatttaatattgtgtgagccaaattgttattaggataacaatagagaggttgatctcaatagtagtcttttatcatcaaaatataccttgaggtgtaattataattactttattaataaattttataagtatttataagtatttttactatacatttttcacatgattttttcatattatatataatatattaattttgtactatatatttaacggttagaaacattaaatttattaataatttatattttttaaaattatctttaaaattaacatgtgctagtactttttgcaatcttcatacaagaaaatgaatttgtcttattaaattttaccatttattattaaataactaaaggtattttgataagaaaaaatcattagggatggaggtaatatttattaggaaaacattgtaaaacactaaaacccatcaggatatcgaccgaaggaacattttattactaaaccttaaaaagaaatattgcaataatcttcaaaattaagccattttaatttttattcttattgaatgataaaaaaaattaataattaaataacatttctcaaacactcttaaaccctagttgtttcctctgtctcaaaacaaaaaccctacaaacaaaacacttcctgcttcgcaaacgtttgaatctgaatc

SEQ ID NO: 39 - DNA, 484 bp, Arabidopsis thaliana
  source 1..484: mol_type = genomic DNA
aaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagcc

SEQ ID NO: 40 - DNA, 2066 bp, Glycine max
  source 1..2066: mol_type = genomic DNA
ggtatcagaaaccaaaggagacaacatctagatcagcaggtggaggatctaaacagctaggtgaatgagatccatcagaatatttgtacaagcctaaggatcttgtcttgccgtccctcatttaaagaagagtgttacatagatatctaacatactctctgtatcatttgtgtgatatatcatacttcaattatttttgcaaatcaattatgccattgttggtttagaattatatattttttgagtcactagaactagcgtcacaactgatgtgttaaattctctaatggttttgccaatgctgcaaggctaaaaatccgggttgaataatttaaggtaacttatgcaactaccactttagatatataataaaataaaattgcctaacttgtttttaaaaataaagatttttgtaacattaatgataaaaaaaaattaatttgtatgtcatgtttgatattcatgatttaacttagaattttgtaaaatctaatataaaattatgagatgagactcggaatcttccataatttatgacatcttacaaaactaagataaaatcaaactaataattttttttttccaatggacagctaaaatagaattggaacagtaaatatgtttgttaggctgtctgcacgcaaaagccaattcctgccgtaaatctctgcagtgaggcattctgttggacagtgttctgcggcttggagctaaaattttcgattccctctactttaaaagcattaccacgtcaatgcacgtgcatgtcaaggtcaaagagtaaattggttccaattgaagaatatcttgtgtccagctctctgttgatacagcaaaatgaatttcatctatcttccgtgagtaaacaagttcaatttgtgcttaaatttgttttatgaagagtgcaacctgaagcattgaaaacaatgaaaggcatcttgaaataatgaaatcatatatcttagatgtagacctccatccccgttaaaaagaaagtacactattttgggtgatggactcaatgactttaaagagagaaatttacgtacgctgacttttccttattttttttataagtaaacaacaacttaaataatagtttatataagtgaatatcaacttaaacaaatcgctcgcaactcactttccttctttaaagtcattgaatccttgccaattattttgtgagtatttgtaaatttaactttgaattattttgattttataaaattaattttagtaaaaagtgagttgaaattgaaatcatttatgtttatatttttttctcaatttcgtattaaagtttaagataaaacttttaacagagtaagagttatatttattttcatttttattgaaatatatgtttttaggagtaattttaattttggttaaaaatcaacaaacaactgttttttttttcttttgcaaaatctcattacacacggtgtcgtagaaccaaacatataattactaatacggatattactcattttagaattatgctgataattaatcgattaattataactttgctctcatttatatttttttcatttctctttatattgccctcttcattttacatcaaagtatccaaaaagtgtgactttgattaaattatatataccaaccttttttttataaaaaaaacctttcaaattgaaagactacaaatcagtgtggatttttacgtgacagataatattataatttcggtaaaattataatttggtcatttacttttttttatttagtcatctgttttatttattaattttgataacatatttcttatttttatttcaatttgatcttatttgtcacaaatttatttaacacaaaatacacacggacagataaagcaaatgaaaatgcaataaaagaaaatagcgggatcgctaaccgtttgctttaacgagcatcatcttattctcgtttcccgctctatatatattgattccctcacttcttcagacctcatttataattcaaaaacctctctctctctctctctctctctctcaattcttcaggcaccttcaaaatttctctctagagtcacg

SEQ ID NO: 41 - DNA, 1420 bp, Glycine max
  source 1..1420: mol_type = genomic DNA
cctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatc

SEQ ID NO: 42 - DNA, 770 bp, unidentified
  source 1..770: mol_type = unassigned DNA
cgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 43 - DNA, 1061 bp, Arabidopsis thaliana
  source 1..1061: mol_type = genomic DNA
ttgcaaacagaggagatatactaagcctttggaagagtgtatagatgagctagaactctacacacttggtcaatactagatttatctgcatcaaatgctttccaccagggtggattctctgggagaggtacttgaaacctacgtgcagcagcatacacgaccccacaagccagaacctcacttctgaaccttacacaaagggttgtacgcagactatcattagccaaattccaagcttcttgcctcaattccggaggtgtttctagtgttcaacatgacaaacaaaacccatctctttcagtatatgtctctcagttgtgcttaattcaaatttcaactcagagaacttcttggcatacttatccagattatctaatgatctcatctaatggtaattcaactttcagtataagtctcgcagcaaactatctttacatcaaatttttaacaactcaatgcacaaaatacttttcctcaacctaaaaataaggcaattagccaaaaacaactttgcgtgtgaacaacgcgtacacgtccctacacatacctgtcaatttataattggctattgcttccacgccttagctttctcgtgaccgaccgagtcgtcctcgtcttttttgcttctataaatcaaatacccaaagagctcttcttcttcacaattcagattccaattttctcaaactctaaaatcaatctctcaaatctctcaaccgtgatcaaggtagatttctgagttcttattgtatttcttcgatttgtttcgttcgatcgcaatttaggctctgttctttgattttgatctcgttaatctctgatcggaggcaaattacatagtttcatcgttagatctcttcttatttctcgattagggttcgtatttttcgcagatctgtttattttcttgttgtttccttgtatttgatccgatttgttgaaagaatttgtgtgttctcgattatttacgctttgatctgtgatttttatctagatttggtgttagtttcttgtttgtgcgatcgaatttgtcgattaatctcggtttatctgattaacag

SEQ ID NO: 44 - DNA, 387 bp, Arabidopsis thaliana
  source 1..387: mol_type = genomic DNA
ctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 45 - DNA, 456 bp, unidentified
  source 1..456: mol_type = unassigned DNA
gttttggtctgaggaataatctgttttacatgaacatcaattagttaagatgttgttttgtccagaaaattgtggtgattctaaccaacactcacatcacatgtaaattatgaaatggaatacaattaaaatgaactttgtaggagtcttttgcaccctcaaatgtaattcctcaaaagcctatatatgtcgtcctctaccatagcattagatatatattggtaggagttaggacagtcttaaaaggagagctcaaacgcttaaacataatgaagccttgaaacctacagcaataaaataagcatataatataacatttaattcaaaagcttaagccagcaatgaaagtgaacataatggaaggggaagaagttttagtttacatttataagtcccacatcgtgtatttcataaaagaaactagtctttataatcttatgacaaggctatcagctt

SEQ ID NO: 46 - DNA, 438 bp, Pisum sativum
  source 1..438: mol_type = genomic DNA
ctagacatgattctcgcaaggatactgttttttcgcattacagaaattcaaggaagaagctgaaattattcgttttctcacgggatggaacgagtgcaatcttttcctgatatgatgaacaaacttttctttagactttttcaagatccgcgggtgattgttaaggcaactgtagctaatcgatctgttccatcttttgttttaagtgcatctttacaaacctcgccgttttaatgatcaaaatctaccatgagtttgtaggaagctatacacaactggtagattgaattcatttgagattgctgtggttgattgaagaattctaagccagtagtgtattattgaagaaaaacgtgtgatagatattttccaagtcccacattgtttataatatagcatttgtaatagtttatatactttggatacacaacagtgtta

SEQ ID NO: 47 - DNA, 471 bp, Pisum sativum
  source 1..471: mol_type = genomic DNA
gaggtatccgcaacatcgcattgaccgtattttcctgtcatataattgtttgcagcataactgcaatgatttaatagcatgcttgtatgcaaactatatcaaacaaatggtgaatatggaagaacaatgtacaacatatttttggacataatcaaaatgtttgagatatggtaaaaaaatcatatgcaaaactgtcttaaggaaaaaagaattggcagagcgtcgggaaaacgatttgagagaagacttgctactatagaaataagactccccgacttaaaatcgatttctttttgtcttgttttccaatatgcaatagagactaagtgcaatttcttctctagtattattattattatttcacatatgttccaacataatactacaccattttagatccatttaagtcccacattggctagaatatcacacaagcaagtgcttataaatgtctgacatgctagttagttc

SEQ ID NO: 48 - DNA, 473 bp, Pisum sativum
  source 1..473: mol_type = genomic DNA
gaggtatccgcaacatcgcattgaccgtattttcctgtcatataattgtttgcagcataactgcaatgatttaatagcatgcttgtatgcaaactatatcaaacaaatggtgaatatggaagaacaatgtacaacatatttttggacataatcaaaatgtttgagatatggtaaaaaaatcatatgcaaaactgtcttaaggaaaaagaattggcagagcgtcgggaaaacgatttgagagaaggcttgctactatagaaataagactccccgacttaaaatcgatttctttttgtcttgttttccaatatgcaatagagactaagtgcaatttcttctctagtattattattattattatttcacatatgttccaacataatactacaccattttagatccatttaagtcccacattggctagaatatcacacaagcaagtgcttataaatgtctgacatgctagttagttc

SEQ ID NO: 49 - DNA, 421 bp, Pisum sativum
  source 1..421: mol_type = genomic DNA
ccaaagatatactatacttatacttagatttatagttagatgcaactttaaaaaagtatttttcgtgataaaaaaataataataatactgctcccatactattatatgcatttgatttcaaagcaaataaatacaataaatggcatctactatatagatttaaactccatagtcaattcaaagatggaagcaaaacacttaaaaaatacatcttatatttgcctaacaacaaatagatttacaagatactcttcctcttcattaagttaaaaaaaaatgaagttttgaagtgattataagtgtatttcgtttgtaataattaaaattactcttttggttgagtttctcgttgataagtcccacatcggcaagcttatcaaagaaataacactttataatagtctgacaaagtagatagctc

SEQ ID NO: 50 - DNA, 468 bp, Pisum sativum
  source 1..468: mol_type = genomic DNA
gaggtatccgcaacatcgcattgaccgtattttcctgtcatataattgtctgcagcataactgcaatgatttaatagcatgcttgtatgcaaactatatcaaacaaatggtgaatatggaagaacaatgtacaacatatttttggacataatcaaaatgtttgagatatgttaaaaatttcatatgcaaaactgtcttaaggaaaaaagaattggcagagcgtcgggaaaacgatttgagagaaggcttgctactatagaaataagactccccgacttaaaatcgatttctttttgtcttgttttccaatatgcaatagagactaagtgcaatttcttctctagtattattattatttcacatatgttccaacataatactacaccattttggatccatttaagtcccacattgcctagaatatcacacaggcaagtgcttataaatgtctgacatgctagttagttc

SEQ ID NO: 51 - DNA, 467 bp, Pisum sativum
  source 1..467: mol_type = genomic DNA
gaggtatccgcaacatcgcattgatcgtattttcctgtcatataattgtttgcagcataactgcaatgatttaatagcatgcttgtatgcaaactatatcaaacaaatggtgaatatggaagaaaaatgtacaacatatttttggacataatcaaaatgtttgagatatggtaaaaaatcatatgcaaaactgtcttaaggaaataagaattggcagagcgtcgggaaaacgatttgagagaaggcttgctactatagaaataagactccccgacttaaaatcgatttctttttgtcttgttttccaatatgcaatagagaccaagtgcaatttcttctctagtattattattatttcacatatgttccaacataatactacaccattttggatccatttaagtcccacattgcctagaatatcacacaagcaagtgcttataaatgtctgacatgctagttagttc

SEQ ID NO: 52 - DNA, 492 bp, Pisum sativum
  source 1..492: mol_type = genomic DNA
cgtatggatctaaagtctagcaacatcttgcttgatgaagattatgagaaaattgctgaatttggtgttgcaaggtttgcttaaaaatagcttttttgctggcacccatgattatattgctccaggtatgtttcgataatgctttcaacatgaaattttaatatactgttttttaatttcaaaagtatctcatgctttatttacacaattactgtttgtgacattttatcataagcattatggaactacaaatgcagcataaaagtattttgaggtatccgcaacatcgcattgaccgacttaaaatcgatttctttttgtcttgttttccaatacgcaatagagaccaagtgcaatttcttctctagtattattattatttcacatatgttccaacataatactaaaccattttggatccatttaagtcccacattgcttagaatatcacacaagcaagtgcttataaatatctgacatgctagttagttc

SEQ ID NO: 53 - DNA, 494 bp, Pisum sativum
  source 1..494: mol_type = genomic DNA
gaggtatccgcaacatcgcattggccgtaatttcctgccatataaaagtttgcagcataaccgcaactgcaaccacgatttaaagtatgctagtaagccaactataacaaacaaacagtgaatatggaagatgccgatgggatgctaaatattaggaagcattagagaaaagccagaaaatgaagcagcaacactcaaaagttataatgttgattgttgatactctaagctaaaccaggtatgcatttcactacattggttttgaatcatttttttgagtgtatttagtttgcgtataactttctgaatagatttaatccttctatatatgttgtgactaattcagtcctatatatgtattataacatcaaccacaacatttaagaaagcatgttctaacttaatactacaccattctagattcattaagtcccgtgttggctagaacatcacacaagtaagtgtttataaatgtctgatatgctagttagttc

SEQ ID NO: 54 - DNA, 493 bp, Pisum sativum
  source 1..493: mol_type = genomic DNA
gattaagaaatatggatgtttatttaaatgatagaggaatttgaattgtatttctttaattttatatatacattttgttagcatataattaaaattgaagagtgtagagttgaaatgaatttgagtaaattaaatcatgcaaactccatagtataattagactttagccaattaaaaagaaaatagagatagtttataaataaatataaaaaagaaagaaagaaagagatagaaatgaggtgagaagccagtgaaaagagtaacaaaggtgtacaaaatgcaggagctagctactgcagtcggtgctcttataagcatcatctatgtgaaaagagtgaggaaggaacaaaactttcattttgtgtatagtgattgtgatggcaatgaatcgcatcttacgtgatattgttgcttggttgtctgagaagtcccacaccggttacgtttgcaagttatttattatttatataatacacacatatcatctgttatc

SEQ ID NO: 55 - DNA, 480 bp, Pisum sativum
  source 1..480: mol_type = genomic DNA
ggatgtttatttaaatgatagaggaatttgaattgtatttctttaatttttatatatacatttttgttagcatataattaaaattgaagagtgtagagttgaaatgaatttgagtaaattaaatcatgcaaactccatagtataattagactttagccaattaaaaaagaaaatagagatagtttataaataaatataaaaaaagaaagaaagaaaagagatagaaatgaggtgagaagccagtgaaaagaaaggtgtacaaaatgcaggagctagctactgcagtcggtgctcttataagcatcatctatgtgaaaagagtgaggaaggaacaaaactttcatttttgtgtatagtgattgtgatggcaatgaatcgcatcttacgtgatattgttgcttggttgtctgagaagtcccacaccggttacgtttgcaagttatttattatttatataatacacacatatcatctgttatc

SEQ ID NO: 56 - DNA, 485 bp, Pisum sativum
  source 1..485: mol_type = genomic DNA
ggatgtttatttaaatgatagaggaatttgaattgtatttctttaattttatatatacattttgttagcatataattaaaattgaagagtgtagagttgaaatgaatttgagtaaattaaatcatgcaaactccatagtataattagactttagccaattaaaaaagaaaatagagatagtttataaataaatataaaaaaagaaagaaagaaaagagatagaaatgaggtgagaagccagtgaaaagagtaacaaaaggtgtacaaaatgcaggagctagctactgcagtcggtgctcttataagcatcatctatgtgaaaagagtgaggaaggaacaaaactttcatttttgtgtatagtgattgtgatggcaatgaatcgcatcttacgtgatattgttgcttggttgtctgagaagtcccacaccggttacgtttgcaagttatttattatttatataatacacacatatcatctgttatc

SEQ ID NO: 57 - DNA, 498 bp, Pisum sativum
  source 1..498: mol_type = genomic DNA
gattaagaaatatggatgtttatttaaatgatagaggaatttgaattgtatttctttaatttttatatatacatttttgttagcatataattaaaattgaagagtgtagagttgaaatgaatttgagtaaattaaatcatgcaaactccatagtataattagactttagccaattaaaaaagaaaatagagatagtttataaataaatataaaaaaagaaagaaagaaaagagatagaaatgaggtgagaagccagtgaaaagagtaacaaaaggtgtacaaaatgcaggagctagctactgcagtcggtgctcttataagcatcatctatgtgaaaagagtgaggaaggaacaaaactttcatttttgtgtatagtgattgtgatggcaatgaatcgcatcttacgtgatattgttgcttggttgtctgagaagtcccacaccggttacgtttgcaagttatttattatttatataatacacatatcatctgttatc

SEQ ID NO: 58 - DNA, 490 bp, Pisum sativum
  source 1..490: mol_type = genomic DNA
gattaagaaatatggatgtttatttaaatgataggaatttgaattgtatttctttaatttttatatatacattttgttagcatataattaaaattgaagagtgtagagttgaaatgaatttgagtaaattaaatcatgcaaactccatagtataattagactttagccaattaaaaagaaaatagagatagtttataaataaatataaaaagaaagaaagaaagagatagaaatgaggtgagaagccagtgaaaagagtaacaaaggtgtacaaaatgcaggagctagctactgcagtcggtgctcttataagcatcatctatgtgaaaagagtgaggaaggaacaaaactttcatttttgtatagtgattgtgatggcaatgaatcgcatcttacgtgatattgttgcttggttgtctgagaagtcccacaccggttacgtttgcaagttatttattatttatataatacacacatatcatctgttatc

SEQ ID NO: 59 - DNA, 545 bp, unidentified
  source 1..545: mol_type = unassigned DNA
gcttgcgtgacatactagggagacaggaaccttgcttgaacaggatcggtttctagggagacaggaacctaagttcatcacctatagtacaaaactatcaacaaaataggttacataatgtttgcagattctttttatttcattgcattgcttagaatgcactttctcttgaaccattaagctttgaaatgagataaaacttattgttagagctagaaaaacaagcttgaaatttctattttatactctagttttccaagtttacagaagctaacactaatagcaccattttaccacacactttggagtcagttctaaaatattttaagaatttcagaaacagatatagtatagcaacattgagtatagaaaaattcatcaaactttatactaaattgatgagcattgatagtaacaaggcgtgacaacaaaagacagaaacctcattttttaaactaatactacaaacatgaaatatagtcccacatcttttggttcaggattatgctaaatgtttatatacagcacgcggagagctttggtca

SEQ ID NO: 60 - DNA, 548 bp, Pisum sativum
  source 1..548: mol_type = genomic DNA
gacaatggcttcacattaatctagcgataattgaatttcaacacaaatatagcttgtgaataagtactcaccttgcaacagagaagctgtattgaacacaagtttcagcagcatgcagaattatatctcccatgcttttgtcctggaagttgttaacattgcaaatgaatagagcaaatagcaatcatatataaatcaaaccaacaatggtctataaagttatgcaatcatcacaggactcacatatttgcataaacatacaatatttacacctaattttataataataagaatgctgctcagaaatgatctacaaaaccccaaatgaataataaaattgaaattccaagatacaccagagaaacttcaatataattacacaaacataaacaatatataaagatcaaatcatcattatgcaccactcttgtgacattttaacataaataaaactagagttacagcaaaaactattatattactcccacaccgttgaagcccagaaaatcttaaacttttatatatctcagtctaaccgctgatgaaca

SEQ ID NO: 61 - DNA, 546 bp, Pisum sativum
  source 1..546: mol_type = genomic DNA
ggcttcacattaatctagcgataattgaatttcaacacaaatatagcttgtgaataagtactcaccttgcaacagagaagctgtattgaacacaagtttcagcagcatgcagaattatatctcccatgcttttgtcctggaagttgttaacattgcaaatgaatagagcaaatagcaatcatatataaatcaaaccaacaatggtctataaagttatgcaatcatcacaggactcacatatttgcataaacatacaatatttacacctaattttataataataagaatgctgctcagaaatgatctacaaaaccccaaatgaataataaaattgaaattccaagatacaccagagaaacttcaatataattacacaaacataaacaatatacaataaacatcaaatcatcattatgcaccactcttgtgacatttttaacataaataaaactagagttacagcaaaaactattatattactcccacaccgttgaagcccagaaaatcttaaacttttatatatctcagtctaaccgctgatgaaca

SEQ ID NO: 62 - DNA, 560 bp, Pisum sativum
  source 1..560: mol_type = genomic DNA
cagcacaaagacaatggcttcacattaatctagcgataattgaatttcaacacaaatatagcttgtgaataagtactcaccttgcaacagagaagctgtattgaacacaagtttcagcagcatgcagaattatatctcccatgcttttgtcctggaagttgttaacattgcaaatgaatagagcaaatagcaatcatatataaatcaaaccaacaatggtctataaagttatgcaatcatcacaggactcacatatttgcataaacatacaatatttacacctaattttataataataagaatgctgctcagaaatgatctacaaaaccccaaatgaataataaaattgaaattccaagatacaccagagaaacttcaatataattacacaaacataaacaatatacaataaacatcaaatcatcattatgcaccactcttgtgacattttaacataaataaaactagagttacagcaaaaactattatattactcccacaccgttgaagcccagaaaatcttaaacttttatatatctcagtctaaccgctgatgaaca

SEQ ID NO: 63 - DNA, 542 bp, Pisum sativum
  source 1..542: mol_type = genomic DNA
cagcccaaagacaatgacttcacactaatctagcgataattgaatttcaacacaaatatagcttgtgaataagtactcaccttgcaacagagaagctgtattgaacacaagtttcagcagcatgcttttgtcctggaagttgttaacatggcaaatgaatagagcaaatagcaatcatctataaatcaaaccaacaatggtctataaagttatgcaatcatcacaagactaacatatttgcataaacatacaatatttacacctaattttataataagaagaatgctgctcagaaatgatctacaaaaccccaaatgaataataaaattgaaattccaagatacaccagagaaacttcaatataattacacaaacataaacaatatacaataaacatcaaatcatcattatgcaccactcttgtgacatttttaacataaataaaactagaggtacagcaaaaactattatattcctcccacatcggtgaaacccagaaaatcttaaacttttatatatctcagtctaactgctgatgaaca

SEQ ID NO: 64 - DNA, 549 bp, Pisum sativum
  source 1..549: mol_type = genomic DNA
cgcatcagctattcacagctcaaatgaaagtaaagggataatgataattttgtattcatagattggaattgaaatcagtgtatcaaactgcttgtaaacaaaatacactaactaagagtatagaaccatgaggtgacacgtattatcagttaatttaacgctgccagtctatccatccaaagcagaattcttctgaggagagaaaatttgtttgattttaatgaaaatttccatatgacaaattaaaattgattaaataaatgaattagctactgataataactaccactaaatataaataaaaagaaaagaactttagaaacatattaacgatttcatctataaaatcttaccaatcatgaaaagatgcccctgtttgttgaaatggtaacaaaatctggaaagattaagagacttatcagaagcattttttttgaaatataagtcccaaactgagagctacaacgaaaggcattatattactcccacaacggttaagtgaagaaaatcataaacgtttatataaccaaatcagagcacttaaaagca

SEQ ID NO: 65 - DNA, 532 bp, Pisum sativum
  source 1..532: mol_type = genomic DNA
gtgactcggtacagaaacatgctatatatacaccttattttataataataagatagaacccaaccaaaattaaatttcctatgctgtttaccatctatgtctttgaactccaagtactgcctatggcgtgttcaaattgcttgtaagtaacatacacttaaatcagcaatgtttaccgtatatccacctaaagcggaactgagtcctgtggcgaaaaggattctataaaaagcaagcaatttaatctacataggacaattttaaattgcttaaagaaatgaagtatccactattgctgctaactactgataaatataactgcgttttagaaacatatttacagtcacccatacaatcttatcagtgttttgttttgttacaaaatctgcaaagattatgagacttaacaggaacatttttttcataaataaataaatcccaaacgcagcagaaaccattacattactcccacatctttcaaccgcaacaaatcttcactgtttatataccttaggctaaccacagttgtgca

SEQ ID NO: 66 - DNA, 513 bp, Pisum sativum
  source 1..513: mol_type = genomic DNA
aggctgctgtgtaccagtggtggtgatgggtgaatttgttgcgagtttcttctcctctaaaacagaagaaactttcactacggaaaatcactcactcgcataaagaaagaacctgcataaagaaagagcaaatgtttatctatgtttgtttagaatgacagaattggttgaactttgaactacatgatatataaatcaagagttatacagaaaatgaaggatattggaattgcagagattaaagcatgaataacttgaataactattccaaccgttctcctatggatgtatgttgttaagaaatgagaggggttacaatttgacagaaaggggttattgagtataaaaggaacttgcggtaagtaatacatacttagttatagtaaagcttgaatccaaatctgaaagcaagactcacattagaaaagaaaaaaaatgaagactttagtcccacaccgctaaagctcatagaaaatctacggtttatataagcgagaagaagaccaaacgtca

SEQ ID NO: 67 - DNA, 526 bp, Pisum sativum
  source 1..526: mol_type = genomic DNA
gcagcttccttccttaaaaggctgctgtgtaccagtggtggtgatgggtgaatttgttgtgagtttcctcctctaaaacagaagaaactttcactacggaaaatcactcactcgcataaagaaagaacctgcataaagaaagagcaaatgtttatctatgtttgtttagaatgacagaattggttgaactttgaactacatgatatataaatcaagagttatacagaaaatgaaggatattggaattgcagagattaaagcatgaataacttgaataactattccaaccgttctcctatggatgtatgttgttaagaaatgagaggggttacaatttgacagaaaggggttattgagtataaaaggaacttgcggtaagtaatacatacttagttataaagcttgaatccaaatctgaaagcaaaactcacattataaaagaaaaaaaatgaagactttagtcccacaccgctaaagctcatagaaaatctactgtttatataagcgagaagaagagcacaagtca

SEQ ID NO: 68 - DNA, 534 bp, Pisum sativum
  source 1..534: mol_type = genomic DNA
cagttaagcagcttccttccttaaaaggctgctgtgtaccagtggtggtgatgggtgaatttgttgtgagtttcctctcctctaaaacagaagaaactttcactacggaaaatcactcactcgcataaagaaagaacctgcataaagaaagagcaaatgtttatctatgtttgtttagaatgacagaattggttgaactttgaactacatgatatataaatcaagagttatacagaaaatgaaggatattggaattgcagagattaaagcatgaataacttgaataactattccaaccgttctcctatggatgtatgttgttaagaaatgagaggggttacaatttgacagaaaggggttattgagtataaaaggaacttgcggtaagtaatacatacttagttataaagcttgaatccaaatctgaaagcaaaactcacattataaaagaaaaaaatgaagactttagtcccacaccgctaaagctcatagaaaatctactgtttatataagcgagaagaagagcacaagtca

SEQ ID NO: 69 - DNA, 536 bp, Pisum sativum
  source 1..536: mol_type = genomic DNA
ggacgttcaacggaaacaacctaaagtttttgatgcctaggcaatcttaattgcagctgcatcaggtgtactaaagatctttgcatgtttcaaacaacatgtgatgccttaataaatcatggtaattccaccccaagaggaaggtagtgtatgcatcactacaatgtaatgcagacatgttaagaaaacttaaagttgttcatttgttaaataaacagtaacatttgaagcaattaaactctgctgttgagttgaggtcagtatttaactcaaataaacatatgctttctcatgtttttacaaccaagatttaaaaaataaccaacgacgaatcttaattgctgtatcgttaatggttgatgtttaaaatcttgaataagcttcataatgagtttagacaaattcatgaaacgttttgccaaacttgtgagtagaatatagtatagtaggcgtttcagaaattgaaagagtcccacatcgtttgctttaggcatttaaaaaatgtttatataacgataagacgagaccaatagaca

SEQ ID NO: 70 - DNA, 536 bp, Pisum sativum
  source 1..536: mol_type = genomic DNA
ggacgttcaacggaaacaacctaaagtttttgatgcctaggcaatcttaattgcagctgcatcaggtgtactaaagatctttgcatgtttcaaacaacatgtgatgccttaataaatcatggtaattccaccccaagaggaaggtagtgtatgcatcactacaatgtaatgcagacatgttaagaaaacttaaagttgttcatttgttaaataaacagtaacatttgaagcaattaaactctgctgttgagttgaggtcagtatttaactcaaataaacatatgctttctcatgtttttacaaccaagatttaaaaaataaccaacgacgaatcttaattgctgtatcgttaatggttgatgtttaaaatcttgaataagcttcataatgagtttcgacaaattcatgaaacgttttgccaaacttgtgagtagaatatagtatagtaggcgtttcagaaattgaaagagtcccacatcgtttgctttaggcatttaaaaaatgtttatataacgataagacgagaccaatagaca

SEQ ID NO: 71 - DNA, 455 bp, Pisum sativum
  source 1..455: mol_type = genomic DNA
gataattccgagtgttagaaaaatctataaatgaagagtataaaattgattatttaaagaaaaattgattaataagatataattaattgaataatgagtaaagaataatatggttaatggatagtagtaatgaattattgagtaattagtgaatgagtaattatatataggaataattaataaagatgatagtaatgcattagttagtactattttttacagcacatataatactagttgaagaattttgttgactatggtgtgtagaattaatgatgatacacatataattaattactactaattaagtgaagttgaggcaacataacaattactattaattactactaagcggaaggagaagtaagtaagtgtgttatgtttgtttagttccacatcgactatttagtttcattttattatgtttatataatacagctactcacccactgttt

SEQ ID NO: 72 - DNA, 570 bp, Pisum sativum
  source 1..570: mol_type = genomic DNA
cgtgcgacgcgctcaagtatacatgaagagtatacaaattaaaaattgagtggataatcatatacaaattaaaaataaaaggagaattttaaaaatcaattaactaaaaaaattagataattcactaagtgtttgaatgaaattgattatttaaagaaaaattgattaataagatataattaattgattaatgagtaaagaataacatggttagtgaatagtactaatgaattattgagtaaagagtaattaataattatttaaagaataattctatatacgaataattaataaagatgatagtaatgcattaattagtttttttttacagcacatagaatactagttgaagagttttgttgaccctggtttgtagaattaatgatgatacaaatataattaattactaggactaattaagtgaagttgaggcaacagaacaattattaactactactaagcggaaggagaagtaactgtgttatgtttgtttacttacttttagtaccacatcgactgtttagtttcatattgttatgtttatataatagagttactcacaccttgatc

SEQ ID NO: 73 - DNA, 393 bp, unidentified
  source 1..393: mol_type = unassigned DNA
atggaaaacaaatgtatttggattgctcgcttgcaatccgaggaatatatccttggaatattttaatctgctcttgcatgggctctatatgatgcattgatatccttcaacttatatggcttagtgtgaaaaaaatagtgtaactagggaagggtatagacagattgtgaatgaattggagaagctaaagatggaacgagcagatgaagttaagtaagtgatatacttgcggtggagcaatgcatggttaaggcaggatatgatgaggcaatatagtcatccagaaaaagcatcggcgggcaagactgagcgtataccaattactggagtcccacatcgcttaaatgttatgcatcttcagtgtttatatatcgtcacagagcacaattgctt

SEQ ID NO: 74 - DNA, 300 bp, Glycine max
  source 1..300: mol_type = genomic DNA
aaatggtaaaatgtcaaatcaaaactaggctgcagtatgcagagcagagtcatgatgatactacttactacaccgattcttgtgtgcagaaaaatatgttaaaataattgaatctttctctagccaaatttgacaacaatgtacaccgttcatattgagagacgatgcttcttgtttgctttcggtggaagctgcatatactcaacattactccttcagcgagttttccaactgagtcccacattgcccagacctaacacggtattcttgtttataatgaaatgtgccaccacatggatt

SEQ ID NO: 75 - DNA, 304 bp, Glycine max
  source 1..304: mol_type = genomic DNA
aaacaagctaaaagttgcaaaagaaatggcagggctataaggctcacctactcctggatttaccaaattttggttcgtccctatactcgaaaaataaaacaaaataaatttcagtatcttcgtttttgtatgctttgactgtgaggcgaggccaactttcttcttctgtctgagatgaattttgtttgcctcctgtgaaggatgtatcattcaaagtgaatgttttgcaactgccagtagtcccacatcgaccaaatattcttattacagtgtgtttatatagcacctggagaaggaatgggtt

SEQ ID NO: 76 - DNA, 325 bp, Arabidopsis thaliana
  source 1..325: mol_type = genomic DNA
tttactttaaattttttcttatgcagcctgtgatggataactgaatcaaacaaatggcgtctgggtttaagaagatctgttttggctatgttggacgaaacaagtgaacttttaggatcaacttcagtttatatatggagcttatatcgagcaataagataagtgggctttttatgtaatttaatgggctatcgtccatagattcactaatacccatgcccagtacccatgtatgcgtttcatataagctcctaatttctcccacatcgctcaaatctaaacaaatcttgttgtatatataacactgagggagcaacattggtca

SEQ ID NO: 77 - DNA, 439 bp, Glycine max
  source 1..439: mol_type = genomic DNA
ggcatgtgactttttatttaaattattctcagaaatattaaaatataatcaaatatatttttttataagatactggaaataacatttttaggaatgtaacgaaagccccattacaaacacttgaactcagggatcgatccaacttaattattatctgctacttcaaattcaaatttataggccctacctttatgttttgctctgcactttcttaaatggaaacaagtaacacaatagagtaagatcatttacttgtaagcggaaactgttgcatcaactgcaatattttcgggttagttttataatataacaatgaagagaattgccgagaagatcatatcaagtcccatattccatttattggcctttacaagtcccacatcggtccaaatattagacaaagaaacatttatatatcgtctgacaaacctgtaagatt

SEQ ID NO: 78 - protein, 1264 aa, Moraxella caprae
MLFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHRNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNR

SEQ ID NO: 79 - DNA, 2604 bp, synthetic construct
  source 1..2604: mol_type = other DNA
gttgctgaatcagaagttgagtgaggtttggaggcaactgttgggacaacggcttagacaaacattttgatgctacaatttatccaatttttagagggttttcatttacgccctatatatttttcagaaattttatggaagttcaaaagttttcttgttgatgcgtgtgacacgtggcagcatttgatctaatatgagtgtgttcgaaaattatctttaggagattattatgaaataaaatagaaagagtttgtttgaaagtaaactttaggacactgaaaatagaatctctgaaaaatatataggataaaatgtaacgagtaattctacatttttataaattttggaaccactcaattttgtcaacgagtttcagtcttctgctagagtttcggaaattattaccgctttcttcttgtcactgttctgtatttatataacatcatctgtgagagtttcgaaaattgttacagctttcttctgaccactgttctgtatttctataacagattaaatgaatctaaaaatcaaaatctacttataaaagagaatagagggtagtgacacgtggcagatttaatgaacccaaatgaattgtcttctatatatattagtttatttttagtcatatccaactttatgttgcaatcttaatagtggactaaaaaagatctaaaaatattaattcacaagctaatcaaagcatgactactatacctaatgttgttttaagaaaaaaatttcacaacataggtaaacatataactaaatttttatattgagttttactgtatacttataaaatatttgacaattttttttcaaagatacattgattagctgttctcaaatatccaatttcaaatacataagacaatacttttcaaacacaaattttatttgatgtcgggcttatcattttgagaaattgtctttttatagaagttgagcacacttaattttattgttcatttctttattataacttcacgatgtgtatgcaacattagggcagtttgttttaactttaagaaaataattttcttttaaatggattttacaaaaatgttttttaaaatattataagtttttctaaatttatttttgataaattaaaaatttgaattattcattctataatataaatatacattattaaagatcaaaacatagtcaaaatcacaattttttcaaaaaaattatatcttaaaaataatttttatgaaaagctatttgaaatagcttcaaaattaagtgattttttaaaattttgatatctaaattttttttcgataaaatgatgaaatacctaaaataacattttaagaataattattcaaaccaaatttttatttgaagtttttatgaaaagtttttttataaactttatttagattaaaatatgtaacactgtaacaatcatttaaaaaaaaaccaaaacaaatggatcctaaattaattaaatttttttttgagatttttctgatacattccaatttatattatcggtgctcatcccaaattcactatatcatttgtattcttttaaaaattaattttggcattttttagttatacgcactttccacgaaataagccaacagatgtgacacgtggcaggatggcaaagctatcatcttatccgttgccgaagacgactgtgataccaaacattgccgtgtcaaaatcgtttatagctcagattgtgacacgtggcaggatgtgattggaagagctgccgttacttttacgcaacctcgaatcattttaagctataaattcgaaagcccaatttcacagctttcatatattcatatactttcgtttcgttgcagagaagagaaaccctagcacactgaatcaaggtatcttctacccttcatctttttctcaagaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacacgtaattcttcagatccgtttgttatgattaaattttcagatctgattatgtatcaattgtatccttgggaaattttctttgattaattggcaaatttcgttcaatatttttatgatcgtgacacgtggcagtctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 80 - DNA, 2000 bp, Solanum lycopersicon
  source 1..2000: mol_type = genomic DNA
atttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcag

SEQ ID NO: 81 - DNA, 2056 bp, synthetic construct
  source 1..2056: mol_type = other DNA
atttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacacgtaattcttcagatccgtttgttatgattaaattttcagatctgattatgtatcaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcag

(19) United States
(12) Patent Application Publication
(10) Pub. No.: US 2025/0011801 A1
(43) Pub. Date: Jan. 9, 2025

(54) PROMOTER ELEMENTS FOR IMPROVED POLYNUCLEOTIDE EXPRESSION IN PLANTS

(71) Applicant: Benson Hill, Inc., St. Louis, MO (US)

(72) Inventor: Ross A. Johnson, Olivette, MO (US)

(73) Assignee: Benson Hill, Inc., St. Louis, MO (US)

(21) Appl. No.: 18/708,497

(22) PCT Filed: Nov. 9, 2022
(86) PCT No.: PCT/IB2022/060794

§ 371 Date: May 8, 2024

Related U.S. Application Data
(60) Provisional application No. 63/277,370, filed on Nov. 9, 2021.

(51) Int. Cl. C12N 15/82; C12N 9/22; C12N 15/11

(52) U.S. Cl. CPC C12N 15/8216; C12N 9/22; C12N 15/11; C12N 15/8213; C12N 2310/20

(57) ABSTRACT

Provided herein are compositions and methods for expressing a polynucleotide of interest in a plant or plant part. Compositions include nucleic acid molecules comprising a promoter sequence, and DNA constructs comprising the promoter molecule operably linked to one or more polynucleotides of interest. Plants and plant parts comprising the compositions or regenerated according to the methods are also provided.

### CROSS-REFERENCE TO RELATED APPLICATIONS

This application claims the benefit of U.S. Provisional Application No. 63/277,370 filed on Nov. 9, 2021, the entirety of which is hereby incorporated by reference.

### FIELD OF THE INVENTION

The present disclosure relates to compositions and methods for expressing a polynucleotide of interest in a plant or plant part.

### REFERENCE TO AN ELECTRONIC SEQUENCE LISTING

The contents of the electronic sequence listing (BH00109PCT_SL_v1-2002-11-03.xml, Size: 176,727 bytes; and Date of Creation: Nov. 8, 2022) is herein incorporated by reference in its entirety.

### BACKGROUND OF THE INVENTION

Expressing a polynucleotide of interest in a plant or plant part is useful for product development. For instance, expression of editing reagents, e.g., a nuclease and a guide RNA (gRNA), is a major determinant of the frequency of heritable mutations introduced in plant genomes and technical viability in crops. The processes following expression of editing reagents include the localization and complexation of the nuclease with the guide RNA, search for the plant genomic target, and catalysis of a double-strand DNA break.

Expression of a polynucleotide of interest may be enhanced by optimization of upstream regulatory elements to drive expression of the polynucleotide of interest. In the gene/genome editing context, gains in the efficiency of introducing heritable edits have been described to stem from the optimization of upstream regulatory elements to drive expression of the editing reagents early in the primary-transformant generation. Such optimization in the editing context can enable a given insertion-deletion modification product concept with fewer explant inputs for transformation, lesser downstream edit event screening, and simplified segregation. Gains in the efficiency of heritable edits are also likely to enable edits based on sequence complementarity, such as homology-directed repair. Accordingly, optimization of upstream regulatory elements for a polynucleotide of interest, such as a nuclease and a guide RNA, may be useful in enhancing expression of the polynucleotide of interest, and could have important commercial advantages.

### SUMMARY OF THE INVENTION

Compositions and methods for regulating gene expression in a plant or plant part are provided. Compositions can include nucleic acid molecules comprising a promoter molecule for expressing a polynucleotide of interest, or DNA constructs comprising the promoters operably linked to polynucleotides of interest. The promoters can enable high expression levels and/or favorable expression patterns of one or more polynucleotides of interest, particularly editing reagents, e.g., nucleases and guide RNAs. Methods of expressing a polynucleotide of interest in a plant or plant part by introducing the compositions of the present disclosure are also described. Plants and plant parts comprising the compositions or being regenerated according to the methods of the present disclosure are also described.

In some aspects, the present disclosure provides nucleic acid molecules comprising a promoter sequence, wherein the promoter sequence has transcription initiation function and comprises one or more synthetic motif sequences, wherein each synthetic motif sequence comprises: (i) a nucleic acid sequence that has at least 95% sequence identity to any one of SEQ ID NOs: 1-3; or (ii) the nucleic acid sequence of any one of SEQ ID NOs: 1-3. In some embodiments, the promoter sequence comprises one or more linkers, wherein one of the linkers connects the one or more synthetic motif sequences and the promoter sequence, and/or the one or more synthetic motif sequences are at least two synthetic motif sequences, and one of the linkers connects two of the at least two synthetic motif sequences. In some embodiments, the promoter sequence of the present disclosure comprises two synthetic motif sequences, wherein each synthetic motif sequence comprises: (i) a nucleic acid sequence that has at least 95% sequence identity to any one of SEQ ID NOs: 1-3; or (ii) the nucleic acid sequence of any one of SEQ ID NOs: 1-3. In some embodiments, said two synthetic motif sequences are different synthetic motif sequences.

In some embodiments, at least one of said synthetic motif sequences comprise a nucleic acid sequence that has at least 95% sequence identity to SEQ ID NO: 1, or the nucleic acid sequence of SEQ ID NO: 1, and at least one of said synthetic motif sequences comprise a nucleic acid sequence that has at least 95% sequence identity to SEQ ID NO: 2, or the nucleic acid sequence of SEQ ID NO: 2.

In some embodiments, said one or more synthetic motif sequences are inserted into the 5′ untranslated region (UTR) of said promoter sequence. In some embodiments, said one or more synthetic motif sequences are inserted into a 5′ intron of said promoter sequence.

In some embodiments, the promoter sequence of the nucleic acid molecule comprises: (i) a nucleic acid sequence that has at least 80% sequence identity to SEQ ID NO: 6, further comprising the one or more synthetic motif sequences inserted therein; or (ii) the nucleic acid sequence of SEQ ID NO: 6, further comprising the one or more synthetic motif sequences inserted therein. In some embodiments, the promoter sequence of the nucleic acid molecule comprises: (i) a nucleic acid sequence that has at least 80% sequence identity to any one of SEQ ID NOs: 9-11 and 79-81 or (ii) the nucleic acid sequence of any one of SEQ ID NOs: 9-11 and 79-81.

In some embodiments, the promoter sequence of the nucleic acid molecule comprises at least one mutation cluster at positions selected the following position clusters when aligned for maximum full-length (i.e., global) identity with the PsUBI3-SYN3p promoter: a) at least one of 167, 169, 170, 171, 176, 177, 178, 179, and 180; b) at least one of 561, 562, 563, 570, 571, 572, 573, 574; c) at least one of 1446, 1607, 1608, 1609, 1611, 1617, 1618, 1619; d) at least one of 1706, 1707, 1708, 1709, 1716, 1718, 1719, 1894; and e) at least one of 2229, 2230, 2231, 2232, 2237, 2238, 2239, 2240, 2241. In specific embodiments, the promoter sequence of the nucleic acid molecule comprises a mutation at each position of each mutation cluster set forth in the following positions when aligned for maximum full-length (i.e., global) identity with the PsUBI3-SYN3p promoter: a) 167, 169, 170, 171, 176, 177, 178, 179, and 180; b) at least one of 561, 562, 563, 570, 571, 572, 573, 574; c) 1446, 1607, 1608, 1609, 1611, 1617, 1618, 1619; d) 1706, 1707, 1708, 1709, 1716, 1718, 1719, 1894; and e) 2229, 2230, 2231, 2232, 2237, 2238, 2239, 2240, 2241. In specific embodiments, the promoter sequence of the nucleic acid molecule comprises at least one mutation cluster selected from the following mutation clusters when aligned for maximum full-length (i.e., global) identity with the PsUBI3-SYN3p promoter: a) at least one of C167G, A169G, T170A, T171C, T176G, C177G, T178C, T179A, T180G; b) at least one of T561G, A562T, T563G, T570G, T571G, A572C, T573A, T574G; c) at least one of C1446G, T1607G, C1608T, A1609G, G1611C, T1617G, T1618C, T1619A; d) at least one of T1706G, G1707T, T1708G, C1709A, T1716G, G1718A, C1719G, C1894A; and e) at least one of A2229T, A2230G, G2231A, G2232C, C2237G, C2238G, A2239C, T2240A, T2241G. In particular embodiments, the promoter sequence of the nucleic acid molecule comprises each of the following mutation clusters when aligned for maximum full-length (i.e., global) identity with the PsUBI3-SYN3p promoter: a) C167G, A169G, T170A, T171C, T176G, C177G, T178C, T179A, T180G; b) T561G, A562T, T563G, T570G, T571G, A572C, T573A, T574G; c) C1446G, T1607G, C1608T, A1609G, G1611C, T1617G, T1618C, T1619A; d) T1706G, G1707T, T1708G, C1709A, T1716G, G1718A, C1719G, C1894A; and e) A2229T, A2230G, G2231A, G2232C, C2237G, C2238G, A2239C, T2240A, T2241G.

In some aspects, the present disclosure provides DNA constructs comprising, in operable linkage: (a) a nucleic acid molecule comprising a promoter sequence/molecule and (b) a polynucleotide of interest. In some embodiments, the promoter sequence/molecule of the DNA constructs has transcription initiation function and comprises, in operable linkage: (i) one or more synthetic motif sequences, wherein each synthetic motif sequence comprises a nucleic acid sequence that has at least 95% sequence identity to any one of SEQ ID NOs: 1-3 or the nucleic acid sequence of any one of SEQ ID NOs: 1-3, optionally with any additional features disclosed herein; or (ii) a nucleic acid sequence that has at least 80% sequence identity to any one of SEQ ID NOs: 6-8 and 12-35, and retains transcription initiation function, or a nucleic acid sequence of any one of SEQ ID NOs: 6-8 and 12-35.

In some embodiments, the polynucleotide of interest of the DNA construct of the present disclosure encodes a guide RNA (gRNA) and/or a nuclease.

In some embodiments, the polynucleotide of interest of the DNA construct encodes a guide RNA, and the DNA construct further comprises, in operable linkage: (a) a promoter molecule comprising one or more synthetic motif sequences, said synthetic motif sequence each comprising a nucleic acid sequence that has at least 95% sequence identity to any one of SEQ ID NOs: 1-3 or the nucleic acid sequence of any one of SEQ ID NOs: 1-3; or a promoter molecule comprising a nucleic acid sequence that has at least 80% sequence identity to any one of SEQ ID NOs: 6-43 and 79-81, and retains transcription initiation function, or a nucleic acid sequence of any one of SEQ ID NOs: 6-43 and 79-81; and (b) a polynucleotide of interest encoding a nuclease.

In some embodiments, the polynucleotide of interest of the DNA construct encodes a nuclease, and the DNA construct further comprises, in operable linkage: (a) a promoter molecule comprising one or more synthetic motif sequences, said synthetic motif sequence each comprising a nucleic acid sequence that has at least 95% sequence identity to any one of SEQ ID NOs: 1-3 or the nucleic acid sequence of any one of SEQ ID NOs: 1-3; or a promoter molecule comprising a nucleic acid sequence that has at least 80% sequence identity to any one of SEQ ID NOs: 6-35, 43-77, and 79-81, and retains transcription initiation function, or a nucleic acid sequence of any one of SEQ ID NOs: 6-35, 43-77, and 79-81; and (b) a polynucleotide of interest encoding a guide RNA.

In some embodiments, the nuclease encoded by the polynucleotide of interest of the DNA construct is a clustered, regularly interspaced, short palindromic repeats (CRISPR)-associated Cas endonuclease. In some embodiments, the CRISPR nuclease is a Cas12a nuclease. In some embodiments, the Cas12a nuclease is McCpf1. In some embodiments, the nuclease is further operably linked to one or more nuclear localization sequences (NLSs) and/or one or more epitope tags.

In some embodiments, the DNA construct of the present disclosure further comprises, in operable linkage, a nucleic acid molecule encoding a selectable marker and/or a regulatory RNA. In some embodiments, the DNA construct further comprises a promoter molecule operably linked to the regulatory RNA and comprising: (i) a nucleic acid sequence that has at least 80% identity to any one of SEQ ID NOs: 6-35, 43-77, and 79-81, and retains transcription initiation function; or (ii) a nucleic acid sequence of any one of SEQ ID NOs: 6-35, 43-77, and 79-81.

In some aspects, the present disclosure provides cells comprising the nucleic acid molecule (e.g., comprising a promoter sequence) or the DNA construct of the present disclosure. In some embodiments, the cell is selected from the group consisting of a plant cell, a bacterial cell, and a fungal cell.

In some aspects, the present disclosure provides plants or plant parts comprising the nucleic acid molecule (e.g., comprising a promoter sequence) or the DNA construct of the present disclosure.

In some aspects, the present disclosure provides plants or plant parts comprising, in operable linkage: (a) a promoter molecule having transcription initiation function and comprising one or more synthetic motif sequences, wherein each synthetic motif sequences comprises (i) a nucleic acid sequence that has at least 95% sequence identity to any one of SEQ ID NOs: 1-3 or (ii) the nucleic acid sequence of any one of SEQ ID NOs: 1-3; and (b) a polynucleotide of interest.

In some embodiments, the plant or plant part comprises a promoter molecule that comprises one or more linkers, wherein: one of the linkers connects the one or more synthetic motif sequences with a promoter sequence within the promoter molecule; and/or the one or more synthetic motif sequences are at least two synthetic motif sequences, and one of the linkers connects two of the at least two synthetic motif sequences. In some embodiments, the promoter molecule of the plant or plant part comprises two synthetic motif sequences, said synthetic motif sequences each comprising a nucleic acid sequence that has at least 95% sequence identity to any one of SEQ ID NOs: 1-3 or the nucleic acid sequence of any one of SEQ ID NOs: 1-3. In some embodiments, said two synthetic motif sequences are different synthetic motif sequences.

In some embodiments, at least one of said synthetic motif sequences comprise a nucleic acid sequence that has at least 95% sequence identity to SEQ ID NO: 1, or the nucleic acid sequence of SEQ ID NO: 1; and at least one of said synthetic motif sequences comprise a nucleic acid sequence that has at least 95% sequence identity to SEQ ID NO: 2, or the nucleic acid sequence of SEQ ID NO: 2.

In some embodiments, said one or more synthetic motif sequences are inserted into the 5′ UTR of said promoter molecule. In some embodiments, said one or more synthetic motif sequences are inserted into a 5′ intron of said promoter molecule.

In some embodiments, the promoter molecule of the plant or plant part comprises: (i) a nucleic acid sequence that has at least 80% sequence identity to SEQ ID NO: 6, further comprising the one or more motif sequences inserted therein; or (ii) a nucleic acid sequence of SEQ ID NO: 6, further comprising the one or more motif sequences inserted therein. In some embodiments, the promoter molecule of the plant or plant part comprises: (i) a nucleic acid sequence that has at least 80% sequence identity to any one of SEQ ID NOs: 9-11 and 79-81 or (ii) the nucleic acid sequence of any one of SEQ ID NOs: 9-11 and 79-81.

In some embodiments, the promoter sequence of the plant or plant part comprises at least one mutation cluster at positions selected the following position clusters when aligned for maximum full-length (i.e., global) identity with the PsUBI3-SYN3p promoter: a) at least one of 167, 169, 170, 171, 176, 177, 178, 179, and 180; b) at least one of 561, 562, 563, 570, 571, 572, 573, 574; c) at least one of 1446, 1607, 1608, 1609, 1611, 1617, 1618, 1619; d) at least one of 1706, 1707, 1708, 1709, 1716, 1718, 1719, 1894; and e) at least one of 2229, 2230, 2231, 2232, 2237, 2238, 2239, 2240, 2241. In specific embodiments, the promoter sequence of the plant or plant part comprises a mutation at each position of each mutation cluster set forth in the following positions when aligned for maximum full-length (i.e., global) identity with the PsUBI3-SYN3p promoter: a) 167, 169, 170, 171, 176, 177, 178, 179, and 180; b) at least one of 561, 562, 563, 570, 571, 572, 573, 574; c) 1446, 1607, 1608, 1609, 1611, 1617, 1618, 1619; d) 1706, 1707, 1708, 1709, 1716, 1718, 1719, 1894; and e) 2229, 2230, 2231, 2232, 2237, 2238, 2239, 2240, 2241. In specific embodiments, the promoter sequence of the plant or plant part comprises at least one mutation cluster selected from the following mutation clusters when aligned for maximum full-length (i.e., global) identity with the PsUBI3-SYN3p promoter: a) at least one of C167G, A169G, T170A, T171C, T176G, C177G, T178C, T179A, T180G; b) at least one of T561G, A562T, T563G, T570G, T571G, A572C, T573A, T574G; c) at least one of C1446G, T1607G, C1608T, A1609G, G1611C, T1617G, T1618C, T1619A; d) at least one of T1706G, G1707T, T1708G, C1709A, T1716G, G1718A, C1719G, C1894A; and e) at least one of A2229T, A2230G, G2231A, G2232C, C2237G, C2238G, A2239C, T2240A, T2241G. In particular embodiments, the promoter sequence of the plant or plant part comprises each of the following mutation clusters when aligned for maximum full-length (i.e., global) identity with the PsUBI3-SYN3p promoter: a) C167G, A169G, T170A, T171C, T176G, C177G, T178C, T179A, T180G; b) T561G, A562T, T563G, T570G, T571G, A572C, T573A, T574G; c) C1446G, T1607G, C1608T, A1609G, G1611C, T1617G, T1618C, T1619A; d) T1706G, G1707T, T1708G, C1709A, T1716G, G1718A, C1719G, C1894A; and e) A2229T, A2230G, G2231A, G2232C, C2237G, C2238G, A2239C, T2240A, T2241G. In some embodiments, the promoter molecule(s) and/or the polynucleotide(s) of interest are stably inserted in the genome of said plant or plant part.

In some embodiments, said plant is selected from the group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In some specific embodiments, said plant is Pisum sativum.

In some aspects, the present disclosure provides methods of expressing a polynucleotide of interest in a plant or plant part comprising introducing a DNA construct into a plant cell, wherein the DNA construct comprises, in operable linkage: (a) a nucleic acid molecule comprising a promoter molecule and (b) a polynucleotide of interest. In some embodiments, the promoter molecule has transcription initiation function and comprises: (i) one or more synthetic motif sequences, wherein the one or more synthetic motif sequences each comprise a nucleic acid sequence that has at least 95% sequence identify with any one of SEQ ID NOs: 1-3 or the nucleic acid sequence of any one of SEQ ID NOs: 1-3, optionally with any additional features disclosed herein; or (ii) a nucleic acid sequence that has at least 80% sequence identity to any one of SEQ ID NOs: 6-8 and 12-35, and retains transcription initiation function, or a promoter sequence comprising a nucleic acid sequence of any one of SEQ ID NOs: 6-8 and 12-35. In some embodiments, the methods further comprises regenerating a plant or plant part from said plant cell.

In some aspects, the present disclosure provides methods of expressing a polynucleotide of interest in a plant or plant part comprising:
introducing a DNA construct into a plant cell, wherein the DNA construct comprises, in operable linkage: (a) a promoter molecule having transcription initiation function and comprising one or more synthetic motif sequences, wherein the one or more synthetic motif sequences each comprise: (i) a nucleic acid sequence that has at least 95% sequence identify with any one of SEQ ID NOs: 1-3; or (ii) the nucleic acid sequence of any one of SEQ ID NOs: 1-3; and (b) a polynucleotide of interest; and
regenerating a plant or plant part from said plant cell.

In some embodiments, the promoter sequence of the methods of the present disclosure comprises a promoter molecule that comprises one or more linkers, wherein: one of the linkers connects the one or more synthetic motif sequences with a promoter sequence within the promoter molecule; and/or the one or more synthetic motif sequences are at least two synthetic motif sequences, and one of the linkers connects two of the at least two synthetic motif sequences. In some embodiments, the promoter molecule of the methods comprises two synthetic motif sequences, said synthetic motif sequences each comprising a nucleic acid sequence that has at least 95% sequence identity to any one of SEQ ID NOs: 1-3 or the nucleic acid sequence of any one of SEQ ID NOs: 1-3. In some embodiments, said two synthetic motif sequences are different synthetic motif sequences.

In some embodiments, at least one of said synthetic motif sequences comprise a nucleic acid sequence that has at least 95% sequence identity to SEQ ID NO: 1, or the nucleic acid sequence of SEQ ID NO: 1; and at least one of said synthetic motif sequences comprise a nucleic acid sequence that has at least 95% sequence identity to SEQ ID NO: 2, or the nucleic acid sequence of SEQ ID NO: 2.

In some embodiments, said one or more synthetic motif sequences are inserted into the 5′ UTR of said promoter molecule. In some embodiments, said one or more synthetic motif sequences are inserted into a 5′ intron of said promoter molecule.

In some embodiments, the promoter molecule of the methods of the present disclosure comprises: (i) a nucleic acid sequence that has at least 80% sequence identity to SEQ ID NO: 6, further comprising the one or more synthetic motif sequences inserted therein; or (ii) the nucleic acid sequence of SEQ ID NO: 6, further comprising the one or more synthetic motif sequences inserted therein. In some embodiments, the promoter molecule comprises: (i) a nucleic acid sequence that has at least 80% sequence identity to any one of SEQ ID NOs: 9-11 and 79-81 or (ii) the nucleic acid sequence of any one of SEQ ID NOs: 9-11 and 79-81.

In some embodiments, the promoter sequence of the methods disclosed herein comprises at least one mutation cluster at positions selected the following position clusters when aligned for maximum full-length (i.e., global) identity with the PsUBI3-SYN3p promoter: a) at least one of 167, 169, 170, 171, 176, 177, 178, 179, and 180; b) at least one of 561, 562, 563, 570, 571, 572, 573, 574; c) at least one of 1446, 1607, 1608, 1609, 1611, 1617, 1618, 1619; d) at least one of 1706, 1707, 1708, 1709, 1716, 1718, 1719, 1894; and e) at least one of 2229, 2230, 2231, 2232, 2237, 2238, 2239, 2240, 2241. In specific embodiments, the promoter sequence of the methods disclosed herein comprises a mutation at each position of each mutation cluster set forth in the following positions when aligned for maximum full-length (i.e., global) identity with the PsUBI3-SYN3p promoter: a) 167, 169, 170, 171, 176, 177, 178, 179, and 180; b) at least one of 561, 562, 563, 570, 571, 572, 573, 574; c) 1446, 1607, 1608, 1609, 1611, 1617, 1618, 1619; d) 1706, 1707, 1708, 1709, 1716, 1718, 1719, 1894; and e) 2229, 2230, 2231, 2232, 2237, 2238, 2239, 2240, 2241. In specific embodiments, the promoter sequence of the methods disclosed herein comprises at least one mutation cluster selected from the following mutation clusters when aligned for maximum full-length (i.e., global) identity with the PsUBI3-SYN3p promoter: a) at least one of C167G, A169G, T170A, T171C, T176G, C177G, T178C, T179A, T180G; b) at least one of T561G, A562T, T563G, T570G, T571G, A572C, T573A, T574G; c) at least one of C1446G, T1607G, C1608T, A1609G, G1611C, T1617G, T1618C, T1619A; d) at least one of T1706G, G1707T, T1708G, C1709A, T1716G, G1718A, C1719G, C1894A; and e) at least one of A2229T, A2230G, G2231A, G2232C, C2237G, C2238G, A2239C, T2240A, T2241G. In particular embodiments, the promoter sequence of the methods disclosed herein comprises each of the following mutation clusters when aligned for maximum full-length (i.e., global) identity with the PsUBI3-SYN3p promoter: a) C167G, A169G, T170A, T171C, T176G, C177G, T178C, T179A, T180G; b) T561G, A562T, T563G, T570G, T571G, A572C, T573A, T574G; c) C1446G, T1607G, C1608T, A1609G, G1611C, T1617G, T1618C, T1619A; d) T1706G, G1707T, T1708G, C1709A, T1716G, G1718A, C1719G, C1894A; and e) A2229T, A2230G, G2231A, G2232C, C2237G, C2238G, A2239C, T2240A, T2241G. In some embodiments according to the methods of the present disclosure, the polynucleotide of interest encodes a guide RNA (gRNA) and/or a nuclease.

In some embodiments according to the methods of the present disclosure, the polynucleotide of interest encodes a guide RNA, and the DNA construct further comprises, in operable linkage: (a) a promoter molecule comprising one or more synthetic motif sequences, said synthetic motif sequence each comprising a nucleic acid sequence that has at least 95% sequence identity to any one of SEQ ID NOs: 1-3 or the nucleic acid sequence of any one of SEQ ID NOs: 1-3; or a promoter molecule comprising a nucleic acid sequence that has at least 80% sequence identity to any one of SEQ ID NOs: 6-43 and 79-81, and retains transcription initiation function, or a nucleic acid sequence of any one of SEQ ID NOs: 6-43 and 79-81; and (b) a polynucleotide of interest encoding a nuclease.

In some embodiments according to the methods of the present disclosure, the polynucleotide of interest encodes a nuclease, and the DNA construct further comprises, in operable linkage: (a) a promoter molecule comprising one or more synthetic motif sequences, said synthetic motif sequence each comprising a nucleic acid sequence that has at least 95% sequence identity to any one of SEQ ID NOs: 1-3 or the nucleic acid sequence of any one of SEQ ID NOs: 1-3; or a promoter molecule comprising a nucleic acid sequence that has at least 80% sequence identity to any one of SEQ ID NOs: 6-35, 43-77 and 79-81, and retains transcription initiation function, or a nucleic acid sequence of any one of SEQ ID NOs: 6-35, 43-77 and 79-81; and (b) a polynucleotide of interest encoding a guide RNA.

In some embodiments according to the methods of the present disclosure, the nuclease is a clustered, regularly interspaced, short palindromic repeats (CRISPR)-associated Cas endonuclease. In some embodiments, the CRISPR nuclease is a Cas12a nuclease. In some embodiments, the Cas12a nuclease is McCpf1. In some embodiments, the nuclease is further operably linked to one or more nuclear localization sequences (NLSs) and/or one or more epitope tags.

In some embodiments according to the methods of the present disclosure, the polynucleotide of interest is stably inserted into the genome of the plant or plant part.

In some embodiments according to the methods of the present disclosure, expression or function of one or more molecules encoded by the polynucleotide of interest is increased in the plant or plant part relative to a control plant or plant part, wherein in the control plant or plant part comprises the polynucleotide of interest operably linked to a control promoter that does not comprise any one of: (a) a promoter molecule comprising one or more synthetic motif sequences, wherein the one or more synthetic motif sequences each comprise a nucleic acid sequence that has at least 95% sequence identity to any one of SEQ ID NOs: 1-3 or the nucleic acid sequence of any one of SEQ ID NOs: 1-3; or (b) a promoter molecule comprising a nucleic acid sequence that has at least 80% sequence identity to any one of SEQ ID NOs: 6-8 and 12-35, and retains transcription initiation function, or the nucleic acid sequence of any one of SEQ ID NOs: 6-8 and 12-35.

In some embodiments according to the methods of the present disclosure, the one or more molecules encoded by the polynucleotide of interest are a guide RNA and/or a nuclease, and an efficiency of introducing a mutation to a genome of a plant or plant part is increased by about 10% to about 500% in the plant or plant part relative to the control plant or plant part.

In some embodiments according to the methods of the present disclosure, the DNA construct further comprises, in operable linkage, a nucleic acid molecule encoding a selectable marker and/or a regulatory RNA. In some embodiments, the DNA construct further comprises a promoter molecule operably linked to the regulatory RNA and comprising: (i) a nucleic acid sequence that has at least 80% identity to any one of SEQ ID NOs: 6-35, 43-77, and 79-81, and retains transcription initiation function; or (ii) a nucleic acid sequence of any one of SEQ ID NOs: 6-35, 43-77, and 79-81.

In some aspects, the present disclosure provides plants or plant parts that are produced by the method of the present disclosure, and comprise the DNA constructs of the present disclosure.

### BRIEF DESCRIPTION OF THE DRAWINGS

FIG. 1 depicts editing frequencies in pea protoplasts co-transfected with a guide RNA construct operably linked to a promoter set forth in the figure and a CRISPR-Cas12a (Cpf1) nuclease construct. The nucleic acid sequences of the PsUBI3p and control (AtUBI11p) promoters are set forth as SEQ ID NOs: 6 and 43, respectively. Unless specified in the figure, the guide RNA or the nuclease was operably linked to the control promoter.

FIG. 2 depicts editing frequencies in pea protoplasts co-transfected with a guide RNA construct and a nuclease construct each operably linked to a control promoter (AtUBI11p) or a test promoter set forth in the figure.

FIG. 3 depicts editing frequencies in pea leaves infiltrated with Agrobacterium tumefaciens comprising a guide RNA and a nuclease each operably linked to PsUBI3p or AtUBI11p as described in the figure.

FIG. 4 depicts editing frequencies in pea T0 plants stably transformed with a guide RNA and a nuclease each operably linked to PsUBI3p or AtUBI11p as described in the figure.

FIG. 5 depicts diagrams of PsUBI3p, PsUBI3-LIKEp, PsUBI10p, PsUBI3-SYN3p, and Ps UBI3-SYN27p. The sequences of these five promoters are set forth as SEQ ID NOs: 6, 7, 8, and 11, and 79, respectively.

FIG. 6 depicts editing frequencies in pea leaves infiltrated with Agrobacterium tumefaciens comprising a guide RNA and a CRISPR-Cas12a nuclease (Mc.2Cpf1 2C-NLS nuclease) each operably linked to the promoters described in the figure.

FIG. 7 depicts percentages of soybean T0 plants having >25% edits over total T0 plants screened. T0 plants were stably transformed with a guide RNA and a CRISPR-Cas12a (Cpf1) nuclease each operably linked to the promoters described in the figure.

FIG. 8 depicts the incidence of overall profiled editing events in T0 plants, which comprise the incidence of “fixed” edit events (i.e., a consistent insertion-deletion profile across proliferating tissue in a mid-development T0 plant), and the incidence of “unfixed” edits (i.e., an inconsistent insertion-deletion profile across the T0 plant tissue) in the T0 plants stably transformed with a guide RNA and a CRISPR-Cas12a nuclease each operably linked to the promoters described in the figure.

FIG. 9 depicts editing frequencies in soybean cotyledon protoplasts co-transfected with a guide RNA construct operably linked to a control promoter (AtUBI11p) and a nuclease construct operably linked to a test promoter as set forth in the figure.

### DETAILED DESCRIPTION OF THE INVENTION

The present disclosure now will be described more fully hereinafter. The disclosure may be embodied in many different forms and should not be construed as limited to the aspects set forth herein; rather, these aspects are provided so that this disclosure will satisfy applicable legal requirements.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

A “plant” refers to a whole plant, any part thereof, or a cell or tissue culture derived from a plant, comprising any of: whole plants, plant components or organs (e.g., leaves, stems, roots, embryos, pollen, ovules, seeds, grains, leaves, flowers, branches, fruit, pulp, juice, kernels, ears, cobs, husks, stalks, root tips, anthers, etc.), plant tissues, plant cell tissue cultures from which plants can be regenerated, plant calli, plant clumps, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants comprising the introduced polynucleotides are also within the scope of the invention. Further provided is a processed plant product (e.g., extract) or byproduct that retains one or more polynucleotides disclosed herein.

As used herein, a “subject plant or plant cell” is one in which genetic alteration, such as a mutation, has been effected as to a polynucleotide of interest, or is a plant or plant cell which is descended from a plant or cell so altered and which comprises the alteration. As used herein, the term “mutated” or “genetically modified” or “transgenic” or “transformed” or “edited” plants, plant cells, plant tissues, plant parts or seeds refers plants, plant cells, plant tissues, plant parts or seeds that have been mutated by the methods of the present disclosure to include one or more mutations (e.g., insertions, substitutions, or deletions) in the genomic sequence.

As used herein, a “control plant” or “control plant part” or “control cell” or “control seed” refers to a plant or plant part or plant cell or seed that has not been subject to the methods and compositions described herein. A “control” or “control plant” or “control plant part” or “control cell” or “control seed” provides a reference point for measuring changes in phenotype of the subject plant or plant cell. A control plant or plant cell may comprise, for example: (a) a control promoter with reference to the promoters of the present disclosure; (b) a wild-type plant or cell, i.e., of the same genotype as the starting material for the genetic alteration which resulted in the subject plant or cell; (c) a plant or plant cell of the same genotype as the starting material but which has been transformed with a null construct (i.e. with a construct which has no known effect on the trait of interest, such as a construct comprising a marker gene); (d) a plant or plant cell which is a non-transformed segregant among progeny of a subject plant or plant cell; (e) a plant or plant cell genetically identical to the subject plant or plant cell but which is not exposed to conditions or stimuli (e.g., sucrose) that would induce expression of the polynucleotide of interest; or (f) the subject plant or plant cell itself, under conditions in which the polynucleotide of interest is not expressed. In certain instances, a control plant of the present disclosure is grown under the same environmental conditions (e.g., same or similar temperature, humidity, air quality, soil quality, water quality, and/or pH conditions) as a subject plant described herein. Similarly, a control protein or control protein composition can refer to a protein or protein composition that is isolated or derived from a control plant. In specific embodiments, a control plant, plant part, or plant cell is a plant, plant part, or plant cell that comprises a control promoter molecule or does not comprise the promoter molecule of the present disclosure.

Plant cells possess nuclear, plastid, and mitochondrial genomes. The compositions and methods of the present invention may be used to modify the sequence of the nuclear, plastid, and/or mitochondrial genome, or may be used to modulate the expression of a gene or genes encoded by the nuclear, plastid, and/or mitochondrial genome. Accordingly, by “chromosome” or “chromosomal” is intended the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell.

As used herein, the term “gene” or “coding sequence”, herein used interchangeably, refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the term a “nucleic acid”, used interchangeably with a “nucleotide”, refers to a molecule consisting of a nucleoside and a phosphate that serves as a component of DNA or RNA. For instance, nucleic acids include adenine, guanine, cytosine, uracil, and thymine.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example, and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, guide RNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Accordingly, “plant with a mutation” or “plant part with a mutation” or “plant cell with a mutation” or “plant genome with a mutation” refers to a plant or plant part or plant cell or plant genome that contains a mutation (e.g., an insertion, a substitution, or a deletion) described in the present disclosure.

“Genome editing” or “gene editing” as used herein refers to a type of genetic engineering by which one or more mutations (e.g., insertions, substitutions, deletions, modifications) are introduced at a specific location of the genome. “Editing reagents”, as used herein, refers to a set of molecules or a construct comprising or encoding the molecules for introducing one or more mutations in the genome. Exemplary editing reagents comprise a nuclease and a guide RNA. For example, a CRISPR (clustered regularly interspaced short palindromic repeats) system comprises a CRISPR nuclease [e.g., CRISPR-associated (Cas) endonuclease or a variant thereof, such as Cas12a] and a guide RNA. A CRISPR nuclease associates with a guide RNA that directs nucleic acid cleavage by the associated endonuclease by hybridizing to a recognition site in a polynucleotide. The guide RNA comprises a direct repeat and a guide sequence, which is complementary to the target recognition site. In certain embodiments, the CRISPR system further comprises a tracrRNA (trans-activating CRISPR RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

As used herein, the terms “nuclease” and “endonuclease” are used interchangeably to refer to naturally-occurring or engineered enzymes, which cleave a phosphodiester bond within a polynucleotide chain.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory molecules and polynucleotides that are not found together in nature. For example, a recombinant DNA construct may comprise regulatory molecules and polynucleotides that are derived from different sources, or regulatory molecules and polynucleotides derived from the same source and arranged in a manner different than that found in nature. Such a construct may be used by itself or may be used in conjunction with a vector.

An expression construct can permit transcription of a particular polynucleic acid sequence in a host cell (e.g., a bacterial cell or a plant cell). An expression cassette may be part of a plasmid, viral genome, or nucleic acid fragment. Typically, an expression cassette includes a polynucleotide to be transcribed, operably linked to a promoter. “Operably linked” is intended to mean a functional linkage between two or more elements. For example, an operable linkage between a promoter of the present invention and a heterologous nucleotide is a functional link that allows for expression of the heterologous nucleic acid molecule. Operably linked elements may be contiguous or non-contiguous. When used to refer to the joining of two protein coding regions, by operably linked is intended that the coding regions are in the same reading frame. The cassette may additionally contain at least one additional polynucleotide to be co-transformed into the plant. Alternatively, the additional polynucleotide(s) can be provided on multiple expression cassettes or DNA constructs. Such an expression cassette or construct is provided with a plurality of restriction sites and/or recombination sites for insertion of the heterologous nucleotide sequence of interest to be under the transcriptional regulation of the promoter regions of the invention. The expression cassette may additionally contain selectable marker genes. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, “function” of a gene, a polynucleotide, a peptide, a protein, or a molecule refers to activity of a gene, a polynucleotide, a peptide, a protein, or a molecule. For example, the function of a guide RNA or a CRISPR nuclease may be assessed by editing efficiency of a target gene.

As used herein, the term “expression” or “expressing” refers to the transcription and/or translation of a particular nucleic acid sequence driven by a promoter.

“Introduced” in the context of inserting a nucleic acid molecule (e.g., a DNA construct comprising a promoter molecule and a polynucleotide sequence of interest) into a cell, a plant, or a plant part means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

As used herein with respect to a parameter, the term “decreased” or “decreasing” or “decrease” or “reduced” or “reducing” or “reduce” or “lower” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “decreased”, “reduced”, and the like encompass both a partial reduction and a complete reduction compared to a control.

As used herein with respect to a parameter, the term “increased” or “increasing” or “increase” or “enhanced” or “enhancing” or “enhance” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or 1000%) positive change in the parameter from a comparison control, e.g., an established normal or reference level of the parameter, or an established standard control.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleic acid sequence (cDNA), a genomic polynucleic acid sequence and/or a composite polynucleic acid sequences (e.g., a combination of the above).

The term “isolated” refers to at least partially separated from the natural environment e.g., from a plant cell.

As used herein, the terms “exogenous” or “heterologous” in reference to a nucleic acid sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the term “endogenous” in reference to a gene or nucleic acid or protein is intended to mean a gene or nucleic acid or protein that is naturally comprised within or expressed by a cell. Endogenous genes can include genes that naturally occur in the cell of a plant, but that have been modified in the genome of the cell without insertion or replacement of a heterologous gene that is from another plant species or another location within the genome of the modified cell.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

“Homolog” or “homologous sequence” may refer to both orthologous and paralogous sequences. Paralogous sequence relates to gene-duplications within the genome of a species. Orthologous sequence relates to homologous genes in different organisms due to ancestral relationship. Thus, orthologs are evolutionary counterparts derived from a single ancestral gene in the last common ancestor of given two species and therefore have great likelihood of having the same function. One option to identify homologs (e.g., orthologs) in monocot plant species is by performing a reciprocal BLAST search. This may be done by a first blast involving blasting the sequence-of-interest against any sequence database, such as the publicly available NCBI database which may be found at: ncbi.nlm.nih.gov. If orthologs in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An ortholog is identified when the sequence resulting in the highest score (best hit) in the first blast identifies in the second blast the query sequence (the original sequence-of-interest) as the best hit. Using the same rational a paralog (homolog to a gene in the same organism) is found. In case of large sequence families, the ClustalW program may be used [ebi.ac.uk/Tools/clustalw2/index.html], followed by a neighbor-joining tree (wikipedia.org/wiki/Neighbor-joining) which helps visualizing the clustering.

In some embodiments, the term “homolog” as used herein, refers to functional homologs of genes. A functional homolog is a gene encoding a polypeptide that has sequence similarity to a polypeptide encoded by a reference gene, and the polypeptide encoded by the homolog carries out one or more of the biochemical or physiological function(s) of the polypeptide encoded by the reference gene. In general, it is preferred that functional homologs and/or polypeptides encoded by functional homologs share at least some degree of sequence identity with the reference gene or polypeptide encoded by the reference gene. Homology (e.g., percent homology, sequence identity+sequence similarity) can be determined using any homology comparison software computing a pairwise sequence alignment.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. The determination of percent sequence identity between any two sequences can be accomplished using a mathematical algorithm, or a computer implementation thereof. Nonlimiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms for comparison of sequences to determine sequence identity include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection. Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence. According to some embodiments, the homology is a global homology, e.g., a homology over the entire amino acid or nucleic acid sequences of the invention and not over portions thereof. The degree of homology or identity between two or more sequences can be determined using various known sequence comparison tools which are described in WO2014/102774.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “population” refers to a set comprising any number, including one, of individuals, objects, or data from which samples are taken for evaluation, e.g., estimating quantitative trait locus (QTL) associations and/or disease tolerance. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silico representations of plants. The member of a population need not be identical to the population members selected for use in subsequent cycles of analyses, nor does it need to be identical to those population members ultimately selected to obtain a final progeny of plants. Often, a plant population is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., fiber lint percent, fiber quality, seed protein content, seed carbohydrate content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant's growth, development and/or performance, including agents that affect other organisms in the plant's environment when those effects subsequently alter a plant's performance, growth, and/or development (e.g. an insecticide that kills plant pathogens in the plant's environment, thereby improving the ability of the plant to tolerate the insect's presence). Agriculture treatment agents also include a broad range of chemicals and/or biological substances that are applied to seeds, in which case they are commonly referred to as seed treatments and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g., microencapsulation). In certain embodiments, the agriculture treatment agent comprises a nanoparticle and/or the application of the agriculture treatment agent comprises the use of nanotechnology. In some embodiments, the plants described herein can grow in the presence of one or more agricultural treatment agents. For example, the plants described herein can have an increased expression of the polynucleotide of interest, e.g., a guide RNA or a nuclease, or mutations in the genome introduced by such editing reagents, and can grow in the presence of commonly used herbicides.

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

All publications, patent applications, patents, and other references mentioned herein are incorporated by reference herein in their entirety.

### II. Overview of the Invention

Enhanced expression of a polynucleotide of interest in a plant or plant part can promote successful development of desirable traits. For instance, the efficiency of introducing mutations can be closely associated with expression levels and spatio-temporal expression patterns of reagents that are responsible for introducing a mutation. For example, following delivery of expression constructs, expression determines the level of bioavailable ribonucleoprotein complexes for introducing mutations using a CRISPR system. Methods and compositions for increasing CRISPR reagent expression have the potential to drive significant improvements in plant editing efficiencies, and types of mutation outcomes (such as homology-directed repair).

Accordingly, efficient promoters for expressing a polynucleotide of interest in plant cells are advantageous. Specifically, promoters for expressing a polynucleotide of interest in a legume embryonic axes have been sought. In the context of enabling routine high-level editing of crop plants, the meristems of the explants are a desirable target of transforming editing reagents as the meristems undergo editing concurrent with their growth into whole plants. It is preferred that regenerated plants contain a ubiquitous (heritable) editing outcome, rather than a mosaic pattern of editing events across somatic tissues or no detectable editing in T0 plants. Moreover, it is desired that the promoter and leader elements have an immediate, efficient induction of expression of operably linked editing reagents in transformed meristems to enable editing to at a rate faster than their cellular division.

Described herein are promoters, including 5′ untranslated regions (5′UTRs), for efficient expression and function of downstream polynucleotides of interest in a plant or plant part. Provided herein is a novel pea (Pisum sativum) UBIQUITIN 3 promoter (PsUBI3p) and PsUBI promoter homologs (e.g., PsUBI3-LIKEp, PsUBI10p AhUBI3p, AhUBI3-LIKEp, AhUBI4p, CaUBI3p, CaUBI4p, LaUBI3p, LaUBI4Ap, LaUBI4Bp, LaUBBp, LjUBI3p, LjUBI3-LIKEp, LjUBI4p, PaUBI3-LIKEp, PaUBI4p, PaUBBp, PlUBI3Ap, PlUBI3Bp, PlUBBp, PvUBI3p, PvUBI4p, PvUBBp, VuUBI3p, VuUBI4p, VuUBBp), that can enhance expression of operably linked polynucleotides of interest compared to control promoters (e.g., AtUBI11p). When operably linked to editing reagents, PsUBI3p and other PsUBI promoter homologs can enhance expression of editing reagents and editing frequency at target sites compared to control promoters.

Further, novel synthetic motif sequences that, when inserted in a promoter molecule, enhance expression or function of downstream polynucleotides of interest in a plant or plant part are provided, including synthetic motif sequences derived from PsUBI3-LIKEp and PsUBI10p. When one copy of the motif sequence(s) is inserted into a promoter molecule (e.g., PsUBI3p), expression or function of a downstream polynucleotide can be enhanced compared to inserted control construct. When more than one copies of the motif sequence(s) are inserted into a promoter molecule (e.g., PsUBI3p), expression or function of a downstream polynucleotide can be enhanced compared to when one or no copy of motif sequence(s) is inserted. Further, when one or more copies of both motif sequences are inserted into a promoter molecule, the expression or function of the downstream polynucleotide can be further enhanced compared to when one or more copies of only one motif sequence is inserted. When operably linked to editing reagents, a promoter with one or more copies of the synthetic motif sequences (e.g., the promoter PsUBI-SYN1p, PsUBI-SYN2p, or PsUBI-SYN3p) can enhance expression of editing reagents and editing frequency at target sites compared to a control promoter without the motif sequence(s) or fewer copies of the motif sequence(s).

The synthetic motif sequences and the promoter molecules of the present invention can be useful for expressing operably linked nucleotide sequences, e.g., in a constitutive matter and/or at any tissue in a plant or plant part, including but not limited to meristematic tissue. When operably linked to editing reagents (e.g., CRISPR-Cas12a reagents), the promoter molecules or those incorporating one or more of the synthetic motif sequences of the present invention can provide high frequencies of heritable genome editing in plants or plant parts.

### III. Nucleic Acid Molecules Comprising a Promoter Sequence

The present disclosure provides promoters, including 5′ untranslated regions (5′UTRs), for expression of downstream polynucleotides of interest in a plant or plant part. As used herein, “promoter” is intended to mean an upstream regulatory region of DNA prior to the ATG of a native gene, having a transcription initiation function for said gene and other downstream genes. As used herein “transcription initiation” refers to a phase during which the first nucleotides in the RNA chain are synthesized. It is a multistep process that starts with formation of a complex between a RNA polymerase holoenzyme and a DNA template at the promoter, and ends with dissociation of the core polymerase from the promoter after the synthesis of approximately first nine nucleotides. A promoter sequence can include a 5′ untranslated region (5′UTR), including intronic sequences, in addition to a core promoter that contains a TATA box capable of directing RNA polymerase II (pol II) to initiate RNA synthesis at the appropriate transcription initiation site for a particular polynucleotide sequence of interest. A promoter may additionally comprise other recognition sequences positioned upstream of the TATA box, and well as within the 5′UTR intron, which influence the transcription initiation rate.

In some aspects, the present disclosure provides nucleic acid molecules comprising a promoter sequence, wherein the promoter sequence has transcription initiation function and comprises one or more synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3. In some embodiments, the promoter sequence comprises at least two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3.

The synthetic motif sequence(s) can be inserted into any promoter molecule, including ubiquitin promoters such as PsUBI3p, PsUBI3-LIKEp, PsUBI10p, AhUBI3p, AhUBI3-LIKEp, AhUBI4p, CaUBI3p, CaUBI4p, LaUBI3p, LaUBI4Ap, LaUBI4Bp, LaUBBp, LjUBI3p, LjUBI3-LIKEp, LjUBI4p, PaUBI3-LIKEp, PaUBI4p, PaUBBp, PlUBI3Ap, PlUBI3Bp, PlUBBp, PvUBI3p, PvUBI4p, PvUBBp, VuUBI3p, VuUBI4p, VuUBBp, AtUBI10p (Peterson et al. 2016 PLoS ONE 11(9): e0162169; Tang et al. 2017 Nature Plants 3:17018; Curtin et al. 2018 Plant Biotechnol J. 16(6):1125-1137), AtUBQ1p (Mao et al. 2013 Mol Plant 6(6):2008-2011; Zhang et al. 2016 Plant Cell Rep. 35(7):1519-1533), GmUBIp (Curtin et al. 2018 Plant Biotechnol J. 16(6):1125-1137; Michno et al. 2020 BMC Biotechnology 20:10), OsUBI10p (Ding et al. 2018 Mol Plant 11(4):542-552; Li et al. 2019 Plant Biotechnol. J. 17(10):1862-1864), PcUBI4-2p (Fauser et al. 2014 Plant J. 79(2):348-359; Schindele et al. 2019 Plant Biotechnol. J. 18(5):1118-1120; Ordon et al. 2019 Functional & Integrative Genomics 20:151-162; Endo et al. 2016 Sci Rep. 6:38169), PvUBI1p (Cermak et al. 2017 Plant Cell 29(6):1196-1217), PvUBI2p (Cermak et al. 2017 Plant Cell 29(6):1196-1217), and ZmUBIp (Endo et al. 2016 Sci Rep. 6:38169; Sun et al. 2015 Scientific Reports 5:10342; Tang et al. 2016 Mol. Plant 9(7):1088-1091; Tang et al. 2017 Nature Plants 3:17018; Tang et al. 2019 Plant Biotechnol. J. 17(7):1431-1445; Malzahn et al. 2019 BMC Biol. 17:9; Wang et al. 2017 Mol. Plant 10(7):1011-1013; Wang et al. 2018 J. Integrat. Plant Biol. 60(8):626-631; Xu et al. 2019 Plant Biotechnol. J. 17(3):553-555; Zhong et al. 2018 Mol. Plant 11(7):999-1002; Zhong et al. 2020 Rice 13(8); Lee et al. 2018 Plant Biotechnol. J. 2019 17(2):362-372; Malzahn et al. 2019 BMC Biol. 17:9; Tang et al. 2017 Nature Plants 3:17018). Ps, Ah, Ca, La, Lj, Pa, Pl, Pv, Vu, At, Gm, and Zm represent molecules derived from Pisum sativum (pea), Arachis hypogaea (Peanut), Cicer arietinum (Chickpea), Lupinus albus (White lupin), Lotus japonicus, Phaseolus acutifolius (Tepary bean), Phaseolus lunatus (Lima bean), Phaseolus vulgaris (Common bean), Vigna unguiculata (Cowpea), Arabidopsis thaliana, Glycine max, and Zea mays, respectively. The synthetic motif sequence can be a sequence that is not present in the original promoter sequence to which the synthetic motif sequence is inserted (e.g. heterologous to the original promoter sequence). The synthetic motif sequence can be a sequence that shares less than 80% similarity with any part of the original promoter sequence to which the synthetic motif sequence is inserted. Alternatively, the synthetic motif sequence can be a sequence that shares at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence similarity with, or identical to, part of the original promoter sequence.

In some embodiments, the promoter sequence comprises one or more linkers. A “linker” as used herein refers to a nucleic acid molecule that connects two nucleic acid molecules or structures. In some embodiments, a linker connects the at least two synthetic motif sequences in the promoter. In some embodiments, a linker connects the promoter sequence and one of the synthetic motif sequence at the site of insertion. In some embodiments, the linker has a nucleic acid sequence that is about 1-100, 1-90, 1-80, 1-70, 1-60, 1-50, 1-40, 1-30, 1-20, 1-10, 1-5, 5-10, 10-15, 15-20, 20-25, 25-30, 35-40, 40-45, 45-50, 50-55, 55-60, 60-65, 65-70, 70-75, 75-80, 80-85, 85-90, 90-95, 95-100, or more than 100 (e.g., about 1, 2, 3, 4, 5, 6, 7, 8, 9, 10, 15, 20, 25, 30, 35, 40, 45, 50, 55, 60, 65, 70, 75, 80, 85, 90, 95, 100, or more) nucleotides in length. In a specific embodiment, the linker is 5 nucleotides in length. In a further embodiment, the linker comprises a nucleic acid sequence of ACGTA or TTATG, or a nucleic acid sequence that shares at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to ACGTA or TTATG. In a specific embodiment, the promoter sequence comprises the linker ACGTA connecting the promoter sequence and the first motif sequence, and the linker TTATG connecting the first motif sequence and the second motif sequence, and the linker C connecting the second motif sequence and the promoter sequence.

In some embodiments, the promoter sequence of the present disclosure comprises two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3. In some embodiments, said two synthetic motif sequences are different synthetic motif sequences. “Different” as used herein refers to being not identical or having sequence identity of less than 100%.

In some embodiments, said two or at least two synthetic motif sequences comprise: (a) a nucleic acid sequence that has at least 95% (95%, 96%, 97%, 98%, 99%, or 100%) sequence identity to SEQ ID NO: 1, or the nucleic acid sequence of SEQ ID NO: 1; and (b) a nucleic acid sequence that has at least 95% (95%, 96%, 97%, 98%, 99%, or 100%) sequence identity to SEQ ID NO: 2; or the nucleic acid sequence of SEQ ID NO: 2.

In some embodiments, said one or more synthetic motif sequences are inserted into the 5′UTR of said promoter sequence. In some embodiments, said one or more synthetic motif sequences are inserted into a 5′ intron, e.g., a mid-intron region of said promoter sequence.

Promoters disclosed herein comprising synthetic motif sequences, also referred to as synthetic promoters, can further incorporate specific mutations or clusters of mutations. As used herein, a “mutation cluster” or “cluster of mutations” or “mutation position cluster” refers to a region of the promoter comprising at least one mutation. The region of the promoter comprising the mutation cluster can be about 100, 120, 140, 160, 180, 200, 220, 240, 260, 280, or about 300 nucleotides. A mutation cluster can comprise 1, 2, 3, 4, 5, 6, 7, 8, 9, 10, 11, 12, 13, 14, 15, 16, 17, 18, 19, or 20 mutations. The promoters disclosed herein can have 1, 2, 3, 4, 5, 6, 7, 8, or 9 mutation clusters. The synthetic promoters can comprise mutations or mutation clusters that increase transcriptional activity, or any other activity of the promoter. The synthetic promoters can comprise 1, 2, 3, 4, 5, 6, 7, 8, 9, 10, 11, 12, 13, 14, 15, 16, 17, 18, 19, 20, 21, 22, 24, 26, 28, 30, 32, 34, 36, 37, 38, 39, 40, 41, 4, 43, 44, 45, 46, 47, 48, 49, 50, 55, 60, 65, 70, 80, 90, 100, or more total mutations and retain activity. In specific embodiments, the mutations or mutation clusters can be located in the 5′ UTR of the promoter, the 5′ UTR intron, or a combination of the 5′ UTR and the 5′ UTR intron. Mutations or mutation clusters can also be located anywhere in the promoter, including a 5′ region, center region, or 3′ region of the promoter. The promoters can have at least one mutation at the following positions, when aligned for maximum full-length (i.e., global) identity with the PsUBI3-SYN3p promoter: positions 167, 169, 170, 171, 176, 177, 178, 179, 180, 561, 562, 563, 570, 571, 572, 573, 574, 1446, 1607, 1608, 1609, 1611, 1617, 1618, 1619, 1706, 1707, 1708, 1709, 1716, 1718, 1719, 1894, 2229, 2230, 2231, 2232, 2237, 2238, 2239, 2240, and 2241. In some embodiments, the promoter sequence disclosed herein comprises at least one mutation cluster at positions selected the following position clusters: a) at least one of 167, 169, 170, 171, 176, 177, 178, 179, and 180; b) at least one of 561, 562, 563, 570, 571, 572, 573, 574; c) at least one of 1446, 1607, 1608, 1609, 1611, 1617, 1618, 1619; d) at least one of 1706, 1707, 1708, 1709, 1716, 1718, 1719, 1894; and e) at least one of 2229, 2230, 2231, 2232, 2237, 2238, 2239, 2240, 2241. Promoters disclosed herein can have mutations in at least 2, at least 3, at least 4, at least 5, at least 6, at least 7, at least 8, or at least 9, or all of the positions in each mutation cluster. In specific embodiments, the promoters disclosed herein can have at least 1, in at least 2, 3, 4, 5, 6, 7, 8, 9, 10, 12, 14, 16, 18, 20, 22, 24, 26, 28, 30, 32, 34, 36, 38, 40, 41, or all 42 of the following mutations: C167G, A169G, T170A, T171C, T176G, C177G, T178C, T179A, T180G, T561G, A562T, T563G, T570G, T571G, A572C, T573A, T574G, C1446G, T1607G, C1608T, A1609G, G1611C, T1617G, T1618C, T1619A, T1706G, G1707T, T1708G, C1709A, T1716G, G1718A, C1719G, C1894A, A2229T, A2230G, G2231A, G2232C, C2237G, C2238G, A2239C, T2240A, and T2241G. In some embodiments, the promoter sequence disclosed herein comprises at least one mutation cluster selected the following: a) at least one of C167G, A169G, T170A, T171C, T176G, C177G, T178C, T179A, T180G; b) at least one of T561G, A562T, T563G, T570G, T571G, A572C, T573A, T574G; c) at least one of C1446G, T1607G, C1608T, A1609G, G1611C, T1617G, T1618C, T1619A; d) at least one of T1706G, G1707T, T1708G, C1709A, T1716G, G1718A, C1719G, C1894A; and e) at least one of A2229T, A2230G, G2231A, G2232C, C2237G, C2238G, A2239C, T2240A, T2241G. In some embodiments, the mutations disclosed herein can increase the activity of a promoter in the absence of synthetic motifs. Thus, provided herein are promoters having mutations corresponding to at least 2, at least 3, at least 4, or all 5 of the mutation clusters when aligned for maximum identity with the PsUBI3-SYN3p promoter.

In some embodiments, the promoter sequence comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 6, further comprising the one or more copies of the one or more synthetic motif sequences of the present disclosure inserted therein; or (ii) the nucleic acid sequence of SEQ ID NO: 6, further comprising one or more copies of the synthetic motif sequences of the present disclosure inserted therein. In some embodiments, the promoter sequence comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 9-11 and 79-81 or (ii) the nucleic acid sequence of any one of SEQ ID NOs: 9-11 and 79-81. In specific embodiments the sequence identity is calculated outside of the nucleotide positions comprising mutations or mutation clusters described herein. Thus, provided herein are promoters having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOs: 9-11 and 79-81 and comprising at least one mutation or mutation cluster selected from: a) at least one of C167G, A169G, T170A, T171C, T176G, C177G, T178C, T179A, T180G; b) at least one of T561G, A562T, T563G, T570G, T571G, A572C, T573A, T574G; c) at least one of C1446G, T1607G, C1608T, A1609G, G1611C, T1617G, T1618C, T1619A; d) at least one of T1706G, G1707T, T1708G, C1709A, T1716G, G1718A, C1719G, C1894A; and e) at least one of A2229T, A2230G, G2231A, G2232C, C2237G, C2238G, A2239C, T2240A, T2241G.

The promoters of the invention can be used to express or enhance expression of any nucleic acid molecule of interest, such as any gene, polynucleotide, or regulatory element of interest. Eukaryotic promoters are complex and are comprised of components that include a TATA box consensus sequence at about 35 base pairs 5′ relative to the transcription start site or cap site which is defined as +1. The TATA motif is the site where the TATA-binding-protein (TBP) as part of a complex of several polypeptides (TFIID complex) binds and productively interacts (directly or indirectly) with factors bound to other sequence elements of the promoter. This TFIID complex in turn recruits the RNA polymerase II complex to be positioned for the start of transcription generally 25 to 30 base pairs downstream of the TATA element and promotes elongation thus producing RNA molecules. The sequences around the start of transcription (designated INR) of some pol I genes seem to provide an alternate binding site for factors that also recruit members of the TFIID complex and thus “activate” transcription. These INR sequences are particularly relevant in promoters that lack functional TATA elements providing the core promoter binding sites for eventual transcription. It has been proposed that promoters containing both a functional TATA and INR motif are the most efficient in transcriptional activity. (Zenzie-Gregory et al, 1992. J. Biol. Chem. 267:2823-2830). See, for example, U.S. Pat. No. 6,072,050, herein incorporated by reference.

The invention encompasses isolated or substantially purified polynucleotide or nucleic acid compositions. An “isolated” or “purified” polynucleotide, or biologically active portion thereof, is substantially or essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived.

Fragments and variants of the disclosed promoter molecules are also encompassed by the present invention. By “fragment” is intended a portion of the nucleic acid sequence. Variant sequences can be isolated by PCR as well as hybridization. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

The promoters of the invention can have a number of characteristics. The promoter may have a constitutive expression profile. In some aspects, the promoters of the present disclosure provide constitutive expression of an operably linked nucleotide of interest (e.g., encoding a guide RNA or nuclease). In some aspects, the promoters of the present disclosure provide increased constitutive expression of an operably linked polynucleotide of interest (e.g., guide RNA, nuclease) compared to a control promoter. The promoters of the present disclosure can have superior effects in expressing a polynucleotide of interest in a plant or plant part compared to constitutive promoters known in the art, e.g., the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like.

Additionally or alternatively, the promoters of the present disclosure can be tissue-preferred promoters. In some embodiments, the promoters of the present disclosure preferably target meristematic tissue. The promoters of the present disclosure can have superior effects in expressing a polynucleotide of interest in a plant or plant part compared to issue-preferred promoters described in the art, e.g., Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Additionally or alternatively, promoters of the present disclosure can be cell-preferred promoters. Such promoters may preferentially drive the expression of a downstream polynucleotide in a particular cell type such as a meristematic tissue cell. The promoters of the present disclosure can have superior effects in expressing a polynucleotide of interest in a plant or plant part compared to cell-preferred promoters described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like.

Additionally or alternatively, promoters of the present disclosure can be developmentally-regulated promoters. Such promoters may show a peak in expression at a particular developmental stage. The promoters of the present disclosure can have superior effects in expressing a polynucleotide of interest in a plant or plant part compared to developmentally-regulated promoters described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like.

Additionally or alternatively, promoters of the present disclosure can be promoters that are induced following the application of a particular biotic and/or abiotic stress. The promoters of the present disclosure can have superior effects in expressing a polynucleotide of interest in a plant or plant part compared to inducible promoters described in the art, e.g., Yi et al. (2010) Planta 232: 743-754; Yamaguchi-Shinozaki and Shinozaki (1993) Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

It is recognized that, in some instances, a specific, non-constitutive expression profile may provide an improved plant phenotype. For instance, many plant genes are regulated by light conditions, the application of particular stresses, the circadian cycle, or the stage of a plant's development. These expression profiles may be important for the function of the gene, polynucleotide, gene product, or polynucleotide product in planta. One strategy that may be used to provide a desired expression profile in combination with the promoters, compositions, or methods of the present disclosure is the use of synthetic promoters containing cis-regulatory elements that drive the desired expression levels at the desired time and place in the plant. The promoters of the present disclosure can comprise cis-regulatory elements that can be used to alter polynucleotide expression in planta. Further, the promoters of the present disclosure can have superior effects in expressing a polynucleotide of interest in a plant or plant part compared to promoters comprising cis-regulatory elements that have been described in the scientific literature, e.g., Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

Expression or function of an operably linked polynucleotide of interest (e.g., encoding an editing reagent such as a nuclease or a guide RNA) can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more with a promoter of the present disclosure, as compared to a control promoter. Expression of an operably linked polynucleotide of interest can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more with a promoter of the present disclosure compared to a control promoter. Polynucleotide expression levels can be measured by any methods known in the art. For example, polynucleotide expression levels can be measured by quantifying levels of the polynucleotide product, e.g., an RNA or a protein, by, e.g., PCR, real-time PCR, Western blotting, and ELISA. Polynucleotide expression levels can also be assessed by quantifying levels of function of polynucleotide product, for example by quantifying the occurrence of events caused by the polynucleotide product (e.g., editing frequency of a target gene) or by quantifying the levels of product produced by the polynucleotide product, as further disclosed elsewhere in the present disclosure.

### IV. Constructs Comprising a Promoter Operably Linked to a Polynucleotide of Interest

The present disclosure provides DNA constructs (e.g., expression constructs) comprising, in operable linkage, the promoter of the present disclosure and a polynucleotide of interest comprising a nucleotide sequence of interest (e.g., encoding a guide RNA or nuclease). DNA constructs can comprise, in operable linkage: (a) a nucleic acid molecule comprising a promoter sequence/molecule and (b) a polynucleotide of interest. The promoter sequence/molecule can have transcription initiation function and comprise one or more synthetic motif sequences, wherein each synthetic motif sequence comprises a nucleic acid sequence that has at least 95% (95%, 96%, 97%, 98%, 99%, or 100%) sequence identity to any one of SEQ ID NOs: 1-3 or) the nucleic acid sequence of any one of SEQ ID NOs: 1-3. Alternatively or additionally, the promoter sequence/molecule can comprise a Pisum sativum ubiquitin promoter (PsUBI promoter, e.g., PsUBI3p, PsUBI3-LIKEp, PsUBI10p) or a homolog of PsUBI promoters from other legumes such as Arachis hypogaea (peanut), Cicer arietinum (chickpea), Lupinus albus (white lupin), Lotus japonicus, Phaseolus acutifolius (Tepary bean), Phaseolus lunatus (Lima bean), Phaseolus vulgaris (Common bean), Vigna unguiculata (Cowpea), e.g., AhUBI3p, AhUBI3-LIKEp, AhUBI4p, CaUBI3p, CaUBI4p, LaUBI3p, LaUBI4Ap, LaUBI4Bp, LaUBBp, LjUBI3p, LjUBI3-LIKEp, LjUBI4p, PaUBI3-LIKEp, PaUBI4p, PaUBBp, PlUBI3Ap, PlUBI3Bp, PlUBBp, PvUBI3p, PvUBI4p, PvUBBp, VuUBI3p, VuUBI4p, VuUBBp. For example, the promoter sequence/molecule can comprise a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-8 and 12-35, and retains transcription initiation function or a nucleic acid sequence of any one of SEQ ID NOs: 6-8 and 12-35.

In some embodiments, the promoter molecule of the DNA construct comprises at least two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3.

In some embodiments, the DNA construct comprises one or more linkers. In some embodiments, a linker connects the at least two synthetic motif sequences in the promoter. In some embodiments, a linker connects the promoter sequence and one of the synthetic motif sequence at the site of insertion. In some embodiments, a linker connects the promoter molecule and the polynucleotide of interest. In some embodiments, the linker has a nucleic acid sequence that is about 1-100, 1-90, 1-80, 1-70, 1-60, 1-50, 1-40, 1-30, 1-20, 1-10, 1-5, 5-10, 10-15, 15-20, 20-25, 25-30, 35-40, 40-45, 45-50, 50-55, 55-60, 60-65, 65-70, 70-75, 75-80, 80-85, 85-90, 90-95, 95-100, or more than 100 (e.g., about 1, 2, 3, 4, 5, 6, 7, 8, 9, 10, 15, 20, 25, 30, 35, 40, 45, 50, 55, 60, 65, 70, 75, 80, 85, 90, 95, 100, or more) nucleotides in length. In a specific embodiment, the linker is 5 nucleotides in length. In a further embodiment, the linker comprises a nucleic acid sequence of ACGTA or TTATG, or a nucleic acid sequence that shares at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with ACGTA or TTATG. In a specific embodiment, the promoter molecule comprises the linker ACGTA connecting the promoter sequence and the first motif sequence, and the linker TTATG connecting the first motif sequence and the second motif sequence, and the linker C connecting the second motif sequence and the promoter sequence.

In some embodiments, the promoter molecule of the DNA construct comprises two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3. In some embodiments, said two synthetic motif sequences are different synthetic motif sequences.

In some embodiments, said two or at least two synthetic motif sequences comprise: (a) a nucleic acid sequence that has at least 95% (95%, 96%, 97%, 98%, 99%, or 100%) sequence identity to SEQ ID NO: 1, or the nucleic acid sequence of SEQ ID NO: 1; and (b) a nucleic acid sequence that has at least 95% (95%, 96%, 97%, 98%, 99%, or 100%) sequence identity to SEQ ID NO: 2; or the nucleic acid sequence of SEQ ID NO: 2. In some embodiments, said one or more synthetic motif sequences are inserted into the 5′UTR of said promoter sequence. In some embodiments, said one or more synthetic motif sequences are inserted into a 5′ intron, e.g., a mid-intron region, of said promoter sequence.

In some embodiments, the promoter molecule of the DNA construct comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 6, further comprising one or more copies of the one or more synthetic motif sequences of the present disclosure inserted therein; or (ii) the nucleic acid sequence of SEQ ID NO: 6, further comprising one or more copies of the one or more synthetic motif sequences of the present disclosure inserted therein. In some embodiments, the promoter molecule comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 9-11 and 79-81 or (ii) the nucleic acid sequence of any one of SEQ ID NOs: 9-11 and 79-81.

Promoter sequences disclosed herein as part of a construct and/or operably linked to a polynucleotide of interest can have mutations in at least 2, at least 3, at least 4, or all 5 of the following positions [[ ]]. In specific embodiments, the promoters disclosed herein can have at least 1, in at least 2, at least 3, at least 4, or all 5 of the following mutations: [[ ]].

In some embodiments, the polynucleotide of interest of the DNA construct of the present disclosure encodes a guide RNA or a nuclease. In some embodiments, the polynucleotide of interest of the DNA construct encodes a guide RNA and a nuclease, both operably linked to the promoter molecule of the DNA construct.

In some embodiments, the polynucleotide of interest of the DNA construct operably linked to the promoter molecule encodes a guide RNA, and the DNA construct further comprises, in operable linkage, a nucleic acid molecule encoding a second promoter molecule and a polynucleotide encoding a nuclease. In some embodiments, the second promoter molecule is an RNA polymerase II promoter molecule. As used herein, “RNA polymerase II” is an enzyme localized in the nucleoplasm and synthesizes precursors to mRNAs and some small nuclear RNAs (e.g., sRNAs, microRNAs). The second promoter molecule can have transcription initiation function and comprise: (i) one or more synthetic motif sequences, each synthetic motif sequence comprising a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; (ii) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-43 and 79-81, and retains transcription initiation function, or a nucleic acid sequence of any one of SEQ ID NOs: 6-43 and 79-81.

In some embodiments, the polynucleotide of interest of the DNA construct operably linked to the promoter molecule encodes a nuclease, and the DNA construct further comprises, in operable linkage: a nucleic acid molecule encoding a second promoter molecule and a polynucleotide encoding a guide RNA. In some embodiments, the second promoter molecule is an RNA polymerase III (pol III) promoter molecule. As used herein, “RNA polymerase III” is an enzyme that transcribes 5S rRNA, tRNA, and some small nuclear RNA genes in the nucleus and cytosol. The second promoter molecule can have transcription initiation function and comprise: (i) one or more synthetic motif sequences, each comprising a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; or (ii) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-35, 43-77, and 79, and retains transcription initiation function or a nucleic acid sequence of any one of SEQ ID NOs: 6-35, 43-77, and 79-81. The nucleases and gRNAs that can be expressed using the promoters or constructs of the present disclosure are further disclosed below.

In some embodiments, the DNA construct of the present disclosure further comprises, in operable linkage, a nucleic acid sequence encoding a selectable marker and/or a regulatory RNA. The selectable marker and/or the regulatory RNA can be operably linked to the promoter of the present disclosure, which can be the same promoter as the one to which said polynucleotide of interest (e.g., for a gRNA or a nuclease) is operably linked, or a different promoter, or any other promoter. In some embodiments, the regulatory RNA is operably linked to a promoter molecule comprising: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) identity to any one of SEQ ID NOs: 6-35, 43-77, and 79-81, and has transcription initiation function; or (ii) a nucleic acid sequence of any one of SEQ ID NOs: 6-35, 43-77, and 79-81.

The promoters disclosed herein can be operably linked to any polynucleotide of interest. As used herein, the term “polynucleotide of interest” can be interchangeably with the terms “coding sequence” or “nucleotide sequence of interest”. Polynucleotides of interest that are suitable for use in the present disclosed constructs include, but are not limited to, polynucleotides of interest that confer resistance to pests or disease, tolerance to herbicides, value added agronomic traits, such as yield improvement, nitrogen use efficiency, water use efficiency, and nutritional quality, binding of a protein to DNA in a site-specific manner, expression of small RNA, and selectable markers. In accordance with certain embodiments, the polynucleotide sequence of interest can encode a selectable marker or a gene product conferring insecticidal resistance, herbicide tolerance, small RNA expression, nitrogen use efficiency, water use efficiency, or nutritional quality.

Various insect resistance genes can be operably linked to the promoters disclosed herein. As examples of insect resistance genes that can be operably linked to the regulatory elements of the subject disclosure, the following traits are provided. Genes that provide exemplary Lepidopteran insect resistance include: cry1A; cry1A.105; cry1Ab; cry1Ab(truncated); cry1Ab Ac (fusion protein); cry1Ac; cry1C; cry1F; cry1Fa2; cry2Ab2; cry2Ae; cry9C; mocry1F; pinII (protease inhibitor protein); vip3A(a); and vip3Aa20. Genes that provide exemplary Coleopteran insect resistance include: cry34Ab1; cry35Ab1; cry3A; cry3Bb1; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

Various herbicide tolerance genes can be operably linked to the promoters disclosed herein. As examples of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to any promoters disclosed herein. For example, selectable marker genes include, but are not limited to genes encoding glyphosate resistance genes such as: mutant EPSPS genes including 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). Resistance genes for glufosinate and/or bialaphos compounds include dsm-2, bar and pat genes. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyri di noxy or phenoxy proprionic acids and cyclohexones include the ACCase inhibitor-encoding genes (e.g., Accl-S1, Accl-S2 and Accl-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1s+ genes (tolerance to triazine), and nitrilase genes (tolerance to benzonitrile). The above list of herbicide tolerance genes is not meant to be limiting. Any herbicide tolerance genes are encompassed by the present disclosure.

Various agronomic trait genes can be operably linked to the promoters disclosed herein. As examples of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

Various small RNA sequences can be operably linked to the promoters disclosed herein. As examples of small RNA coding sequences that can be operably linked to the regulatory elements of the subject disclosure, the following traits are provided. For example, delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the black spot bruise tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pR1 small RNA (degrades R1 transcripts to limit the formation of reducing sugars through starch degradation). Additional, benefits such as reduced acrylamide resulting from the asnl small RNA that triggers degradation of Asnl to impair asparagine formation and reduce polyacrylamide. Finally, the non-browning phenotype of pgas ppo suppression small RNA results in suppressing PPO to produce apples with a non-browning phenotype. The above list of small RNAs is not meant to be limiting. Any small RNA encoding sequences are encompassed by the present disclosure.

Various selectable markers also described as reporter genes can be operably linked to the promoters disclosed herein Examples of reporter genes encode: beta-glucuronidase (GUS), luciferase, green fluorescent protein (GFP), yellow fluorescent protein (YFP, Phi-YFP), red fluorescent protein (DsRFP, RFP, etc), beta-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for selection of transformed cells or tissues. Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

Herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Accl-S1, Accl-S2 and Accl-S3. Herbicides can also inhibit photosynthesis, including triazine (psbA and 1s+ genes) or benzonitrile (nitrilase gene). Furthermore, such selectable markers can include positive selection markers such as phosphomannose isomerase (PMI) enzyme.

Selectable marker genes can further include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). Selectable marker genes can further include genes encoding resistance to: chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

The polynucleotides of interest can be synthesized for optimal expression in a plant. For example, a polynucleotide of interest can have been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. For example, a polynucleotide of interest, e.g., a coding sequence, gene, heterologous coding sequence, or transgene/heterologous coding sequence can be designed to be expressed in plants at a higher level resulting in higher transformation efficiency. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

### A. Promoter Combinations in Constructs Comprising Nuclease and Guide RNA

In some embodiments of the present disclosure, the DNA construct comprises a nuclease coding sequence operably linked to a promoter molecule of the present disclosure and a polynucleotide encoding a guide RNA operably linked to the same or a second promoter. In some embodiments, the DNA construct comprises a polynucleotide encoding a guide RNA operably linked to a promoter molecule of the present disclosure and a polynucleotide encoding a nuclease operably linked to the same or a second promoter. In these embodiments, exemplary combinations of promoters for a nuclease and a guide RNA coding sequences are indicated with “X” in Table 1 below. Each promoter in Table 1 is meant to include its variants, e.g., promoter molecules that share at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence similarity with the promoter molecule indicated. Combinations are not limited to those listed herein. Any other sequences and combinations of promoters may be included in the DNA constructs, in addition to any variations to other components of the DNA constructs, according to the present disclosure.

TABLE 1
Promoters for Nuclease and Guide RNA Coding Sequences

| | | Promoter | | | | | | | | |
|---|---|---|---|---|---|---|---|---|---|---|
| | | comprising | | | | | | | | |
| | | one or more | Promoter Operably Linked to Nuclease | | | | | | | |
| | | copies of | | | | | | | | Any one |
| | | synthetic | | | | | | | | of PsUBI |
| | | motif | | PsUBI3- | | PsUBI3- | PsUBI3- | PsUBI3- | PsUBI3- | promoter |
| | | sequence | PsUBI3p | LIKEp | PsUBI10p | SYN1p | SYN2p | SYN3p | SYN27p | homologs |
| | | SEQ ID NOs: | (SEQ ID | (SEQ ID | (SEQ ID | (SEQ ID | (SEQ ID | (SEQ ID | (SEQ ID | (SEQ ID |
| | | 1, 2 and/or 3 | NO: 6) | NO: 7) | NO: 8) | NO: 9) | NO: 10) | NO: 11) | NO: 79) | NOs: 12-35) |
| Promoter | Promoter comprising | X | X | X | X | X | X | X | X | X |
| Operably | one or more copies | | | | | | | | | |
| Linked | of synthetic motif | | | | | | | | | |
| to Guide | sequence SEQ ID | | | | | | | | | |
| RNA | NOs: 1, 2, and/or 3 | | | | | | | | | |
| | PsUBI3p | X | X | X | X | X | X | X | X | X |
| | (SEQ ID NO: 6) | | | | | | | | | |
| | PsUBI3-LIKEp | X | X | X | X | X | X | X | X | X |
| | (SEQ ID NO: 7) | | | | | | | | | |
| | PsUBI10p | X | X | X | X | X | X | X | X | X |
| | (SEQ ID NO: 8) | | | | | | | | | |
| | PsUBI3-SYN1p | X | X | X | X | X | X | X | X | X |
| | (SEQ ID NO: 9) | | | | | | | | | |
| | PsUBI3-SYN2p | X | X | X | X | X | X | X | X | X |
| | (SEQ ID NO: 10) | | | | | | | | | |
| | PsUBI3-SYN3p | X | X | X | X | X | X | X | X | X |
| | (SEQ ID NO: 11) | | | | | | | | | |
| | PsUBI3-SYN27p | X | X | X | X | X | X | X | X | X |
| | (SEQ ID NO: 79) | | | | | | | | | |
| | Any one of PsUBI | X | X | X | X | X | X | X | X | X |
| | promoter homologs | | | | | | | | | |
| | (SEQ ID NOs: 12-35) | | | | | | | | | |
| | AtUBI11p | X | X | X | X | X | X | X | X | X |
| | (SEQ ID NO: 43) | | | | | | | | | |
| | AtU6-26p | X | X | X | X | X | X | X | X | X |
| | (SEQ ID NO: 44) | | | | | | | | | |
| | Mt7SLp | X | X | X | X | X | X | X | X | X |
| | (SEQ ID NO: 45) | | | | | | | | | |
| | Ps7SL2-1p | X | X | X | X | X | X | X | X | X |
| | (SEQ ID NO: 46) | | | | | | | | | |
| | Ps7SL4-1p | X | X | X | X | X | X | X | X | X |
| | (SEQ ID NO: 47) | | | | | | | | | |
| | Ps7SL4-2p | X | X | X | X | X | X | X | X | X |
| | (SEQ ID NO: 48) | | | | | | | | | |
| | Ps7SL6-1p | X | X | X | X | X | X | X | X | X |
| | (SEQ ID NO: 49) | | | | | | | | | |
| | Ps7SL7-1p | X | X | X | X | X | X | X | X | X |
| | (SEQ ID NO: 50) | | | | | | | | | |
| | Ps7SL7-2p | X | X | X | X | X | X | X | X | X |
| | (SEQ ID NO: 51) | | | | | | | | | |
| | Ps7SL7-3p | X | X | X | X | X | X | X | X | X |
| | (SEQ ID NO: 52) | | | | | | | | | |
| | Ps7SL7-4p | X | X | X | X | X | X | X | X | X |
| | (SEQ ID NO: 53) | | | | | | | | | |
| | PsU6-Ap | X | X | X | X | X | X | X | X | X |
| | (SEQ ID NO: 54) | | | | | | | | | |
| | PsU6-Bp | X | X | X | X | X | X | X | X | X |
| | (SEQ ID NO: 55) | | | | | | | | | |
| | PsU6-Cp | X | X | X | X | X | X | X | X | X |
| | (SEQ ID NO: 56) | | | | | | | | | |
| | PsU6-Dp | X | X | X | X | X | X | X | X | X |
| | (SEQ ID NO: 57) | | | | | | | | | |
| | PsU6-Ep | X | X | X | X | X | X | X | X | X |
| | (SEQ ID NO: 58) | | | | | | | | | |
| | MtU3-Bp | X | X | X | X | X | X | X | X | X |
| | (SEQ ID NO: 59) | | | | | | | | | |
| | PsU3B-1p | X | X | X | X | X | X | X | X | X |
| | (SEQ ID NO: 60) | | | | | | | | | |
| | PsU3B-2p | X | X | X | X | X | X | X | X | X |
| | (SEQ ID NO: 61) | | | | | | | | | |
| | PsU3B-3p | X | X | X | X | X | X | X | X | X |
| | (SEQ ID NO: 62) | | | | | | | | | |
| | PsU3B-4p | X | X | X | X | X | X | X | X | X |
| | (SEQ ID NO: 63) | | | | | | | | | |
| | PsU3B-5p | X | X | X | X | X | X | X | X | X |
| | (SEQ ID NO: 64) | | | | | | | | | |
| | PsU3B-6p | X | X | X | X | X | X | X | X | X |
| | (SEQ ID NO: 65) | | | | | | | | | |
| | PsU3B-7p | X | X | X | X | X | X | X | X | X |
| | (SEQ ID NO: 66) | | | | | | | | | |
| | PsU3B-8p | X | X | X | X | X | X | X | X | X |
| | (SEQ ID NO: 67) | | | | | | | | | |
| | PsU3B-9p | X | X | X | X | X | X | X | X | X |
| | (SEQ ID NO: 68) | | | | | | | | | |
| | PsU3B-10p | X | X | X | X | X | X | X | X | X |
| | (SEQ ID NO: 69) | | | | | | | | | |
| | PsU3B-11p | X | X | X | X | X | X | X | X | X |
| | (SEQ ID NO: 70) | | | | | | | | | |
| | PsU6-Fp | X | X | X | X | X | X | X | X | X |
| | (SEQ ID NO: 71) | | | | | | | | | |
| | PsU6-Hp | X | X | X | X | X | X | X | X | X |
| | (SEQ ID NO: 72) | | | | | | | | | |
| | MtU6-4p | X | X | X | X | X | X | X | X | X |
| | (SEQ ID NO: 73) | | | | | | | | | |
| | GmU6-Cp | X | X | X | X | X | X | X | X | X |
| | (SEQ ID NO: 74) | | | | | | | | | |
| | GmU6-Ip | X | X | X | X | X | X | X | X | X |
| | (SEQ ID NO: 75) | | | | | | | | | |
| | AtU3-Bp | X | X | X | X | X | X | X | X | X |
| | (SEQ ID NO: 76) | | | | | | | | | |
| | Gm7SLp | X | X | X | X | X | X | X | X | X |
| | (SEQ ID NO: 77) | | | | | | | | | |

| | | Promoter Operably Linked to Nuclease | | | | | | | |
|---|---|---|---|---|---|---|---|---|---|
| | | AtUBI10p | GmUBIp | GmYAO-1p | AtRPS5Ap | GmDMC1p | GmScreamM4p | 35S-ENp | AtUBI11p |
| | | (SEQ ID | (SEQ ID | (SEQ ID | (SEQ ID | (SEQ ID | (SEQ ID | (SEQ ID | (SEQ ID |
| | | NO: 36) | NO: 37) | NO: 38) | NO: 39) | NO: 40) | NO: 41) | NO: 42) | NO: 43) |
| Promoter | Promoter comprising | X | X | X | X | X | X | X | X |
| Operably | one or more copies | | | | | | | | |
| Linked | of synthetic motif | | | | | | | | |
| to Guide | sequence SEQ ID | | | | | | | | |
| RNA | NOs: 1, 2, and/or 3 | | | | | | | | |
| | PsUBI3p | X | X | X | X | X | X | X | X |
| | (SEQ ID NO: 6) | | | | | | | | |
| | PsUBI3-LIKEp | X | X | X | X | X | X | X | X |
| | (SEQ ID NO: 7) | | | | | | | | |
| | PsUBI10p | X | X | X | X | X | X | X | X |
| | (SEQ ID NO: 8) | | | | | | | | |
| | PsUBI3-SYN1p | X | X | X | X | X | X | X | X |
| | (SEQ ID NO: 9) | | | | | | | | |
| | PsUBI3-SYN2p | X | X | X | X | X | X | X | X |
| | (SEQ ID NO: 10) | | | | | | | | |
| | PsUBI3-SYN3p | X | X | X | X | X | X | X | X |
| | (SEQ ID NO: 11) | | | | | | | | |
| | PsUBI3-SYN27p | X | X | X | X | X | X | X | X |
| | (SEQ ID NO: 79) | | | | | | | | |
| | Any one of PsUBI | X | X | X | X | X | X | X | X |
| | promoter homologs | | | | | | | | |
| | (SEQ ID NOs: 12-35) | | | | | | | | |
| | AtUBI11p | | | | | | | | |
| | (SEQ ID NO: 43) | | | | | | | | |
| | AtU6-26p | | | | | | | | |
| | (SEQ ID NO: 44) | | | | | | | | |
| | Mt7SLp | | | | | | | | |
| | (SEQ ID NO: 45) | | | | | | | | |
| | Ps7SL2-1p | | | | | | | | |
| | (SEQ ID NO: 46) | | | | | | | | |
| | Ps7SL4-1p | | | | | | | | |
| | (SEQ ID NO: 47) | | | | | | | | |
| | Ps7SL4-2p | | | | | | | | |
| | (SEQ ID NO: 48) | | | | | | | | |
| | Ps7SL6-1p | | | | | | | | |
| | (SEQ ID NO: 49) | | | | | | | | |
| | Ps7SL7-1p | | | | | | | | |
| | (SEQ ID NO: 50) | | | | | | | | |
| | Ps7SL7-2p | | | | | | | | |
| | (SEQ ID NO: 51) | | | | | | | | |
| | Ps7SL7-3p | | | | | | | | |
| | (SEQ ID NO: 52) | | | | | | | | |
| | Ps7SL7-4p | | | | | | | | |
| | (SEQ ID NO: 53) | | | | | | | | |
| | PsU6-Ap | | | | | | | | |
| | (SEQ ID NO: 54) | | | | | | | | |
| | PsU6-Bp | | | | | | | | |
| | (SEQ ID NO: 55) | | | | | | | | |
| | PsU6-Cp | | | | | | | | |
| | (SEQ ID NO: 56) | | | | | | | | |
| | PsU6-Dp | | | | | | | | |
| | (SEQ ID NO: 57) | | | | | | | | |
| | PsU6-Ep | | | | | | | | |
| | (SEQ ID NO: 58) | | | | | | | | |
| | MtU3-Bp | | | | | | | | |
| | (SEQ ID NO: 59) | | | | | | | | |
| | PsU3B-1p | | | | | | | | |
| | (SEQ ID NO: 60) | | | | | | | | |
| | PsU3B-2p | | | | | | | | |
| | (SEQ ID NO: 61) | | | | | | | | |
| | PsU3B-3p | | | | | | | | |
| | (SEQ ID NO: 62) | | | | | | | | |
| | PsU3B-4p | | | | | | | | |
| | (SEQ ID NO: 63) | | | | | | | | |
| | PsU3B-5p | | | | | | | | |
| | (SEQ ID NO: 64) | | | | | | | | |
| | PsU3B-6p | | | | | | | | |
| | (SEQ ID NO: 65) | | | | | | | | |
| | PsU3B-7p | | | | | | | | |
| | (SEQ ID NO: 66) | | | | | | | | |
| | PsU3B-8p | | | | | | | | |
| | (SEQ ID NO: 67) | | | | | | | | |
| | PsU3B-9p | | | | | | | | |
| | (SEQ ID NO: 68) | | | | | | | | |
| | PsU3B-10p | | | | | | | | |
| | (SEQ ID NO: 69) | | | | | | | | |
| | PsU3B-11p | | | | | | | | |
| | (SEQ ID NO: 70) | | | | | | | | |
| | PsU6-Fp | | | | | | | | |
| | (SEQ ID NO: 71) | | | | | | | | |
| | PsU6-Hp | | | | | | | | |
| | (SEQ ID NO: 72) | | | | | | | | |
| | MtU6-4p | | | | | | | | |
| | (SEQ ID NO: 73) | | | | | | | | |
| | GmU6-Cp | | | | | | | | |
| | (SEQ ID NO: 74) | | | | | | | | |
| | GmU6-Ip | | | | | | | | |
| | (SEQ ID NO: 75) | | | | | | | | |
| | AtU3-Bp | | | | | | | | |
| | (SEQ ID NO: 76) | | | | | | | | |
| | Gm7SLp | | | | | | | | |
| | (SEQ ID NO: 77) | | | | | | | | |

In some specific embodiments, the DNA construct of the present disclosure comprises:
PsUBI3-SYN3p operably linked to a polynucleotide encoding a nuclease and a gRNA;
PsUBI3-SYN3p operably linked to a polynucleotide encoding a nuclease and AtU6-26p operably linked to a polynucleotide encoding a gRNA;
PsUBI3-SYN3p operably linked to a polynucleotide encoding a nuclease and PsUBI3p operably linked to a polynucleotide encoding a gRNA;
PsUBI3-SYN3p operably linked to a polynucleotide encoding a nuclease and AtUBI11p operably linked to a polynucleotide encoding a gRNA;

In some specific embodiments, the DNA construct of the present disclosure comprises:
PsUBI3-SYN27p operably linked to a polynucleotide encoding a nuclease and a gRNA;
PsUBI3-SYN27p operably linked to a polynucleotide encoding a nuclease and AtU6-26p operably linked to a polynucleotide encoding a gRNA;
PsUBI3-SYN27p operably linked to a polynucleotide encoding a nuclease and PsUBI3p operably linked to a polynucleotide encoding a gRNA;
PsUBI3-SYN27p operably linked to a polynucleotide encoding a nuclease and AtUBI11p operably linked to a polynucleotide encoding a gRNA;
PsUBI3 operably linked to a polynucleotide encoding a nuclease and PsUBI3-SYN3p operably linked to a polynucleotide encoding a gRNA;
PsUBI3p operably linked to a polynucleotide encoding a nuclease and a gRNA;
PsUBI3p operably linked to a polynucleotide encoding a nuclease, and AtUBI11p operably linked to a polynucleotide encoding a gRNA;
AtUBI11p operably linked to a polynucleotide encoding a nuclease, and PsUBI3-SYN3p operably linked to a polynucleotide encoding a gRNA; or
AtUBI11p operably linked to a polynucleotide encoding a nuclease, and PsUBI3p operably linked to a polynucleotide encoding a gRNA.

### B. Exemplary Molecules Included in Constructs of Present Disclosure

The promoters and DNA constructs of the present disclosure can be used to express or enhance expression of any polynucleotide of interest. The present disclosure is not limited to exemplary coding sequences or polynucleotides of interest that can be operably linked to the promoter molecule or DNA constructs of the present disclosure discussed herein.

Polynucleotides of interest can include editing reagents for editing any gene or genomic site of interest. As used herein, “editing reagents” refer to a set of molecules or a construct comprising or encoding the molecules for introducing one or more mutations in the genome, including a nuclease and a guide RNA. For example, editing reagents can be CRISPR reagents, TALEN reagents, and ZFN reagents. A nuclease can be a nickase, an endonuclease, a meganuclease, or a nuclease fusion. CRISPR reagents comprise a CRISPR nuclease (e.g., Cas endonuclease or a variant thereof, such as Cas12a) and a guide RNA. In certain embodiments, the CRISPR components further comprise a tracrRNA (trans-activating CRISPR RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease. Editing reagents can also include base editing components. For example, cytosine base editing (CBE) reagents, which change a C-G base pair to a T-A base pair, comprise a single guide RNA, a nuclease (e.g., dCas9, CAS9 nickase), a cytidine deaminase (e.g., APOBEC1), and a uracil DNA glycosylase inhibitor (UGI). Adenine base editing (ABE) reagents, which change an A-T base pair to a G-C base pair comprise a deaminase, (TadA), a nuclease (e.g., dCas or Cas nickase), and a guide RNA. Exemplary molecules useful for creating mutations in the genome of plant or plant part (i.e., editing reagents) that can be included in the DNA constructs of the present disclosure and/or operably linked to the promoter molecules of the present disclosure are set forth below.

### 1. Nuclease

The promoters of the present disclosure may be operably linked to nuclease sequences. The DNA constructs of the present disclosure may comprise nuclease sequences. Nucleases that can be used in the present disclosure in precise genome-editing technologies to modulate the expression of the endogenous sequence include, but are not limited to, CRISPR nucleases, including Cas9, Cas12a (Cpf1), Cms1 or any CRISPR endonuclease, including CRISPR nickases and nuclease-dead CRISPR nucleases (e.g., a deactivated Cas9, Cas12a, or Cms1 endonuclease); meganucleases designed against the plant genomic sequence of interest (D'Halluin et al (2013) Plant Biotechnol J 11: 933-941); transcription activator-like effector nucleases (TALENs); or zinc finger nucleases (ZFNs).

In some embodiments, the nuclease encoded by the coding sequence of the DNA construct is a clustered, regularly interspaced, short palindromic repeats (CRISPR)-associated Cas endonuclease. In specific embodiments, the CRISPR nuclease is a Cas12a nuclease, herein used interchangeably with a Cpf1 nuclease. In a specific embodiment, the Cas12a nuclease is a McCpf1 nuclease, e.g., a Mc.2Cpf1 2C-NLS nuclease. In some embodiments, the CRISPR nuclease shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 78 or comprises the sequence of SEQ ID NO: 78. In some embodiments, the nuclease is further operably linked to one or more nuclear localization sequences (NLSs) and/or one or more epitope tags.

### 2. Guide RNA

The promoters of the present disclosure may be operably linked to coding sequences for guide RNAs. The DNA constructs of the present disclosure may comprise coding sequences for guide RNAs. To introduce one or more mutations into a gene or a genomic site of interest, antisense constructions, complementary to at least a portion of the messenger RNA (mRNA) for the sequences of the gene or the genomic site of interest can be constructed. Antisense nucleotides are designed to hybridize with the corresponding mRNA. Modifications of the antisense sequences may be made as long as the sequences hybridize to and interfere with expression of the corresponding mRNA. In this manner, antisense constructions having at least 75%, optimally 80%, more optimally 85%, 90%, 95% or greater sequence identity to the corresponding sequences to be edited may be used. Furthermore, portions of the antisense nucleotides may be used to disrupt the expression of the target gene.

In some instances, a guide RNA may comprise a targeting region that is complementary to a targeted sequence as well as another region that allows the guide RNA to form a complex with a nuclease (e.g., a CRISPR nuclease) of interest. The targeting region of a guide RNA for use in the method described herein above may be 10-40 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long). For example, the targeting region of a guide RNA for use in the method described hereinabove may be 24 nucleotides in length.

### 3. Regulatory RNA

The promoters of the present disclosure may be operably linked to coding sequences for regulatory RNAs. The DNA constructs of the present disclosure may comprise coding sequences for regulatory RNAs. As used herein, a “regulatory RNA” refers to a non-coding RNA that regulates expression of genes. Regulatory RNAs comprise a heterogeneous group of short and long RNAs, including microRNA (miRNA) and long non-coding RNA (lncRNA).

### 4. Reporter Genes Selectable Marker Genes

The promoters of the present disclosure may be operably linked to reporter gene or selectable marker gene sequences. The DNA constructs of the present disclosure may comprise reporter gene or selectable marker gene sequences. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987)Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other selectable marker genes that could be employed on the expression constructs disclosed herein include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

### 5. Transfer DNA

The recombinant DNA constructs of the present disclosure may contain T-DNA sequences. For example, a recombinant DNA construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a guide RNA of the present disclosure into nuclear DNA genome of a host plant (e.g., a pea plant).

### 6. Regulatory Signals

In some embodiments, a recombinant DNA construct described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

### 7. Terminators

A transcription terminator may also be included in the expression cassettes of DNA constructs of the present invention. Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

### C. Vectors and Cells Comprising the Promoter or the Construct

In some aspects, further disclosed herein are vectors containing DNA constructs of the present disclosure. As used herein, “vector” refers to a nucleotide molecule (e.g., a plasmid, cosmid), bacterial phage, or virus for introducing a nucleotide construct, for example, a recombinant DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance. In some specific embodiments, provided herein are expression cassettes located on a vector comprising the promoter molecule of the present disclosure operably linked to a coding sequence (e.g., for a nuclease or a guide RNA).

In some embodiments, a vector is a plasmid containing a recombinant DNA construct of the present disclosure.

In some embodiments, a vector is a recombinant virus containing a recombinant DNA construct of the present disclosure. A recombinant virus described herein can be a recombinant lentivirus, a recombinant retrovirus, a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

In some aspects, the present disclosure provides cells comprising a nucleic acid molecule (comprising a promoter sequence) of the present disclosure or a DNA construct of the present disclosure. In some embodiments, the cell is selected from the group consisting of a plant cell, a bacterial cell, and a fungal cell. For example, the present disclosure provides a bacterium, e.g., an Agrobacterium tumefaciens, containing a promoter molecule of the present disclosure or a DNA construct of the present disclosure for expressing a polynucleotide of interest, e.g., editing reagents for genomic loci of interest. The cells of the present disclosure may be grown, or have been grown, in a cell culture.

### V. Plants Comprising a Heterogeneous Promoter and a Polynucleotide of Interest

Disclosed herein are plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products (e.g., plant extract, plant concentrate, plant powder, plant protein, and plant biomass) comprising the nucleic acid molecule (comprising the promoter sequence), the DNA construct, or the cell of the present disclosure. Also disclosed herein plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products (e.g., plant extract, plant concentrate, plant powder, plant protein, and plant biomass) generated by introducing the nucleic acid molecule (comprising the promoter sequence), the DNA construct, or the cell of the present disclosure into the plants or plant parts.

In some aspects, the present disclosure provides plants or plant parts comprising a DNA construct comprising, in operable linkage (a) a nucleic acid molecule comprising a promoter sequence and (b) a coding sequence. The promoter sequence can have transcription initiation function and comprise one or more synthetic motif sequences, wherein each synthetic motif sequence comprises a nucleic acid sequence that has at least 95% (95%, 96%, 97%, 98%, 99%, or 100%) sequence identity to any one of SEQ ID NOs: 1-3. Alternatively or additionally, the promoter sequence/molecule can comprise a Pisum sativum ubiquitin promoter (PsUBI promoter, e.g., PsUBI3p, PsUBI3-LIKEp, PsUBI10p) or a homolog of PsUBI promoters from other legumes such as Arachis hypogaea (peanut), Cicer arietinum (chickpea), Lupinus albus (white lupin), Lotus japonicus, Phaseolus acutifolius (tepary bean), Phaseolus lunatus (lima bean), Phaseolus vulgaris (common bean), Vigna unguiculata (Cowpea), e.g., AhUBI3p, AhUBI3-LIKEp, AhUBI4p, CaUBI3p, CaUBI4p, LaUBI3p, LaUBI4Ap, LaUBI4Bp, LaUBBp, LjUBI3p, LjUBI3-LIKEp, LjUBI4p, PaUBI3-LIKEp, PaUBI4p, PaUBBp, PlUBI3Ap, PlUBI3Bp, PlUBBp, PvUBI3p, PvUBI4p, PvUBBp, VuUBI3p, VuUBI4p, VuUBBp. For example, the promoter sequence/molecule can comprise a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-8 and 12-35, and retains transcription initiation function, or a nucleic acid sequence of any one of SEQ ID NOs: 6-8 and 12-35.

In some embodiments, the promoter molecule of the plant or plant part comprises at least two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3.

In some embodiments, the DNA construct comprises one or more linkers. In some embodiments, a linker connects the at least two synthetic motif sequences in the promoter. In some embodiments, a linker connects the promoter sequence and one of the synthetic motif sequence at the site of insertion. In some embodiments, a linker connects the promoter sequence and the coding sequence. In some embodiments, the linker has a nucleic acid sequence that is about 1-100, 1-90, 1-80, 1-70, 1-60, 1-50, 1-40, 1-30, 1-20, 1-10, 1-5, 5-10, 10-15, 15-20, 20-25, 25-30, 35-40, 40-45, 45-50, 50-55, 55-60, 60-65, 65-70, 70-75, 75-80, 80-85, 85-90, 90-95, 95-100, or more than 100 (e.g., about 1, 2, 3, 4, 5, 6, 7, 8, 9, 10, 15, 20, 25, 30, 35, 40, 45, 50, 55, 60, 65, 70, 75, 80, 85, 90, 95, 100, or more) nucleotides in length. In a specific embodiment, the linker is 5 nucleotides in length. In a further embodiment, the linker comprises a nucleic acid sequence of ACGTA or TTATG, or a nucleic acid sequence that shares at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with ACGTA or TTATG. In a specific embodiment, the promoter molecule comprises the linker ACGTA connecting the promoter sequence and the first motif sequence, and the linker TTATG connecting the first motif sequence and the second motif sequence, and the linker C connecting the second motif sequence and the promoter sequence.

In some embodiments, the promoter molecule of the plant or plant part comprises two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3. In some embodiments, said two synthetic motif sequences are different synthetic motif sequences.

In some embodiments, said two or at least two synthetic motif sequences comprise:
(a) a nucleic acid sequence that has at least 95% (95%, 96%, 97%, 98%, 99%, or 100%) sequence identity to SEQ ID NO: 1, or the nucleic acid sequence of SEQ ID NO: 1; and
(b) a nucleic acid sequence that has at least 95% (95%, 96%, 97%, 98%, 99%, or 100%) sequence identity to SEQ ID NO: 2; or the nucleic acid sequence of SEQ ID NO: 2.

In some embodiments, said one or more synthetic motif sequences are inserted into the 5′ UTR of said promoter sequence. In some embodiments, said one or more synthetic motif sequences are inserted into a 5′ intron, e.g., a mid-intron, of said promoter sequence.

In some embodiments, the promoter molecule of the plant or plant part comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 6, further comprising one or more copies of the one or more motif sequences inserted therein; or (ii) a nucleic acid sequence of SEQ ID NO: 6, further comprising one or more copies of the one or more motif sequences inserted therein. In some embodiments, the promoter molecule of the plant or plant part comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 9-11 and 79-81 or (ii) the nucleic acid sequence of any one of SEQ ID NOs: 9-11 and 79-81.

Promoter sequences disclosed herein as part of a plant or plant part can have mutations in at least 2, at least 3, at least 4, or all 5 of the following positions [[ ]]. In specific embodiments, the promoters disclosed herein as part of a plant or plant part can have at least 1, in at least 2, at least 3, at least 4, or all 5 of the following mutations: [[ ]].

In some embodiments, the coding sequence of the DNA construct of the plant or plant part encodes a guide RNA or a nuclease. In some embodiments, the coding sequence of the DNA construct encodes a guide RNA and a nuclease, both operably linked to the promoter molecule of the DNA construct. In some embodiments, the polynucleotide of the DNA construct operably linked to the promoter molecule encodes a guide RNA, and the DNA construct further comprises, in operable linkage, a nucleic acid molecule encoding a second promoter molecule and a polynucleotide encoding a nuclease. In some embodiments, the second promoter molecule is an RNA polymerase II promoter molecule. In some embodiments, the second promoter molecule has transcription initiation function and comprises: one or more synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; or (ii) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-43 and 79-81, and retains transcription initiation function, or a nucleic acid sequence of any one of SEQ ID NOs: 6-43 and 79-81.

In some embodiments, the coding sequence of the DNA construct operably linked to the promoter molecule encodes a nuclease, and the DNA construct further comprises, in operable linkage: a nucleic acid molecule encoding a second promoter molecule and a nucleic acid sequence encoding a guide RNA. In some embodiments, the second promoter molecule is an RNA polymerase III promoter molecule. In some embodiments, the second promoter molecule has transcription initiation function and comprises: one or more synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; or (ii) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-35, 43-77, and 79-81, and retains transcription initiation function, or a nucleic acid sequence of any one of SEQ ID NOs: 6-35, 43-77, and 79-81.

Where the plant or plant part comprises a DNA construct comprising coding sequences of a nuclease and a guide RNA, exemplary combinations of promoters for a nuclease and a guide RNA coding sequences are indicated with “X” in Table 1. Each promoter listed is meant to include its variants, e.g., promoter molecules that share at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) similarity with the promoter molecule indicated.

In some specific embodiments, the plant or plant part of the present disclosure comprises:
PsUBI3-SYN3p operably linked to a polynucleotide encoding a nuclease and a gRNA;
PsUBI3-SYN3p operably linked to a polynucleotide encoding a nuclease and AtU6-26p operably linked to a polynucleotide encoding a gRNA;
PsUBI3-SYN3p operably linked to a polynucleotide encoding a nuclease and PsUBI3p operably linked to a polynucleotide encoding a gRNA;
PsUBI3-SYN3p operably linked to a polynucleotide encoding a nuclease and AtUBI11p operably linked to a polynucleotide encoding a gRNA;
PsUBI3-SYN27p operably linked to a polynucleotide encoding a nuclease and a gRNA;
PsUBI3-SYN27p operably linked to a polynucleotide encoding a nuclease and AtU6-26p operably linked to a polynucleotide encoding a gRNA;
PsUBI3-SYN27p operably linked to a polynucleotide encoding a nuclease and PsUBI27p operably linked to a polynucleotide encoding a gRNA;
PsUBI3-SYN27p operably linked to a polynucleotide encoding a nuclease and AtUBI11p operably linked to a polynucleotide encoding a gRNA;
PsUBI3 operably linked to a polynucleotide encoding a nuclease and PsUBI3-SYN3p operably linked to a polynucleotide encoding a gRNA;
PsUBI3p operably linked to a polynucleotide encoding a nuclease and a gRNA;
PsUBI3p operably linked to a polynucleotide encoding a nuclease, and AtUBI11p operably linked to a polynucleotide encoding a gRNA;
AtUBI11p operably linked to a polynucleotide encoding a nuclease, and PsUBI3-SYN3p operably linked to a polynucleotide encoding a gRNA; or
AtUBI11p operably linked to a polynucleotide encoding a nuclease, and PsUBI3p operably linked to a polynucleotide encoding a gRNA.

In some embodiments, the nuclease encoded by the polynucleotide of the DNA construct is a CRISPR-associated Cas endonuclease. In specific embodiments, the CRISPR nuclease is a Cas12a (Cpf1) nuclease. In a specific embodiment, the Cas12a nuclease is a McCpf1 nuclease, e.g., a Mc.2Cpf1 2C-NLS nuclease. In some embodiments, the CRISPR nuclease shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 78 or comprises the sequence of SEQ ID NO: 78. In some embodiments, the nuclease is further operably linked to one or more nuclear localization sequences (NLSs) and/or one or more epitope tags.

In some embodiments, the DNA construct of the present disclosure further comprises, in operable linkage, a polynucleotide encoding a selectable marker and/or a regulatory RNA. The selectable marker and/or the regulatory RNA can be operably linked to the promoter of the present disclosure, which can be the same promoter as the one to which said coding sequence (e.g., for a guide RNA or a nuclease) is operably linked, or a different promoter, or any other promoter. In some embodiments, the regulatory RNA is operably linked to a promoter molecule comprising: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) identity to any one of SEQ ID NOs: 6-35, 43-77 and 79-81, and retains transcription initiation function; or (ii) a nucleic acid sequence of any one of SEQ ID NOs: 6-35, 43-77, and 79-81.

The coding sequence(s) of the DNA construct can encode any polynucleotide of interest, for expression driven by the promoter molecule of the present disclosure. For example, the coding sequence(s) may encode editing reagents (e.g., a guide RNA, a nuclease) targeting any gene or genomic site of interest, regulatory RNA, a selectable marker/reporter, an enzyme, a transcription factor, a receptor, or a ligand.

### A. Plants Comprising Increased Expression or Function of Polynucleotide of Interest Operably Linked to the Promoter

In some embodiments, the plants or plant parts of the present disclosure can have increased expression or function (e.g., editing efficiency) of one or more molecules encoded by the polynucleotide(s) of interest (e.g., editing reagents) operably linked to the promoter molecules in the plant or plant part, relative to a control plant or plant part, wherein the control plant or plant part comprises at least one of the polynucleotide(s) of interest operably linked to a control promoter. The control promoter, as used herein, does not comprise any one of: one or more synthetic motif sequences each comprising a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-35 and 79-81; or a nucleic acid sequence of any one of SEQ ID NOs: 6-35 and 79-81.

Expression or function of polynucleotide(s) of interest operably linked to the promoter molecule can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 4%5% 50%, 55%, 60, 65%, 70%, 75%, 80%, 85%, 90%, 95%, 100%, 200%, 300%, 400%, 500%, 600%, 700%, 800%, 900%, 1000%, or more in the plant or plant part comprising the promoter molecule, relative to a control plant or plant part comprising the polynucleotide of interest operably linked to a control promoter.

Expression or function of an operably linked polynucleotides of interest can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by in the plant or plant part comprising the promoter molecule compared to a control plant or plant part comprising the polynucleotides of interest operably linked to a control promoter. Polynucleotide and gene expression levels can be measured by any methods known in the art. For example, expression levels can be measured by quantifying levels of the polynucleotide or gene product, e.g., an RNA or a protein, by, e.g., PCR, real-time PCR, Western blotting, and ELISA. Polynucleotide or gene expression levels can also be assessed by quantifying levels of function of polynucleotide or gene product, for example by quantifying the occurrence of events caused by the polynucleotide or gene product (e.g., editing frequency of a target gene) or by quantifying the levels of product produced by the polynucleotide or gene product.

Plants or plant parts comprising a promoter having insertion of one or more of the synthetic motif sequences can have increased expression or function (e.g., editing frequency) of operably linked polynucleotide(s) of interest (e.g., editing reagents) relative to a control plant or plant part comprising a control promoter without such insertion. For instance, plants or plant parts comprising a promoter having insertion of one copy of the synthetic motif sequence can have increased expression or function of operably linked polynucleotide(s) of interest relative to a control plant or plant part comprising a control promoter without such insertion. Further, plants or plant parts comprising a promoter having insertion of more than one copies of the synthetic motif sequences can have increased expression or function of operably linked polynucleotide(s) of interest relative to a control plant or plant part comprising a control promoter comprising one or no copy of the synthetic motif sequence. Still further, plants or plant parts comprising a promoter having insertion of one or more copies of at least two different motif sequences into a promoter molecule can have increased expression or function of an operably linked polynucleotide of interest relative to a control plant or plant part comprising a promoter comprising the one or more copies of: only one of the at least two motif sequences; or more than one but less than all of the at least two motif sequences.

For instance, a plant or plant part comprising a promoter having insertion of one copy of a synthetic motif, e.g., the synthetic motif sequence from PsUBI3-LIKEp (SEQ ID NO: 1) or a variant thereof or the synthetic motif sequence from PsUBI10p (SEQ ID NO: 2) or a variant thereof, e.g., a promoter such as PsUBI3-SYN1p (SEQ ID NO: 9) or PsUBI3-SYN2p (SEQ ID NO: 10), can have increased expression or function of an operably linked polynucleotide of interest relative to a plant or plant part comprising a promoter molecule (e.g., PsUBI3p, SEQ ID NO: 6) comprising no synthetic motif sequence. Further, a plant or plant part comprising a promoter having insertion of one copy of the synthetic motif sequence from PsUBI3-LIKEp (SEQ ID NO: 1) or a variant thereof and one copy of the synthetic motif sequence from PsUBI10p (SEQ ID NO: 2) or a variant thereof, e.g., the synthetic motif sequence set forth as SEQ ID NO: 3 or a variant thereof, e.g., a promoter such as PsUBI3-SYN3p (SEQ ID NO: 11), can have increased expression or function of an operably linked polynucleotide of interest relative to not only a plant or plant part comprising a promoter molecule (e.g., PsUBI3p, SEQ ID NO: 6) comprising no synthetic motif sequence but also a plant or plant part comprising a promoter molecule (e.g., PsUBI3-SYN1p, set forth as SEQ ID NO: 9, or PsUBI3-SYN2p, set forth as SEQ ID NO: 10) comprising a copy of only one of the synthetic motif sequence. Further, a plant or plant part comprising a promoter having insertion of more than one copy of the synthetic motif sequence from PsUBI3-LIKEp (SEQ ID NO: 1) or a variant thereof and/or the synthetic motif sequence from PsUBI10p (SEQ ID NO: 2) or a variant thereof, with at least one copy of each of the two synthetic motif sequences, can have increased expression or function of an operably linked polynucleotide of interest relative to a plant or plant part comprising a promoter molecule with one copy of each of the two motif sequences. The increase of expression or function of an operably linked polynucleotide of interest in the plant or plant part comprising a promoter described hereinabove can be by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%00, 200%, 300%, 400%, 500%, 600%, 700%, 800%, 900%, 1000%, or more, as compared to a control plant or plant part comprising a control promoter. Expression or function of an operably linked polynucleotide of interest can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more. Gene or polynucleotide expression levels can be measured by any methods known in the art. For example, gene or polynucleotide expression levels can be measured by quantifying levels of the gene product, e.g., an RNA or a protein, by, e.g., PCR, real-time PCR, Western blotting, and ELISA. Gene or polynucleotide expression levels can also be assessed by quantifying levels of function of gene or polynucleotide product, for example by quantifying the occurrence of events caused by the gene or polynucleotide product (e.g., editing frequency of a target gene) or by quantifying the levels of product produced by the gene or polynucleotide product.

### B. Plants Comprising Increased Editing Frequency at Target Site

In some embodiments, plants of the present disclosure comprise editing reagents such as a guide RNA and a nuclease operably linked to the promoter of the present disclosure. Editing reagents targeting any gene or genomic site of interest in a plant or plant parts can be used, i.e., operably linked to a promoter of the present disclosure and introduced into a plant or plant part. In such embodiments, the plants or plant parts of the present disclosure can have increased expression or function of editing reagents (e.g., a guide RNA and/or a nuclease), relative to a control plant or plant part, which comprises the editing reagents (the guide RNA and/or the nuclease) with at least one of which operably linked to a control promoter. Accordingly, in certain embodiments, plants or plant parts of the present disclosure can have an increased frequency or number of mutations introduced into the genome at the target site of the editing reagents. Editing frequency or the number of mutations can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more in the plant or plant part, as compared to a control plant or plant part comprising a control promoter. Editing frequency or the number of mutations can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more in the plant or plant part compared to a control plant or plant part comprising a control promoter. In specific embodiments, the plant or plant part of the present disclosure can have an increased frequency or number of mutations at a target site of the editing reagents by about 10% to about 500%, resulting in about 1.1-fold to about 6-fold increase, relative to a control plant or plant part comprising a control promoter.

### C. Other Characteristics

In some embodiments, the promoter molecule(s) and/or the polynucleotide(s) of interest are stably inserted in the genome of said plant or plant part. In particular embodiments, the promoter molecule(s) and/or the polynucleotide(s) of interest are transiently expressed in the plant or plant part and/or are not integrated into the plant genome.

In some embodiments, the polynucleotide of interest is constitutively expressed in the plant or plant part. In some embodiments, the polynucleotide of interest is expressed throughout (i.e., ubiquitous expression) the plant tissues and cells. In some embodiments, the polynucleotide of interest is expressed more strongly in certain tissues or cells, e.g., meristematic tissues or cells, compared to other tissues or cells. In some embodiments, the polynucleotide of interest is expressed in a developmentally-regulated manner. In some embodiments, the polynucleotide of interest is expressed upon induction via an inducible promoter of the present disclosure.

A plant or plant part of the present disclosure can be a monocot. Alternatively, a plant or plant part of the present disclosure can be a dicot. A plant or plant part of the present disclosure can be a crop plant or part of a crop plant. Examples of crop plants include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, or alternatively, a plant or plant part of the present disclosure can be a legume, i.e., a plant belonging to the family Fabaceae (or Leguminosae), or a part (e.g., fruit or seed) of such a plant. When used as a dry grain, the seed of a legume is also called a pulse. Examples of legume include, without limitation, beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Vicia faba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally, or alternatively, a plant or plant part of the present disclosure can be an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. Aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). For example, a plant or plant part of the present disclosure can be Pisum sativum or a part of Pisum sativum.

### D. Plant Parts, Plant Cells, and Plant Products

Also provided herein are plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), plant extract (e.g., protein, sweetener, antioxidants, alkaloids, etc.), plant concentrate (e.g., whole plant concentrate, plant part concentrate, or protein concentrate), plant powder [e.g., formulated powder, such as formulated plant part powder (e.g., seed flour)], and plant biomass (e.g., dried biomass, such as crushed and/or powdered biomass) obtained from plants of the present disclosure. Also provided herein are seeds, such as a representative sample of seeds, from a plant of the present disclosure.

Molecules encoded by the DNA constructs of the present disclosure (e.g., promoters, editing reagents) may be found in plants or plant parts to which the DNA constructs have been introduced, or plants or plant parts regenerated therefrom according to the methods of the present disclosure. Mutations introduced by the methods using the DNA constructs encoding editing reagents may be found in plants or plant parts to which the DNA constructs have been introduced, or plants or plant parts regenerated therefrom according to the methods of the present disclosure. Mutations can also be found in plant parts, plant extract, plant concentrate, plant powder, and plant biomass obtained from such plants.

Also provided herein are food and/or beverage products containing plant compositions (e.g., plant parts, plant extract, plant concentrate, plant powder, plant protein, and plant biomass) described hereinabove, such as plant compositions derived from the plants or plant parts of the present disclosure. Such food and/or beverage products include, without limitation, shakes, juices, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc, and condiments. A food and/or beverage product that contains plant compositions obtained from plants or plant parts of the present disclosure can have desired traits, compared to a similar or comparable food and/or beverage product that contains plant compositions obtained from a control plant or plant part.

While the invention is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, grains, leaves, flowers, branches, fruit, kernels, ears, cobs, husks, stalks, roots, root tips, anthers, and the like. Progeny, variants, and mutants of the regenerated plants are also included within the scope of the invention, provided that these parts comprise the introduced polynucleotides.

### VI. Methods of Expressing a Polynucleotide of Interest in a Plant

Disclosed herein are methods of expressing a nucleotide sequence of interest in a plant or plant part (e.g., juice, pulp, seed, fruit, flower, nectar, embryo, pollen, ovule, leaf, stem, branch, bark, kernel, ear, cob, husk, stalk, root, root tip, anther) by introducing into the plant or the plant part the promoter molecule or the DNA construct of the present disclosure. In some embodiments, the promoter molecule or the DNA construct is introduced into the plant or the plant part by stable transformation. In other embodiments, the promoter molecule or the DNA construct is introduced into the plant by transient transformation.

In some aspects, the present disclosure provides methods of expressing a polynucleotide of interest in a plant or plant part comprising introducing a DNA construct into the plant or plant part, wherein the DNA construct comprises, in operable linkage: (a) a nucleic acid molecule comprising a promoter sequence and (b) a polynucleotide of interest. The promoter sequence can have transcription initiation function and comprise one or more synthetic motif sequences, wherein each synthetic motif sequence comprises: a nucleic acid sequence that has at least 95% (95%, 96%, 97%, 98%, 99%, or 100%) sequence identity to any one of SEQ ID NOs: 1-3; or the nucleic acid sequence of any one of SEQ ID NOs: 1-3. Alternatively or additionally, the promoter sequence/molecule can comprise a Pisum sativum ubiquitin promoter (PsUBI promoter, e.g., PsUBI3p, PsUBI3-LIKEp, PsUBI10p) or a homolog of PsUBI promoters from other legumes such as Arachis hypogaea (peanut), Cicer arietinum (chickpea), Lupinus albus (white lupin), Lotus japonicus, Phaseolus acutifolius (tepary bean), Phaseolus lunatus (lima bean), Phaseolus vulgaris (common bean), Vigna unguiculata (cowpea), e.g., AhUBI3p, AhUBI3-LIKEp, AhUBI4p, CaUBI3p, CaUBI4p, LaUBI3p, LaUBI4Ap, LaUBI4Bp, LaUBBp, LjUBI3p, LjUBI3-LIKEp, LjUBI4p, PaUBI3-LIKEp, PaUBI4p, PaUBBp, PlUBI3Ap, PlUBI3Bp, PlUBBp, PvUBI3p, PvUBI4p, PvUBBp, VuUBI3p, VuUBI4p, VuUBBp. For example, the promoter sequence/molecule can comprise a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-8 and 12-35, and retains transcription initiation function, or a nucleic acid sequence of any one of SEQ ID NOs: 6-8 and 12-35.

In some aspects, the present disclosure provides methods of expressing a polynucleotide of interest in a plant or plant part comprising: (i) introducing a DNA construct into a plant cell, wherein the DNA construct comprises, in operable linkage (a) a nucleic acid molecule comprising a promoter sequence and (b) a polynucleotide of interest; and (ii) regenerating a plant or plant part from said plant cell. The promoter sequence can have transcription initiation function and comprise one or more synthetic motif sequences, wherein each synthetic motif sequence comprises: a nucleic acid sequence that has at least 95% (95%, 96%, 97%, 98%, 99%, or 100%) sequence identity to any one of SEQ ID NOs: 1-3; or the nucleic acid sequence of any one of SEQ ID NOs: 1-3. Alternatively or additionally, the promoter sequence/molecule can comprise a Pisum sativum ubiquitin promoter (PsUBI promoter, e.g., PsUBI3p, PsUBI3-LIKEp, PsUBI10p) or a homolog of PsUBI promoters from other legumes such as Arachis hypogaea (peanut), Cicer arietinum (chickpea), Lupinus albus (white lupin), Lotus japonicus, Phaseolus acutifolius (tepary bean), Phaseolus lunatus (lima bean), Phaseolus vulgaris (common bean), Vigna unguiculata (cowpea), e.g., AhUBI3p, AhUBI3-LIKEp, AhUBI4p, CaUBI3p, CaUBI4p, LaUBI3p, LaUBI4Ap, LaUBI4Bp, LaUBBp, LjUBI3p, LjUBI3-LIKEp, LjUBI4p, PaUBI3-LIKEp, PaUBI4p, PaUBBp, PlUBI3Ap, PlUBI3Bp, PlUBBp, PvUBI3p, PvUBI4p, PvUBBp, VuUBI3p, VuUBI4p, VuUBBp. For example, the promoter sequence/molecule can comprise a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-8 and 12-35, and retains transcription initiation function, or a nucleic acid sequence of any one of SEQ ID NOs: 6-8 and 12-35.

In some embodiments, the promoter molecule of the methods of the present disclosure comprises at least two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3.

The DNA construct can comprise one or more linkers. In some embodiments, a linker connects the at least two synthetic motif sequences in the promoter. In some embodiments, a linker connects the promoter sequence and one of the synthetic motif sequence at the site of insertion. In some embodiments, a linker connects the promoter molecule and the polynucleotide of interest. In some embodiments, the linker has a nucleic acid sequence that is about 1-100, 1-90, 1-80, 1-70, 1-60, 1-50, 1-40, 1-30, 1-20, 1-10, 1-5, 5-10, 10-15, 15-20, 20-25, 25-30, 35-40, 40-45, 45-50, 50-55, 55-60, 60-65, 65-70, 70-75, 75-80, 80-85, 85-90, 90-95, 95-100, or more than 100 (e.g., about 1, 2, 3, 4, 5, 6, 7, 8, 9, 10, 15, 20, 25, 30, 35, 40, 45, 50, 55, 60, 65, 70, 75, 80, 85, 90, 95, 100, or more) nucleotides in length. In a specific embodiment, the linker is 5 nucleotides in length. In a further embodiment, the linker comprises a nucleic acid sequence of ACGTA or TTATG, or a nucleic acid sequence that shares at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with ACGTA or TTATG. In a specific embodiment, the promoter molecule comprises the linker ACGTA connecting the promoter sequence and the first motif sequence, and the linker TTATG connecting the first motif sequence and the second motif sequence, and the linker C connecting the second motif sequence and the promoter sequence.

In some embodiments, the promoter molecule of the methods comprises two synthetic motif sequences, wherein each synthetic motif sequence comprises: (i) a nucleic acid sequence that has at least 95% (95%, 96%, 97%, 98%, 99%, or 100%) sequence identity to any one of SEQ ID NOs: 1-3; or (ii) the nucleic acid sequence of any one of SEQ ID NOs: 1-3. In some aspects, said two synthetic motif sequences are different synthetic motif sequences.

In some embodiments, at least one of said synthetic motif sequences comprise a nucleic acid sequence that has at least 95% (95%, 96%, 97%, 98%, 99%, or 100%) sequence identity to SEQ ID NO: 1, or the nucleic acid sequence of SEQ ID NO: 1; and at least one of said synthetic motif sequences comprise a nucleic acid sequence that has at least 95% (95%, 96%, 97%, 98%, 99%, or 100%) sequence identity to SEQ ID NO: 2; or the nucleic acid sequence of SEQ ID NO: 2.

In some embodiments, said one or more synthetic motif sequences are inserted into the 5′ UTR of said promoter sequence. In some embodiments, said one or more synthetic motif sequences are inserted into a 5′ intron, e.g., a mid-intron region, of said promoter sequence.

In some embodiments, the promoter molecule of the methods of the present disclosure comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 6, further comprising one or more copies of the one or more synthetic motif sequences inserted therein; or (ii) the nucleic acid sequence of SEQ ID NO: 6, further comprising one or more copies of the one or more synthetic motif sequences inserted therein. In some embodiments, the promoter molecule comprises: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 9-11 and 79-81 or (ii) the nucleic acid sequence of any one of SEQ ID NOs: 9-11 and 79-81.

In some embodiments according to the methods of the present disclosure, the polynucleotide of interest of the DNA construct encodes a guide RNA or a nuclease. In some embodiments, the polynucleotide of interest of the DNA construct encodes a guide RNA and a nuclease, both operably linked to the promoter molecule of the DNA construct.

In some embodiments according to the methods of the present disclosure, the polynucleotide of interest of the DNA construct operably linked to the promoter molecule encodes a guide RNA, and the DNA construct further comprises, in operable linkage, a nucleic acid molecule encoding a second promoter molecule and a polynucleotide encoding a nuclease. In some embodiments, the second promoter molecule is an RNA polymerase II promoter molecule. In some embodiments, the second promoter molecule has transcription initiation function and comprises (a) one or more synthetic motif sequences comprising a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; or (b) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-43 and 79-81, and retains transcription initiation function, or a nucleic acid sequence of any one of SEQ ID NOs: 6-43 and 79-81.

In some embodiments according to the methods of the present disclosure, the polynucleotide of interest of the DNA construct operably linked to the promoter molecule encodes a nuclease, and the DNA construct further comprises, in operable linkage: a nucleic acid molecule encoding a second promoter molecule and a polynucleotide encoding a guide RNA. In some embodiments, the second promoter molecule is an RNA polymerase III promoter molecule. In some embodiments, the second promoter molecule has transcription initiation function and comprises (i) one or more synthetic motif sequences, wherein each synthetic motif sequence comprises a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; or (ii) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-35, 43-77, and 79, and retains transcription initiation function, or a nucleic acid sequence of any one of SEQ ID NOs: 6-35, 43-77, and 79-81.

Where the method comprises introducing a plant cell a DNA construct comprising polynucleotides encoding a nuclease and a guide RNA, exemplary combinations of promoters for polynucleotides encoding a nuclease and a guide RNA are indicated with “X” in Table 1. Each promoter listed is meant to include its variants, e.g., promoter sequences that share at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) similarity with the promoter molecule indicated.

In some specific embodiments, the present disclosure provides a method of expressing a polynucleotide of interest in a plant or plant part, wherein the polynucleotide of interest encodes a nuclease and a guide RNA, and wherein the method comprises:
(i) introducing into a plant, plant part, or plant cell a DNA construct comprising:
   PsUBI3-SYN3p operably linked to a polynucleotide encoding a nuclease and a gRNA;
   PsUBI3-SYN3p operably linked to a polynucleotide encoding a nuclease and AtU6-26p operably linked to a polynucleotide encoding a gRNA;
   PsUBI3-SYN3p operably linked to a polynucleotide encoding a nuclease and PsUBI3p operably linked to a polynucleotide encoding a gRNA;
   PsUBI3-SYN3p operably linked to a polynucleotide encoding a nuclease and AtUBI11p operably linked to a polynucleotide encoding a gRNA;
   PsUBI3 operably linked to a polynucleotide encoding a nuclease and PsUBI3-SYN3p operably linked to a polynucleotide encoding a gRNA;
   PsUBI3p operably linked to a polynucleotide encoding a nuclease and a gRNA;
   PsUBI3p operably linked to a polynucleotide encoding a nuclease, and AtUBI11p operably linked to a polynucleotide encoding a gRNA;
   AtUBI11p operably linked to a polynucleotide encoding a nuclease, and PsUBI3-SYN3p operably linked to a polynucleotide encoding a gRNA; or
   AtUBI11p operably linked to a polynucleotide encoding a nuclease, and PsUBI3p operably linked to a polynucleotide encoding a gRNA; and
(ii) optionally regenerating a plant or plant part from said plant, plant part, or plant cell.

In some embodiments, the nuclease encoded by the polynucleotide of interest of the DNA construct is a CRISPR-associated Cas endonuclease. In specific embodiments, the CRISPR nuclease is a Cas12a (Cpf1) nuclease. In a specific embodiment, the Cas12a nuclease is a McCpf1 nuclease, e.g., a Mc.2Cpf1 2C-NLS nuclease. In some embodiments, the CRISPR nuclease shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 78 or comprises the sequence of SEQ ID NO: 78. In some embodiments, the nuclease is further operably linked to one or more nuclear localization sequences (NLSs) and/or one or more epitope tags.

In some embodiments according to the methods of the present disclosure, the DNA construct further comprises, in operable linkage, a nucleic acid sequence encoding a selectable marker and/or a regulatory RNA. In some embodiments, wherein the DNA construct further comprises a promoter molecule operably linked to the regulatory RNA and comprising: (i) a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) identity to any one of SEQ ID NOs: 6-35, 43-77, and 79-81, and retains transcription initiation function; or (ii) a nucleic acid sequence of any one of SEQ ID NOs: 6-35, 43-77, and 79-81.

In some embodiments according to the methods of the present disclosure, the polynucleotide of interest is stably inserted into the genome of the plant or plant part. In some embodiments, the plant or the plant part is stably transformed. Alternatively, the polynucleotide of interest can be transiently expressed in the plant or plant part.

In some embodiments, the polynucleotide of interest is constitutively expressed in the plant or plant part. In some embodiments, the polynucleotide of interest is expressed throughout the plant tissues and cells. In some embodiments, the polynucleotide of interest is expressed more strongly in certain tissues or cells, e.g., meristematic tissues or cells, compared to other tissues or cells. In some embodiments, the polynucleotide of interest is expressed in a developmentally-regulated manner. In some embodiments, the polynucleotide of interest is expressed upon induction via an inducible promoter of the present disclosure.

### A. Increasing Expression or Function of a Polynucleotide of Interest Operably Linked to the Promoter

In some embodiments, the methods of the present disclosure increase expression or function (e.g., editing efficiency) of one or more molecules encoded by the polynucleotide(s) of interest and/or the polynucleotide(s) of interest (e.g., editing reagents) operably linked to the promoter molecules in the plant or plant part, relative to a control plant or plant part, wherein the control plant or plant part comprises the polynucleotide of interest and/or at least one of the polynucleotide(s) operably linked to a control promoter. The control promoter, as used herein, does not comprise any one of: one or more synthetic motif sequences each comprising a nucleic acid sequence that has at least 95% (95%, 96%, 97%, 98%, 99%, or 100%) sequence identity to any one of SEQ ID NOs: 1-3 or the nucleic acid sequence of any one of SEQ ID NOs: 1-3; a nucleic acid sequence that has at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 6-35 and 79-81; or a nucleic acid sequence of any one of SEQ ID NOs: 6-35 and 79-81.

Expression or function of polynucleotide(s) of interest can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%00, 100%, 200%, 300%, 400%, 500%, 600%, 700%, 800%, 900%, 1000%, or more with methods using a promoter of the present disclosure, as compared to using a control promoter. Expression or function of an operably linked polynucleotide of interest can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by using a promoter of the present disclosure compared to a control promoter. In specific embodiments the polynucleotides of interest are genes encoding a protein. Gene or polynucleotide expression levels can be measured by any methods known in the art. For example, gene or polynucleotide expression levels can be measured by quantifying levels of the gene or polynucleotide product, e.g., an RNA or a protein, by, e.g., PCR, real-time PCR, Western blotting, and ELISA. Gene or polynucleotide expression levels can also be assessed by quantifying levels of function of gene or polynucleotide product, for example by quantifying the occurrence of events caused by the gene or polynucleotide product (e.g., editing frequency of a target gene) or by quantifying the levels of product produced by the gene or polynucleotide product.

The methods of the present disclosure can be used to express or enhance expression of any polynucleotide of interest, and are not limited to exemplary polynucleotides of interest described herein. For example, the polynucleotide(s) may encode editing reagents (e.g., a guide RNA, a nuclease), regulatory RNA, a selectable marker/reporter, an enzyme, a transcription factor, a receptor, or a ligand, for expression or enhanced expression in a plant or plant part, according to the methods of the present disclosure. Editing reagents targeting any gene or genomic site of interest in a plant or plant parts can be used according to the methods of the present disclosure.

Using a promoter having insertion of one or more synthetic motif sequences according to the methods of the present disclosure can increase expression or function (e.g., editing frequency) of operably linked polynucleotide(s) of interest (e.g., editing reagents) relative to a control promoter without such insertion. For instance, using a promoter having insertion of one copy of the synthetic motif sequence can increase expression or function of operably linked polynucleotide(s) relative to a control promoter without such insertion. Further, using a promoter having insertion of more than one copies of the synthetic motif sequences can increase expression or function of operably linked polynucleotide(s) relative to a control promoter comprising one or no copy of the synthetic motif sequence. Still further, a promoter having insertion of one or more copies of at least two different motif sequences into a promoter molecule can increase expression or function of an operably linked polynucleotide relative to a promoter comprising the one or more copies of: only one of the at least two motif sequences; or more than one but less than all of the at least two motif sequences.

For instance, using a promoter having insertion of one copy of a synthetic motif, e.g., the synthetic motif sequence from PsUBI3-LIKEp (SEQ ID NO: 1) or a variant thereof or the synthetic motif sequence from PsUBI10p (SEQ ID NO: 2) or a variant thereof, e.g., a promoter such as PsUBI3-SYN1p (SEQ TD NO: 9) or PsUBI3-SYN2p (SEQ TD NO: 10), can increase expression or function (e.g., editing efficiency) of an operably linked polynucleotide (e.g., an editing reagent) relative to a promoter molecule (e.g., PsUBI3p, SEQ ID NO: 6) comprising no synthetic motif sequence. Further, using a promoter having insertion of one copy of the synthetic motif sequence from PsUBI3-LIKEp (SEQ ID NO: 1) or a variant thereof and one copy of the synthetic motif sequence from PsUBI10p (SEQ ID NO: 2) or a variant thereof, e.g., the synthetic motif sequence set forth as SEQ ID NO: 3 or a variant thereof, e.g., a promoter such as PsUBI3-SYN3p (SEQ ID NO: 11), can increase expression or function (e.g., editing efficiency) of an operably linked polynucleotide (e.g., an editing reagent) relative to not only a promoter molecule (e.g., PsUBI3p, SEQ ID NO: 6) comprising no synthetic motif sequence but also a promoter molecule (e.g., PsUBI3-SYN1p, set forth as SEQ ID NO: 9, or PsUBI3-SYN2p, set forth as SEQ ID NO: 10) comprising a copy of only one of the synthetic motif sequence. Further, using a promoter having insertion of more than one copy of the synthetic motif sequence from PsUBI3-LIKEp (SEQ ID NO: 1) or a variant thereof and/or the synthetic motif sequence from PsUBI10p (SEQ ID NO: 2) or a variant thereof, with at least one copy of each of the two synthetic motif sequences, can increase expression or function (e.g., editing efficiency) of an operably linked polynucleotide (e.g., an editing reagent) relative to a promoter molecule with one copy of each of the two motif sequences. The increase of expression or function of an operably linked polynucleotide by using a promoter described hereinabove can be by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more with methods using a promoter of the present disclosure, as compared to using a control promoter. Expression or function of an operably linked polynucleotide of interest can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more. Gene or polynucleotide expression levels can be measured by any methods known in the art. For example, gene expression levels can be measured by quantifying levels of the gene or polynucleotide product, e.g., an RNA or a protein, by, e.g., PCR, real-time PCR, Western blotting, and ELISA. Gene or polynucleotide expression levels can also be assessed by quantifying levels of function of gene or polynucleotide product, for example by quantifying the occurrence of events caused by the gene or polynucleotide product (e.g., editing frequency of a target gene) or by quantifying the levels of product produced by the gene or polynucleotide product.

The synthetic motif sequence can be a sequence that is not present in the promoter sequence to which the synthetic motif sequence is inserted. The synthetic motif sequence can be a sequence that shares less than 80% similarity with any part of the promoter sequence to which the synthetic motif sequence is inserted. Alternatively, the synthetic motif sequence can be a sequence that shares at least 80% (80%, 81%, 82%, 83%, 84%, 85%, 86%, 87%, 88%, 89%, 90%, 91%, 92%, 93%, 94%, 95%, 96%, 97%, 98%, 99%, or 100%) sequence similarity with, or identical to, part of the promoter sequence to which the synthetic motif sequence is inserted.

### B. Increasing Editing Efficiency at a Target Site

In some embodiments, methods of the present disclosure can be used to express editing reagents such as a guide RNA or a nuclease in a plant or plant part that are useful for introducing a mutation at a target site in the genome of a plant. Editing reagents targeting any gene or genomic site of interest in a plant or plant parts can be used, i.e., operably linked to a promoter of the present disclosure and introduced into a plant or plant part, according to the methods of the present disclosure. In such embodiments, the methods of the present disclosure can increase expression or function of editing reagents (e.g., a guide RNA and/or a nuclease) in the plant or plant part, relative to a control plant or plant part, wherein the control plant or plant part comprises the editing reagents (the guide RNA and/or the nuclease) and at least one of them are operably linked to a control promoter. Accordingly, in certain embodiments, the methods of the present disclosure can increase the efficiency or frequency of introducing a mutation to a genome of a plant or plant part, e.g., editing frequency at a target site. Editing frequency can be increased by about 10-100%, 20-10⁰%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by methods using a promoter of the present disclosure, as compared to a control plant or plant part comprising a control promoter. Editing frequency can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by methods using a promoter of the present disclosure compared to a control promoter. In specific embodiments, the method of the present disclosure can increase an efficiency of introducing a mutation to a genome of a plant or plant part, e.g., editing frequency at a target site of the editing reagents, is by about 10% to about 500% in the plant or plant part, resulting in about 1.1-fold to about 6-fold increase, relative to a control plant or plant part comprising a control promoter.

### C. Introducing Mutations to Plants

In some embodiments, methods and compositions of the present disclosure can be used to introduce mutations in the genome of a plant. Editing reagents targeting any gene or genomic site of interest in a plant or plant parts can be expressed from the promoters disclosed herein. Further, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g. using a gene-gun), Agrobacterium infection and/or infection by other bacterial species capable of transferring DNA into plants (e.g., Ochrobactrum spp., Ensifer spp., Rhizobium spp.), viral infection, and other techniques can be used to deliver nucleic acid sequences into a plant described herein. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

Similarly, methods disclosed herein are not limited to certain techniques of mutagenesis. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein. Introducing mutations into plants or plant parts to obtain desired traits may be achieved through the use of precise genome-editing technologies to modulate the expression of the endogenous sequence. In this manner, a nucleic acid sequence can be inserted, substituted, or deleted proximal to or within a native plant sequence encoding a polynucleotide of interest through the use of methods available in the art. Such methods include, but are not limited to, use of meganucleases designed against the plant genomic sequence of interest (D'Halluin et al (2013) Plant Biotechnol J 11: 933-941); CRISPR-Cas9, CRISPR-Cas12a (Cpf1), transcription activator-like effector nucleases (TALENs), zinc finger nucleases (ZFNs), and other technologies for precise editing of genomes [Feng et al. (2013) Cell Research 23:1229-1232, Podevin et al. (2013) Trends Biotechnology 31: 375-383, Wei et al. (2013) J Gen Genomics 40:281-289, Zhang et al (2013) WO 2013/026740, Zetsche et al. (2015) Cell 163:759-771, U.S. Provisional Patent Application 62/295,325]; N. gregoryi Argonaute-mediated DNA insertion (Gao et al. (2016) Nat Biotechnol doi:10.1038/nbt.3547); Cre-lox site-specific recombination (Dale et al. (1995) Plant J 7:649-659; Lyznik, et al. (2007) Transgenic Plant J 1:1-9; FLP-FRT recombination (Li et al. (2009) Plant Physiol 151:1087-1095); Bxb1-mediated integration (Yau et al. (2011) Plant J 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol 701: 51-65; Puchta (2002) Plant Mol Biol 48:173-182).

In some aspects, inserting, substituting, or deleting one or more nucleotides at a precise location of interest may be achieved using a meganuclease or other suitable nuclease system designed to target the genomic sequence of interest. Without wishing to be bound by theory, a nuclease system can be used to achieve insertion, substitution, or deletion of genetic elements at a predefined genomic locus by causing a double-strand break at said predefined genomic locus and, optionally, providing an appropriate DNA template for insertion. This strategy is well-understood and has been demonstrated previously to insert a transgene at a predefined location in the cotton genome (D'Halluin et al. (2013) Plant Biotechnol J 11: 933-941). For example, a Cas12a (Cpf1) endonuclease coupled with a guide RNA (guide RNA) designed against the genomic sequence of interest can be used (i.e., a CRISPR-Cas12a system). Alternatively, a Cas9 endonuclease coupled with a guide RNA designed against the genomic sequence of interest (a CRISPR-Cas9 system), or a Cms1 endonuclease coupled with a guide RNA designed against the genomic sequence of interest (a CRISPR-Cms1) can be used. Other nuclease systems for use with the methods of the present invention include CRISPR systems (e.g., Type I, Type II, Type III, Type IV, and/or Type V CRISPR systems (Makarova et al 2020 Nat Rev Microbiol 18:67-83)) with their corresponding guide RNA(s), TALENs, zinc finger nucleases (ZFNs), meganucleases, and the like. Alternatively, a deactivated CRISPR nuclease (e.g., a deactivated Cas9, Cas12a, or Cms1 endonuclease) fused to a transcriptional regulatory element can be targeted to the upstream regulatory region of a polynucleotide of interest, thereby modulating the function of the polynucleotide of interest (Piatek et al. (2015) Plant Biotechnol J 13:578-589).

Any editing reagents for use in any genome-editing methods including those described herein can be operably linked to the promoter of the present disclosure and expressed in a plant or plant part.

Methods disclosed herein include conferring desired traits to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain nonlimiting embodiments, the trait is conferred to the plant by introducing a nucleic acid sequence (e.g. using plant transformation methods) that encodes production of a certain protein by the plant. In certain nonlimiting embodiments, the desired trait is conferred to a plant by causing a null mutation in the plant's genome (e.g. when the desired trait is reduced expression or no expression of a certain trait).

In certain nonlimiting embodiments, the desired trait is conferred to a plant by crossing two plants to create offspring that express the desired trait. It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

In certain embodiments, a user can combine the teachings herein with high-density molecular marker profiles spanning substantially the entire genome of a plant to estimate the value of selecting certain candidates in a breeding program in a process commonly known as genome selection.

### D. Transformation of Plants

Provided herein are methods for transforming plants or plant parts by introducing into the plants or plant parts a construct for expressing a polynucleotide of interest or for introducing one or more mutations (e.g., insertions, substitutions, or deletions) at a desired target site in the plant genome, wherein the construct comprises a promoter disclosed herein. The term “transform” or “transformation” refers to any method used to introduce polypeptides or polynucleotides into plant cells. For purpose of the present disclosure, the transformation can be: “stable transformation”, wherein the transformation construct (e.g., a construct comprising sequences encoding guide RNA and/or a nuclease for use in the methods of the present invention) is introduced into a host (e.g., a host plant, plant part, plant cell, etc.) and integrates into the genome of the host and is capable of being inherited by the progeny thereof, or “transient transformation”, wherein the transformation construct (e.g., a construct comprising a guide RNA and/or a polynucleotide encoding a nuclease for use in the methods of the present invention) is introduced into a host (e.g., a host plant, plant part, plant cell, etc.) and expressed temporarily. The methods disclosed herein can also be used for insertion of heterologous polynucleotides and/or modification of native plant gene expression to achieve desirable plant traits, e.g., increased sugar content.

Sequences encoding any polynucleotide of interest operably linked to a promoter disclosed herein can be introduced into a plant cell, organelle, or plant embryo by a variety of means of transformation, including microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J 3:2717-2722), and ballistic particle acceleration [see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens)]; all of which are herein incorporated by reference.

Agrobacterium- and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, Agrobacterium and viral mediated (Caulimoriviruses, Geminiviruses, RNA plant viruses), liposome mediated and the like.

The nuclease polypeptides (or encoding nucleic acid), the guide RNA(s) (or DNAs encoding the guide RNA), and the optional donor polynucleotide(s) can be introduced into the plant cell, organelle, or plant embryo simultaneously or sequentially. The ratio of the nuclease (or encoding nucleic acid) to the guide RNA(s) (or encoding DNA) generally will be about stoichiometric such that the two components can form an RNA-protein complex with the target DNA. In one embodiment, DNA encoding a nuclease and DNA encoding a guide RNA are delivered together within the plasmid vector.

The cells that have been transformed may be cultured and grown into plants in accordance with conventional ways. See, for example, McCormick et al. (1986) Plant Cell Reports 5:81-84. In this manner, the present invention provides transformed plants or plant parts, transformed seed (also referred to as “transgenic seed”) or transformed plant progenies having a nucleic acid modification stably incorporated into their genome.

The present invention may be used for transformation of any plant species, e.g., both monocots and dicots. The present invention can be used for transformation of crop plants or part of crop plants, e.g., corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp., such as tepary bean (Phaseolus acutifolius), lima bean (Phaseolus lunatus), common bean (Phaseolus vulgaris)), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Viciafaba), mung bean (Vigna radiata), lupins (Lupinus spp., such as white lupin (Lupinus albus)), mesquite (Prosopis spp.), carob (Ceratonia siliqua), tamarind (Tamarindus indica), alfalfa (Medicago sativa), Lotus japonicus, and clover (Trifolium spp.). Additionally or alternatively, the present invention can be used for transformation of an oilseed plant (e.g., canola (Brassica napus), cotton (Gossypium spp.), camelina (Camelina sativa) and sunflower (Helianthus spp.)), or other species including wheat (Triticum spp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of Pisum sativum or a part of Pisum sativum.

Also provided herein are plants and plant parts generated by the methods of the present disclosure, and plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant concentrate (e.g., whole plant concentrate or plant part concentrate), plant powder [e.g., formulated powder, such as formulated plant part powder (e.g., seed flour)], and plant biomass (e.g., dried biomass, such as crushed and/or powdered biomass), and food or beverage products obtained from plants of the present disclosure. Also provided herein are seeds, such as a representative sample of seeds, from a plant generated by the methods of the present disclosure.

### VII. Breeding of Plants

Disclosed herein are methods for breeding a plant, such as a plant comprising a promoter molecule and/or a DNA construct of the present disclosure, or a plant generated according to the methods of the present disclosure. A plant containing the one or more heterogeneous nucleic acid sequences of the present disclosure may be regenerated from a plant cell or plant part, wherein the genome of the plant cell or plant part is genetically-modified to contain the one or more mutations or the polynucleotide of the present disclosure. Using conventional breeding techniques or self-pollination, one or more seeds may be produced from the plant that contains the one or more mutations or the polynucleotide of the present disclosure. Such a seed, and the resulting progeny plant grown from such a seed, may contain the one or more mutations or the polynucleotide of the present disclosure, and therefore may be transgenic. Progeny plants are plants having a genetic modification to contain the one or more mutations or the polynucleotide of the present disclosure, which descended from the original plant having modification to contain the one or more mutations or the polynucleotide of the present disclosure. Seeds produced using such a plant of the invention can be harvested and used to grow generations of plants having genetic modification to contain the one or more mutations or the polynucleotide of the present disclosure, e.g., progeny plants, of the invention, comprising the polynucleotide and optionally expressing a polynucleotide of agronomic interest (e.g., herbicide resistance gene). Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

### EXAMPLES

The following examples are offered by way of illustration and not by way of limitation. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

### Example 1: Effect of PsUBI3p Linked to Guide RNA in Transfected Pea Protoplasts

Pisum sativum UBIQUITIN3 promoter (PsUBI3p) was operably linked to a guide RNA construct, co-transfected with a nuclease construct into pea leaf protoplast cells, and evaluated for the editing frequencies. In brief, protoplasts were isolated from Pisum sativum cv. “Maxum” leaves, and were transfected with a construct encoding the PsUBI3p operably linked to the guide RNA as well as a construct encoding the nuclease using a standard polyethylene glycol process. The editing frequencies of the target gene site were determined after 24-hour incubation by a standard droplet digital PCR analysis.

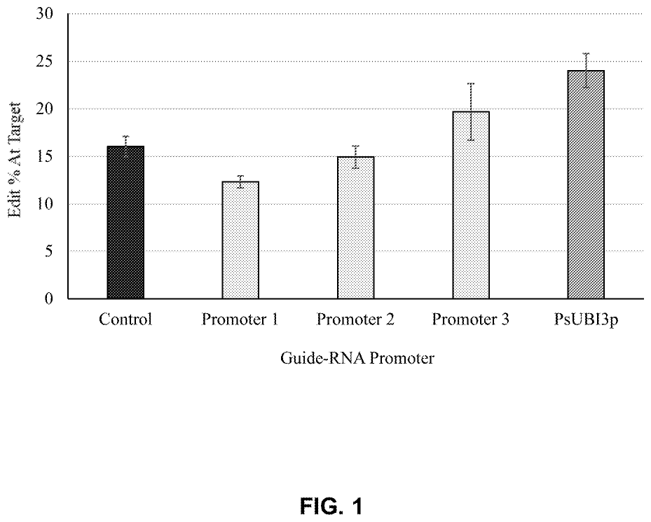

As shown in FIG. 1, PsUBI3p linked to the guide RNA showed a 1.5-fold higher editing frequency of a target compared to the control promoter (AtUBI11p) linked the guide RNA, demonstrating a statistically significant increase in editing efficiency (p=0.0077). The nucleic acid sequences of PsUBI3p and AtUBI11p are set forth as SEQ ID NOs: 6 and 43, respectively. Moreover, the PsUBI3p drove editing at a higher efficiency compared to other three guide RNA promoters tested (Promoters 1-3).

### Example 2: Effect of PsUBI3p Linked to Guide RNA and/or CRISPR-Cas12a Nuclease in Transfected Pea Protoplasts

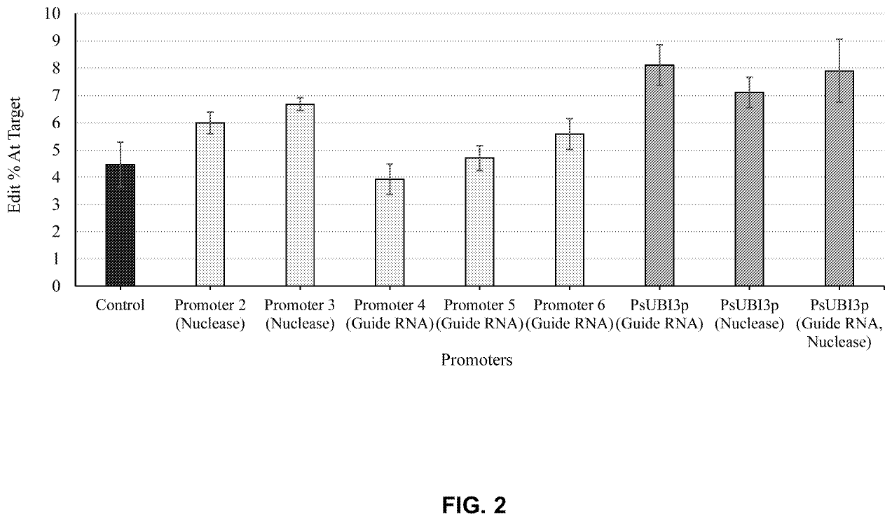

Promoters including PsUBI3p were fused to a CRISPR-Cas12a (Cpf1) nuclease construct and a guide RNA construct in operable linkage, transfected in pea leaf protoplast cells, and evaluated for editing frequencies at the target gene site as described in Example 1. Unless specified in FIG. 2, the guide RNA or the nuclease was fused to the control promoter.

As shown in FIG. 2, PsUBI3p produced editing of a target at a statistically significantly higher frequency compared to the control promoter (AtUBI11p) linked to the guide RNA and the nuclease:
1.8-fold higher editing frequency when PsUBI3p was linked to the guide RNA (p 0.0006);
1.6-fold higher editing frequency when PsUBI3p was linked to the nuclease (p 0.0025); and
1.8-fold higher editing frequency when PsUBI3p was linked to the guide RNA and the nuclease (p 0.004).
   Moreover, the editing frequency of PsUBI3p was higher compared to other two nuclease promoters (Promoters 2 and 3) and three guide RNA promoters (Promoters 4-6) tested.

### Example 3: Effect of PsUBI3p Linked to Guide RNA in Agrobacterium-Infiltrated Pea Leaves

To evaluate the effect of PsUBI3p on expression and function of a guide RNA in a longer term, the PsUBI3p-guide RNA construct was subcloned into a binary vector containing a AtUBI11p-nuclease cassette, and was delivered into Pisum sativum cv. “Maxum” leaves via Agrobacterium tumefaciens. The editing frequencies at the target gene site was evaluated after a 1-week incubation using the methods described in protoplast experiments.

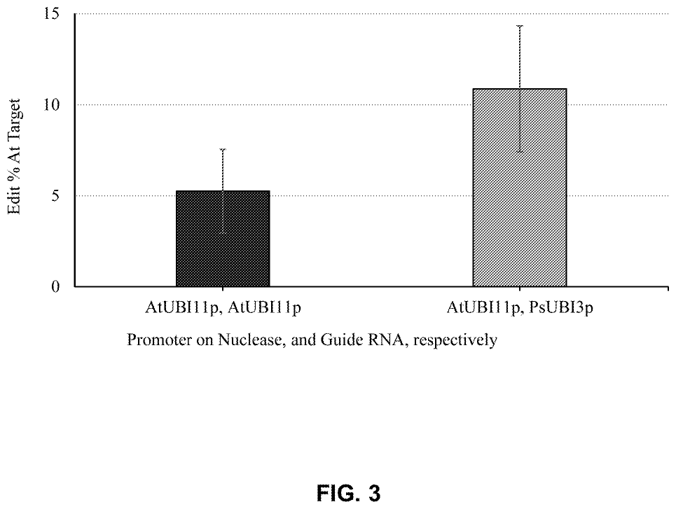

As shown in FIG. 3, PsUBI3p produced a statistically significant increase in editing frequencies compared to the control guide RNA promoter (AtUBI11p) (p 0.0019).

### Example 4: Effect of PsUBI3p Linked to Guide RNA and/or CRISPR-Cas12a Nuclease in Stable Regenerated Pea T0 Plants

The performance of PsUBI3p for editing in stably transformed pea plants was compared to that of AtUBI11p (control) at the target gene site. In brief, pea embryonic meristems were stably transformed with binary vectors capable of expressing the Mc.2Cpf1 nuclease operably linked to the promoter PsUBI3p or AtUBI11p (control) as well as the guide RNA operably linked to the promoter PsUBI3p or AtUBI11p (control).

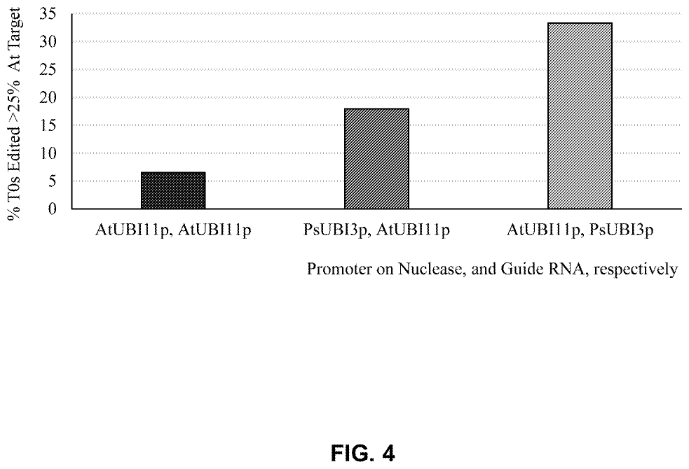

As shown in FIG. 4, editing frequencies were statistically significantly higher in plants in which the Mc.2 nuclease was expressed with AtUBI11p and the guide RNA was expressed with PsUBI3p as compared to control plants in which both the Mc.2 nuclease and guide RNA were linked to AtUBI11p (Fisher's exact test, p 0.008). Moreover, editing frequencies were higher in plants in which the Mc.2 nuclease was expressed with PsUBI3p and the guide RNA was expressed with AtUBI11p as compared to control plants

### Example 5: Generation of Promoters Comprising One or More Synthetic Motifs

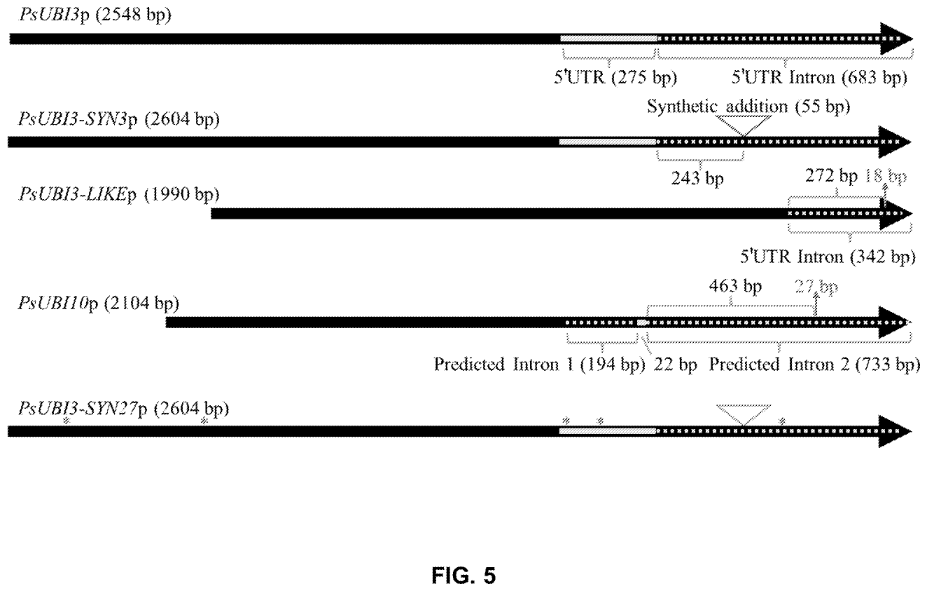

Diagrams of promoters PsUBI3p, PsUBI3-LIKEp, PsUBI10p, PsUBI3-SYN3p, and PsUBI3-SYN27p are depicted in FIG. 5. The top row depicts the PsUBI3p promoter, with the 5′UTR and 5′UTR intron shown in gray and dotted lines, respectively. The second, third, and fourth rows depict Pisum sativum PsUBI3-SYN3p, PsUBI3-LIKEp, and PsUBI10p respectively. The fifth row depicts PsUBI3-SYN27p. The nucleic acid sequences for PsUBI3p, PsUBI3-SYN3p, PsUBI3-LIKEp, PsUBI10p, and PsUBI3-SYN27p are set forth as SEQ ID NOs: 6, 11, 7, 8, and 79. The nucleic acid sequence for the PsUBI3p 5′UTR intron is set forth as SEQ ID NO: 4.

Synthetic promoters incorporating one or more copies of one or more synthetic motif sequences derived from PsUBI3-LIKEp and/or PsUBI10p are generated. The second row depicts an exemplary synthetic PsUBI3p-derivative PsUBI3-SYN3p, which includes synthetic motif sequences derived from PsUBI3-LIKEp and PsUBI10p. The PsUBI3-SYN3p synthetic insert sequence (SEQ ID NO: 3) includes an 18 bp motif sequence (SEQ ID NO: 1) from PsUBI3-LIKEp with two 5 bp linkers on both ends (5′ end linker: ACGTA, 3′ end linker: TTATG) and a 27 bp motif sequence (SEQ ID NO: 2) from PsUBI10p, fused together in a 5′ to 3′ orientation. Said synthetic insert sequence (SEQ ID NO: 3) was inserted into PsUBI3p at mid-intron, and a 1 bp was added 3′ to the site of insertion to reconstitute the MfeI site. The locations of the motif sequences in PsUBI3-LIKEp, PsUBI10p, and PsUBI-SYN3p are identified in FIG. 5 with arrows. The nucleotide sequences of PsUBI3-SYN3p and its 5′UTR intron are set forth as SEQ ID NOs: 11 and 5, respectively. The fifth row depicts an exemplary synthetic PsUBI3(-SYN3)p-derivative PsUBI3-SYN27p. This construct includes five localized substitutions at positions marked with stars.

The sequences of two additional exemplary synthetic PsUBI3p-derived promoters PsUBI3-SYN1p and PsUBI3-SYN2p are set forth as SEQ ID NOs: 9 and 10, respectively. In PsUBI3-SYN1p, the 18 bp motif sequence (SEQ ID NO: 1) from PsUBI3-LIKEp with two 5 bp linkers on both ends (5′ end linker: ACGTA, 3′ end linker: TTATG) was inserted into PsUBI3p. In PsUBI3-SYN2p, the 27 bp motif sequence (SEQ ID NO: 2) from PsUBI10p was inserted into PsUBI3p and a 1 bp was included 3′ to the site of insertion to reconstitute the MfeI site.

### Example 6: Effect of Synthetic PsUBI3p-Derivatives Linked to Guide RNA in Agrobacterium-infiltrated Pea Leaves

PsUBI3-SYN1p, PsUBI3-SYN2p, and PsUBI3-SYN3p were linked to a guide RNA, delivered into pea leaves via Agrobacterium tumefaciens, and were evaluated for their effect on editing efficiency. In brief, T-DNA constructs were prepared with a pea target guide RNA cassette in which the guide RNA is operably linked to PsUBI3-SYN1p, PsUBI3-SYN2p, or PsUBI3-SYN3p, as well as an AtUBI11p-Mc.2Cpf1 2C-NLS nuclease cassette. The constructs were delivered into pea leaves via Agrobacterium, and editing frequencies at the target gene site were evaluated as described above.

PsUBI3-SYN1p-guide RNA, which comprises a synthetic motif derived from PsUBI3-LIKEp, showed 0.9-fold (p=0.89) and 1.4-fold (p=0.25) changes in editing frequencies in pea leaves as compared to PsUBI3p-guide RNA in two separate experiments.

PsUBI3-SYN2p-guide RNA, which comprises a synthetic motif derived from PsUBI10p, showed 1.4-fold (p=0.26) and 2.2-fold (p=0.007) increases in editing frequencies in pea leaves as compared to PsUBI3p-guide RNA in two separate experiments.

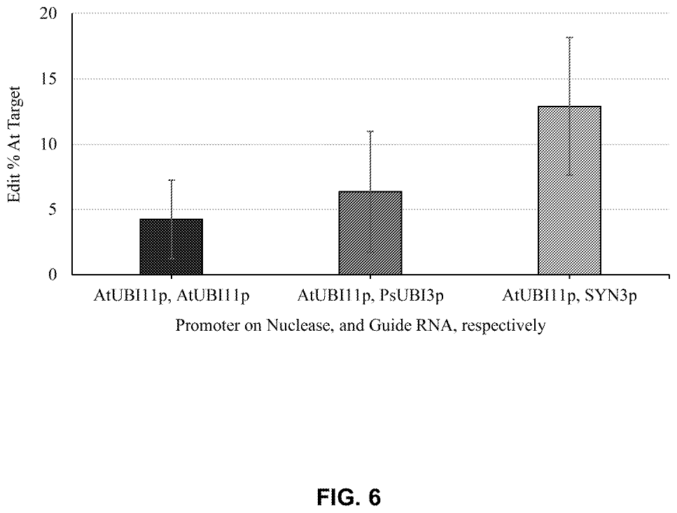

As shown in FIG. 6, PsUBI3-SYN3p-guide RNA, which comprises the synthetic motifs of both PsUBI3-SYN1p and PsUBI3-SYN2p, demonstrated a significant increase in editing frequencies in pea leaves as compared to PsUBI3p-guide RNA or AtUBI11p-guide RNA, showing 2-fold (p=0.02, FIG. 6) and 1.8-fold (p=0.009) increases in editing frequencies relative to PsUBI3p-guide RNA in two separate experiments.

### Example 7: Effect of PsUBI3p and PsUBI3-SYN3p Linked to Guide RNA and/or CRISPR-Cas12a Nuclease in Stable Regenerated Soybean T0 Plants

Soybean (Glycine max) embryonic meristems were stably transformed with constructs comprising promoters (PsUBI3p, PsUBI3-SYN3p, GmScreamM4p, 35S-ENp, and/or AtUBI11p) individually fused to a CRISPR-Cas12a (Cpf1) nuclease and promoters (PsUBI3p, PsUBI3-SYN3p, AtU6-26p, and/or AtUBI11p) individually operably linked to a guide RNA via Agrobacterium tumefaciens and editing frequencies at the target gene site was evaluated in T0 plants. The nucleic acid sequences for GmScreamM4p, 35S-ENp, and AtU6-26p are set forth as SEQ ID NOs: 41, 42, and 44, respectively.

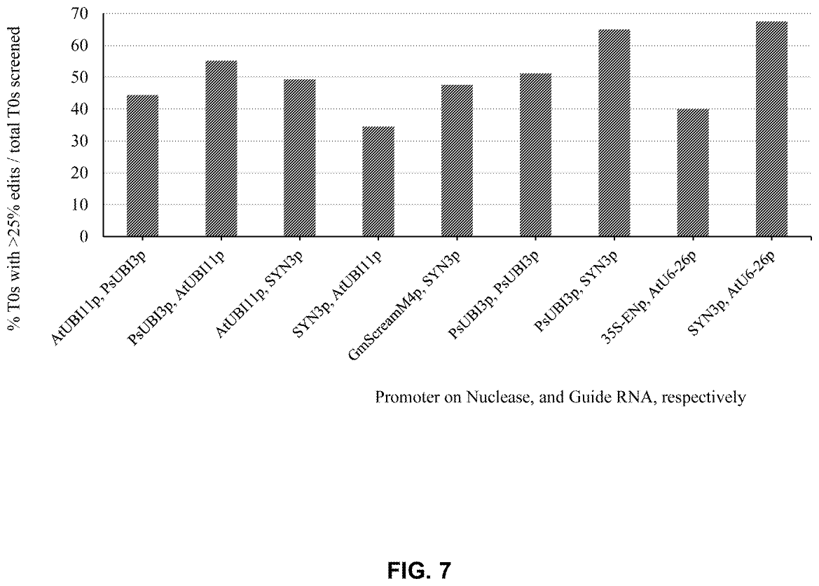

As shown in FIG. 7, PsUBI3p and PsUBI3-SYN3p were associated with higher percentages of T0 plants having >25% edits compared to other promoters. In particular, PsUBI3-SYN3p resulted in more percentages of plants with edits >25% over the total number screened (p 0.021) compared to the 35S promoter (35S-ENp) for driving the nuclease expression, when used in combination with the AtU6-26p for driving guide RNA expression.

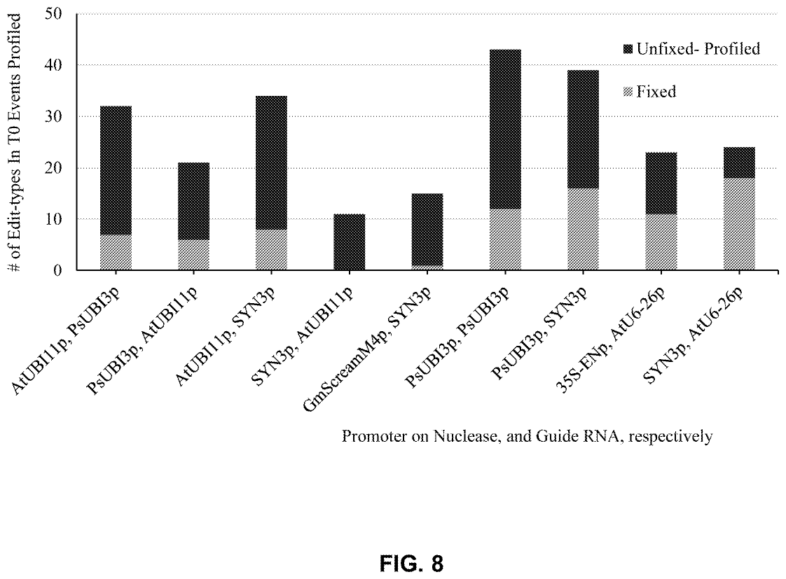

Further, the incidence of overall editing events in T0 plants, which comprise the incidence of “fixed” edit events (i.e., a consistent insertion-deletion profile across proliferating tissues in a mid-development T0 plant) and the incidence of “unfixed” or mosaic edits (i.e., an inconsistent insertion-deletion profile within the T0 plant tissues) were analyzed across soybean plants stably transformed with guide RNA and a CRISPR-Cas12a nuclease each operably linked to various promoters as described above. As shown in FIG. 8, fixed edits were more likely to be heritable than mosaic edits, due to their occurrence inferably earlier than germline-establishment and seed-set. In the plants comprising PsUBI3-SYN3p-nuclease and AtU6-26p-guide RNA constructs, the majority of edit events were fixed edits.

### Example 8: Effect of PsUBI3-SYN3p Synthetic Motifs in a Solanum lycopersicon Promoter

Synthetic motifs used to develop PsUBI3-SYN3p from PsUBI3p were tested for the ability to enhance editing frequencies of another promoter. Motifs from PsUBI3-SYN3p were introduced into the Solanum Lycopersicon UBIQUITIN 7 promoter (SlUBI7p) to generate SlUBI7-SYN1p. SlUBI7p and SlUBI7-SYN1p were individually, operably linked to a CRISPR-Cas12a (Cpf1) nuclease construct and co-transfected with a guide RNA construct operably linked to a control promoter (AtUBI11p) into soybean cotyledon protoplast cells. Incubation and evaluation of editing frequencies was conducted as described in Example 1.

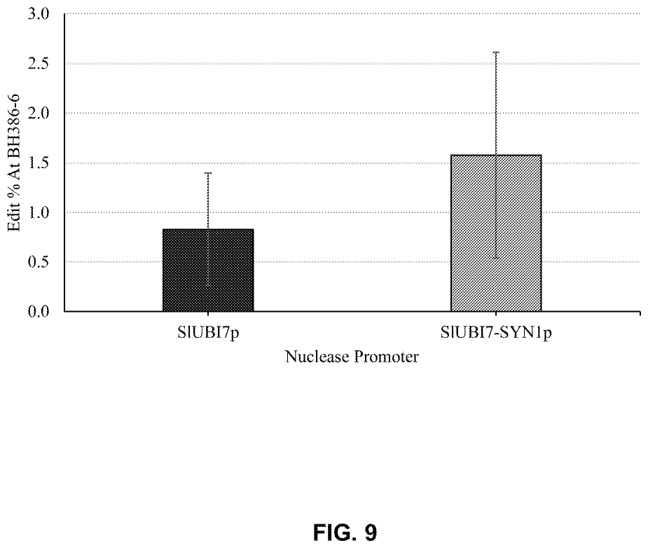

As shown in FIG. 9, SlUBI7-SYN1p linked to the nuclease showed a statistically significant (p=0.033), 1.9-fold higher editing frequency of a target compared to the control promoter (SlUBI7p) linked to the nuclease. The nucleic acid sequences for SlUBf7p and SlUBI7-SYN1p are set forth as SEQ ID NOs: 80 and 81, respectively.

Although the foregoing invention has been described in some detail by way of illustration and example for purposes of clarity of understanding, it will be obvious that certain changes and modifications may be practiced within the scope of the appended claims.

TABLE 2
Sequence Table

| SEQ ID | |
|---|---|
| NO: | DESCRIPTION |
| 1 | Nucleic acid sequence for motif from PsUBI3-LIKEp |
| 2 | Nucleic acid sequence for motif from PsUBI10p |
| 3 | Nucleic acid sequence for motif from PsUBI3-SYN3p |
| 4 | Nucleic acid sequence for PsUBI3p_5′UTR_Intron |
| 5 | Nucleic acid sequence for PsUBI3-SYN3p_5′UTR_Intron |
| 6 | Nucleic acid sequence for PsUBI3p |
| 7 | Nucleic acid sequence for PsUBI3-LIKEp |
| 8 | Nucleic acid sequence for PsUBI10p |
| 9 | Nucleic acid sequence for PsUBI3-SYN1p |
| 10 | Nucleic acid sequence for PsUBI3-SYN2p |
| 11 | Nucleic acid sequence for PsUBI3-SYN3p |
| 12 | Nucleic acid sequence for AhUBI3p |
| 13 | Nucleic acid sequence for AhUBI3-LIKEp |
| 14 | Nucleic acid sequence for AhUBI4p |
| 15 | Nucleic acid sequence for CaUBI3p |
| 16 | Nucleic acid sequence for CaUBI4p |
| 17 | Nucleic acid sequence for LaUBI3p |
| 18 | Nucleic acid sequence for LaUBI4Ap |
| 19 | Nucleic acid sequence for LaUBI4Bp |
| 20 | Nucleic acid sequence for LaUBBp |
| 21 | Nucleic acid sequence for LjUBI3p |
| 22 | Nucleic acid sequence for LjUBI3-LIKEp |
| 23 | Nucleic acid sequence for LjUBI4p |
| 23 | Nucleic acid sequence for PaUBI3-LIKEp |
| 25 | Nucleic acid sequence for PaUBI4p |
| 26 | Nucleic acid sequence for PaUBBp |
| 27 | Nucleic acid sequence for PlUBI3Ap |
| 28 | Nucleic acid sequence for PlUBI3Bp |
| 29 | Nucleic acid sequence for PlUBBp |
| 30 | Nucleic acid sequence for PvUBI3p |
| 31 | Nucleic acid sequence for PvUBI4p |
| 32 | Nucleic acid sequence for PvUBBp |
| 33 | Nucleic acid sequence for VuUBI3p |
| 34 | Nucleic acid sequence for VuUBI4p |
| 35 | Nucleic acid sequence for VuUBBp |
| 36 | Nucleic acid sequence for AtUBI10p |
| 37 | Nucleic acid sequence for GmUBIp |
| 38 | Nucleic acid sequence for GmYAO-1p |
| 39 | Nucleic acid sequence for AtRPS5Ap |
| 40 | Nucleic acid sequence for GmDMC1p |
| 41 | Nucleic acid sequence for GmScreamM4p |
| 42 | Nucleic acid sequence for 35S-ENp |
| 43 | Nucleic acid sequence for AtUBI11p |
| 44 | Nucleic acid sequence for AtU6-26p |
| 45 | Nucleic acid sequence for Mt7SLp |
| 46 | Nucleic acid sequence for Ps7SL2-1p |
| 47 | Nucleic acid sequence for Ps7SL4-1p |
| 48 | Nucleic acid sequence for Ps7SL4-2p |
| 49 | Nucleic acid sequence for Ps7SL6-1p |
| 50 | Nucleic acid sequence for Ps7SL7-1p |
| 51 | Nucleic acid sequence for Ps7SL7-2p |
| 52 | Nucleic acid sequence for Ps7SL7-3p |
| 53 | Nucleic acid sequence for Ps7SL7-4p |
| 54 | Nucleic acid sequence for PsU6-Ap |
| 55 | Nucleic acid sequence for PsU6-Bp |
| 56 | Nucleic acid sequence for PsU6-Cp |
| 57 | Nucleic acid sequence for PsU6-Dp |
| 58 | Nucleic acid sequence for PsU6-Ep |
| 59 | Nucleic acid sequence for MtU3-Bp |
| 60 | Nucleic acid sequence for PsU3B-1p |
| 61 | Nucleic acid sequence for PsU3B-2p |
| 62 | Nucleic acid sequence for PsU3B-3p |
| 63 | Nucleic acid sequence for PsU3B-4p |
| 64 | Nucleic acid sequence for PsU3B-5p |
| 65 | Nucleic acid sequence for PsU3B-6p |
| 66 | Nucleic acid sequence for PsU3B-7p |
| 67 | Nucleic acid sequence for PsU3B-8p |
| 68 | Nucleic acid sequence for PsU3B-9p |
| 69 | Nucleic acid sequence for PsU3B-10p |
| 70 | Nucleic acid sequence for PsU3B-11p |
| 71 | Nucleic acid sequence for PsU6-Fp |
| 72 | Nucleic acid sequence for PsU6-Hp |
| 73 | Nucleic acid sequence for MtU6-4p |
| 74 | Nucleic acid sequence for GmU6-Cp |
| 75 | Nucleic acid sequence for GmU6-Ip |
| 76 | Nucleic acid sequence for AtU3-Bp |
| 77 | Nucleic acid sequence for Gm7SLp |
| 78 | Amino acid sequence for McCpf1 |
| 79 | Nucleic acid sequence for PsUBI3-SYN27p |
| 80 | Nucleic acid sequence for SlUBI7p |
| 81 | Nucleic acid sequence for SlUBI7-SYN1p |

What is claimed is:

1. A nucleic acid molecule comprising a promoter sequence, wherein the promoter sequence has transcription initiation function and comprises one or more synthetic motif sequences, wherein each synthetic motif sequence comprises: (i) a nucleic acid sequence that has at least 95% sequence identity to any one of SEQ ID NOs: 1-3; or (ii) the nucleic acid sequence of any one of SEQ ID NOs: 1-3.

2. The nucleic acid molecule of claim 1 further comprising one or more linkers, wherein:
one of the one or more linkers connects the one or more synthetic motif sequences and the promoter sequence; and/or
the one or more synthetic motif sequences are at least two synthetic motif sequences, and wherein one of the one or more linkers connects two of the at least two synthetic motif sequences.

3. The nucleic acid molecule of claim 1 or 2, wherein the promoter sequence comprises two synthetic motif sequences, wherein each synthetic motif sequence comprises: (i) a nucleic acid sequence that has at least 95% sequence identity to any one of SEQ ID NOs: 1-3; or (ii) the nucleic acid sequence of any one of SEQ ID NOs: 1-3.

4. The nucleic acid molecule of claim 3, wherein said two synthetic motif sequences are different synthetic motif sequences.

5. The nucleic acid molecule of any one of claims 2-4, wherein at least one of said synthetic motif sequences comprise a nucleic acid sequence that has at least 95% sequence identity to SEQ ID NO: 1, or the nucleic acid sequence of SEQ ID NO: 1; and at least one of said synthetic motif sequences comprise a nucleic acid sequence that has at least 95% sequence identity to SEQ ID NO: 2, or the nucleic acid sequence of SEQ ID NO: 2.

6. The nucleic acid molecule of any one of claims 1-5, wherein said one or more synthetic motif sequences are inserted into the 5′ untranslated region (UTR) of said promoter sequence.

7. The nucleic acid molecule of any one of claims 1-6, wherein said one or more synthetic motif sequences are inserted into a 5′ intron of said promoter sequence.

8. The nucleic acid molecule of any one of claims 1-7, wherein the promoter sequence comprises: (i) a nucleic acid sequence that has at least 80% sequence identity to SEQ ID NO: 6, further comprising the one or more synthetic motif sequences inserted therein; or (ii) the nucleic acid sequence of SEQ ID NO: 6, further comprising the one or more synthetic motif sequences inserted therein.

9. The nucleic acid molecule of any one of claims 1-8, wherein the promoter sequence comprises: (i) a nucleic acid sequence that has at least 80% sequence identity to any one of SEQ ID NOs: 9-11 and 79-81, or (ii) the nucleic acid sequence of any one of SEQ ID NOs: 9-11 and 79-81.

10. The nucleic acid molecule of any one of claims 1-9, wherein the promoter sequence comprises at least one mutation cluster at positions selected the following positions:
a) at least one of 167, 169, 170, 171, 176, 177, 178, 179, and 180;
b) at least one of 561, 562, 563, 570, 571, 572, 573, 574;
c) at least one of 1446, 1607, 1608, 1609, 1611, 1617, 1618, 1619;
d) at least one of 1706, 1707, 1708, 1709, 1716, 1718, 1719, 1894; and
e) at least one of 2229, 2230, 2231, 2232, 2237, 2238, 2239, 2240, 2241.

11. The nucleic acid molecule of claim 10, wherein the promoter sequence comprises at least one mutation cluster selected from the following mutation clusters:
a) at least one of C167G, A169G, T170A, T171C, T176G, C177G, T178C, T179A, T180G;
b) at least one of T561G, A562T, T563G, T570G, T571G, A572C, T573A, T574G;
c) at least one of C1446G, T1607G, C1608T, A1609G, G1611C, T1617G, T1618C, T1619A;
d) at least one of T1706G, G1707T, T1708G, C1709A, T1716G, G1718A, C1719G, C1894A; and
e) at least one of A2229T, A2230G, G2231A, G2232C, C2237G, C2238G, A2239C, T2240A, T2241G.

12. A DNA construct comprising, in operable linkage:
(a) the nucleic acid molecule of any one of claims 1-9; a promoter molecule comprising a nucleic acid sequence that has at least 80% sequence identity to any one of SEQ ID NOs: 6-8 and 12-35, and retains transcription initiation function; or a promoter molecule comprising a nucleic acid sequence of any one of SEQ ID NOs: 6-8 and 12-35; and
(b) a polynucleotide of interest.

13. The DNA construct of claim 12, wherein the polynucleotide of interest encodes a guide RNA (guide RNA) and/or a nuclease.

14. The DNA construct of claim 12 or 13, wherein the polynucleotide of interest encodes a guide RNA, and wherein the DNA construct further comprises, in operable linkage:
(a) the nucleic acid molecule of any one of claims 1-9; a promoter molecule comprising a nucleic acid sequence that has at least 80% sequence identity to any one of SEQ ID NOs: 6-43 and 79-81, and retains transcription initiation function; or a promoter molecule comprising a nucleic acid sequence of any one of SEQ ID NOs: 6-43 and 79-81; and
(b) a polynucleotide of interest encoding a nuclease.

15. The DNA construct of claim 12 or 13, wherein the polynucleotide of interest encodes a nuclease, and wherein the DNA construct further comprises, in operable linkage:
(a) the nucleic acid molecule of any one of claims 1-9; a promoter molecule comprising a nucleic acid sequence that has at least 80% sequence identity to any one of SEQ ID NOs: 6-35, 43-77 and 79-81, and retains transcription initiation function; or a promoter molecule comprising a nucleic acid sequence of any one of SEQ ID NOs: 6-35, 43-77 and 79-81; and
(b) a polynucleotide of interest encoding a guide RNA.

16. The DNA construct of any one of claims 12-15, wherein the nuclease is a clustered, regularly interspaced, short palindromic repeats (CRISPR)-associated Cas endonuclease.

17. The DNA construct of claim 16, wherein the CRISPR nuclease is a Cas12a nuclease.

18. The DNA construct of claim 17, wherein the Cas12a nuclease is a McCpf1.

19. The DNA construct of any one of claims 13-18, wherein the nuclease is further operably linked to one or more nuclear localization sequences (NLSs) and/or one or more epitope tags.

20. The DNA construct of any one of claims 13-19, further comprising, in operable linkage, a nucleic acid molecule encoding a selectable marker and/or a regulatory RNA.

21. The DNA construct of claim 20, further comprising a promoter molecule operably linked to the regulatory RNA and comprising: (i) a nucleic acid sequence that has at least 80% identity to any one of SEQ ID NOs: 6-35, 43-77, and 79-81, and retains transcription initiation function; or (ii) a nucleic acid sequence of any one of SEQ ID NOs: 6-35, 43-77, and 79-81.

22. A cell comprising the nucleic acid molecule of any one of claims 1-11 or the DNA construct of any one of claims 12-121.

23. The cell of claim 120, wherein the cell is selected from the group consisting of a plant cell, a bacterial cell, and a fungal cell.

24. A plant or plant part comprising the nucleic acid molecule of any one of claims 1-11 or the DNA construct of any one of claims 12-21.

25. A plant or plant part comprising, in operable linkage:
(a) a promoter molecule having transcription initiation function and comprising one or more synthetic motif sequences, wherein each synthetic motif sequence comprises: (i) a nucleic acid sequence that has at least 95% sequence identity to any one of SEQ ID NOs: 1-3; or (ii) the nucleic acid sequence of any one of SEQ ID NOs: 1-3; and
(b) a polynucleotide of interest.

26. The plant or plant part of claim 25, wherein the promoter molecule further comprises one or more linkers, wherein:
one of the one or more linkers connects the one or more synthetic motif sequences and a promoter sequence; and/or
the one or more synthetic motif sequences are at least two synthetic motif sequences, and wherein one of the one or more linkers connects two of the at least two synthetic motif sequences.

27. The plant or plant part of claim 25 or 26, wherein the promoter molecule comprises two synthetic motif sequences, wherein each synthetic motif sequence comprises: (i) a nucleic acid sequence that has at least 95% sequence identity to any one of SEQ ID NOs: 1-3; or (ii) the nucleic acid sequence of any one of SEQ ID NOs: 1-3.

28. The plant or plant part of claim 27, wherein said two synthetic motif sequences are different synthetic motif sequences.

29. The plant or plant part of any one of claims 26-28, wherein at least one of said synthetic motif sequences comprise a nucleic acid sequence that has at least 95% sequence identity to SEQ ID NO: 1, or the nucleic acid sequence of SEQ ID NO: 1; and at least one of said synthetic motif sequences comprise a nucleic acid sequence that has at least 95% sequence identity to SEQ ID NO: 2, or the nucleic acid sequence of SEQ ID NO: 2.

30. The plant or plant part of any one of claims 25-29, wherein said one or more synthetic motif sequences are inserted into the 5′ UTR of said promoter molecule.

31. The plant or plant part of any one of claims 25-30, wherein said one or more synthetic motif sequences are inserted into a 5′ intron of said promoter molecule.

32. The plant or plant part of claim any one of claims 25-31, wherein the promoter molecule comprises: (i) a nucleic acid sequence that has at least 80% sequence identity to SEQ ID NO: 6, further comprising the one or more motif sequences inserted therein; or (ii) a nucleic acid sequence of SEQ ID NO: 6, further comprising the one or more motif sequences inserted therein.

33. The plant or plant part of claim any one of claims 25-32, wherein the promoter molecule comprises: (i) a nucleic acid sequence that has at least 80% sequence identity to any one of SEQ ID NOs: 9-11 and 79-81 or (ii) the nucleic acid sequence of any one of SEQ ID NOs: 9-11 and 79-81.

34. The plant or plant part of any one of claims 25-33, wherein the promoter sequence comprises at least one mutation cluster at positions selected the following position clusters:
f) at least one of 167, 169, 170, 171, 176, 177, 178, 179, and 180;
g) at least one of 561, 562, 563, 570, 571, 572, 573, 574;
h) at least one of 1446, 1607, 1608, 1609, 1611, 1617, 1618, 1619;
i) at least one of 1706, 1707, 1708, 1709, 1716, 1718, 1719, 1894; and
j) at least one of 2229, 2230, 2231, 2232, 2237, 2238, 2239, 2240, 2241.

35. The plant or plant part of claim 34, wherein the promoter sequence comprises at least one mutation cluster selected from the following mutation clusters:
f) at least one of C167G, A169G, T170A, T171C, T176G, C177G, T178C, T179A, T180G;
g) at least one of T561G, A562T, T563G, T570G, T571G, A572C, T573A, T574G;
h) at least one of C1446G, T1607G, C1608T, A1609G, G1611C, T1617G, T1618C, T1619A;
i) at least one of T1706G, G1707T, T1708G, C1709A, T1716G, G1718A, C1719G, C1894A; and
j) at least one of A2229T, A2230G, G2231A, G2232C, C2237G, C2238G, A2239C, T2240A, T2241G.

36. The plant or plant part of any one of claims 24-35, wherein the promoter molecule(s) and/or the polynucleotide(s) of interest are stably inserted in the genome of said plant or plant part.

37. The plant or plant part of any one of claims 24-36, wherein said plant is selected from the group consisting of corn (Zea mays), Brassica species, Brassica napus, Brassica rapa, Brassica juncea, alfalfa (Medicago sativa), pea (Pisum sativum), fava bean (Vicia faba), common bean (Phaseolus vulgaris), chickpea (Cicer arietinum), mung bean (Vigna radiata), white lupin (Lupinus albus), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

38. The plant or plant part of claim 35, wherein said plant is Pisum sativum.

39. A method of expressing a polynucleotide of interest in a plant or plant part comprising introducing a DNA construct into the plant or plant part, wherein the DNA construct comprises, in operable linkage:
(a) a nucleic acid molecule of any one of claims 1-11;
a promoter molecule comprising a nucleic acid sequence that has at least 80% sequence identity to any one of SEQ ID NOs: 6-8 and 12-35, and retains transcription initiation function; or
a promoter molecule comprising a nucleic acid sequence of any one of SEQ ID NOs: 6-8 and 12-35; and
(b) a polynucleotide of interest.

40. A method of expressing a polynucleotide of interest in a plant or plant part comprising:
(i) introducing a DNA construct into a plant cell, wherein the DNA construct comprises, in operable linkage:
(a) a promoter molecule having transcription initiation function and comprising one or more synthetic motif sequences, wherein the one or more synthetic motif sequences each comprise:
(i) a nucleic acid sequence that has at least 95% sequence identity to any one of SEQ ID NOs: 1-3;
or (ii) the nucleic acid sequence of any one of SEQ ID NOs: 1-3; and
(b) the polynucleotide of interest; and
(ii) regenerating a plant or plant part from said plant cell.

41. The method of claim 40, wherein the DNA construct further comprises one or more linkers, wherein:
one of the one or more linkers connects the one or more synthetic motif sequences and the promoter molecule; and/or
the one or more synthetic motif sequences are at least two synthetic motif sequences, and wherein one of the one or more linkers connects two of the at least two synthetic motif sequences.

42. The method of claim 40 or 341, wherein the promoter molecule comprises two synthetic motif sequences, wherein each synthetic motif sequence comprises: (i) a nucleic acid sequence that has at least 95% sequence identity to any one of SEQ ID NOs: 1-3; or (ii) the nucleic acid sequence of any one of SEQ ID NOs: 1-3.

43. The method of claim 42, wherein said two synthetic motif sequences are different synthetic motif sequences.

44. The method of any one of claims 41-43, wherein at least one of said synthetic motif sequences comprise a nucleic acid sequence that has at least 95% sequence identity to SEQ ID NO: 1, or the nucleic acid sequence of SEQ ID NO: 1; and at least one of said synthetic motif sequences comprise a nucleic acid sequence that has at least 95% sequence identity to SEQ ID NO: 2, or the nucleic acid sequence of SEQ ID NO: 2.

45. The method of any one of claims 40-44, wherein said one or more synthetic motif sequences are inserted into the 5′ UTR of said promoter molecule.

46. The method of any one of claims 40-45, wherein said one or more synthetic motif sequences are inserted into a 5′ intron of said promoter molecule.

47. The method of any one of claims 40-46, wherein the promoter molecule comprises: (i) a nucleic acid sequence that has at least 80% sequence identity to SEQ ID NO: 6, further comprising the one or more synthetic motif sequences inserted therein; or (ii) the nucleic acid sequence of SEQ ID NO: 6, further comprising the one or more synthetic motif sequences inserted therein.

48. The method of any one of claims 40-47, wherein the promoter molecule comprises: (i) a nucleic acid sequence that has at least 80% sequence identity to any one of SEQ ID NOs: 9-11 and 79-81 or (ii) the nucleic acid sequence of any one of SEQ ID NOs: 9-11 and 79-81.

49. The method of any one of claims 40-48, wherein the promoter sequence comprises at least one mutation cluster at positions selected the following position clusters:
k) at least one of 167, 169, 170, 171, 176, 177, 178, 179, and 180;
l) at least one of 561, 562, 563, 570, 571, 572, 573, 574;
m) at least one of 1446, 1607, 1608, 1609, 1611, 1617, 1618, 1619;
n) at least one of 1706, 1707, 1708, 1709, 1716, 1718, 1719, 1894; and
o) at least one of 2229, 2230, 2231, 2232, 2237, 2238, 2239, 2240, 2241.

50. The nucleic acid molecule of claim 49, wherein the promoter sequence comprises at least one mutation cluster selected from the following mutation clusters:
k) at least one of C167G, A169G, T170A, T171C, T176G, C177G, T178C, T179A, T180G;
l) at least one of T561G, A562T, T563G, T570G, T571G, A572C, T573A, T574G;
m) at least one of C1446G, T1607G, C1608T, A1609G, G1611C, T1617G, T1618C, T1619A;
n) at least one of T1706G, G1707T, T1708G, C1709A, T1716G, G1718A, C1719G, C1894A; and
o) at least one of A2229T, A2230G, G2231A, G2232C, C2237G, C2238G, A2239C, T2240A, T2241G.

51. The method of any one of claims 39-50, wherein the polynucleotide of interest encodes a guide RNA (guide RNA) and/or a nuclease.

52. The method of any one of claims 39-51, wherein the polynucleotide of interest encodes a guide RNA, and wherein the DNA construct further comprises, in operable linkage:
(a) the nucleic acid molecule of any one of claims 1-9; a promoter molecule comprising a nucleic acid sequence that has at least 80% sequence identity to any one of SEQ ID NOs: 6-43, and retains transcription initiation function; or a promoter molecule comprising a nucleic acid sequence of any one of SEQ ID NOs: 6-43 and 79-81; and
(b) a nucleic acid molecule encoding a nuclease.

53. The method of any one of claims 39-51, wherein the polynucleotide of interest encodes a nuclease, and wherein the DNA construct further comprises, in operable linkage:
(a) the nucleic acid molecule of any one of claim 111; a promoter molecule comprising a nucleic acid sequence that has at least 80% sequence identity to any one of SEQ ID NOs: 6-35, 43-77 and 79-81, and retains transcription initiation function; or a promoter molecule comprising a nucleic acid sequence of any one of SEQ ID NOs: 6-35, 43-77 and 79-81; and
(b) a nucleic acid molecule encoding a guide RNA.

54. The method of any one of claims 51-53, wherein the nuclease is a clustered, regularly interspaced, short palindromic repeats (CRISPR)-associated Cas endonuclease.

55. The method of claim 54, wherein the CRISPR nuclease is a Cas12a nuclease.

56. The method of claim 55, wherein the Cas12a nuclease is McCpf1.

57. The method of any one of claims 51-56, wherein the nuclease is further operably linked to one or more nuclear localization sequences (NLSs) and/or one or more epitope tags.

58. The method of any one of claims 39-57, wherein the polynucleotide of interest is stably inserted into the genome of the plant or plant part.

59. The method of any one of claims 39-58, wherein expression or function of one or more molecules encoded by the polynucleotide of interest is increased in the plant or plant part relative to a control plant or plant part,
wherein in the control plant or plant part comprises the polynucleotide of interest operably linked to a control promoter that does not comprise the promoter molecule of any one of claims 35-44.

60. The method of claim 59, wherein the one or more molecules are a guide RNA and/or a nuclease, and wherein an efficiency of introducing a mutation to a genome of a plant or plant part is increased by about 10% to about 500% in the plant or plant part relative to the control plant or plant part.

61. The method of any one of claims 39-60, wherein the DNA construct further comprises, in operable linkage, a nucleic acid molecule encoding a selectable marker and/or a regulatory RNA.

62. The method of claim 61, wherein the DNA construct further comprises a promoter molecule operably linked to the regulatory RNA and comprising: (i) a nucleic acid sequence that has at least 80% identity to any one of SEQ ID NOs: 6-35, 43-77, and 79-81, and retains transcription initiation function; or (ii) a nucleic acid sequence of any one of SEQ ID NOs: 6-35, 43-77, and 79-81.

63. A plant or plant part produced by the method of any one of claims 39-62, wherein said plant or plant part comprises said DNA construct.
